SEQUENCE LISTING (391 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 117 aa, Artificial Sequence; identical to SEQ ID NO: 1
  Description of Artificial Sequence Synthetic polypeptide

SEQ ID NO: 4 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 5 - protein, 117 aa, Artificial Sequence; identical to SEQ ID NO: 1
  Description of Artificial Sequence Synthetic polypeptide

SEQ ID NO: 6 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 7 - protein, 117 aa, Artificial Sequence; identical to SEQ ID NO: 1
  Description of Artificial Sequence Synthetic polypeptide

SEQ ID NO: 8 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 117 aa, Artificial Sequence; identical to SEQ ID NO: 1
  Description of Artificial Sequence Synthetic polypeptide

SEQ ID NO: 10 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 11 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 12 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 13 - protein, 117 aa, Artificial Sequence; identical to SEQ ID NO: 1
  Description of Artificial Sequence Synthetic polypeptide

SEQ ID NO: 14 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 15 - protein, 117 aa, Artificial Sequence; identical to SEQ ID NO: 1
  Description of Artificial Sequence Synthetic polypeptide

SEQ ID NO: 16 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 17 - protein, 117 aa, Artificial Sequence; identical to SEQ ID NO: 1
  Description of Artificial Sequence Synthetic polypeptide

SEQ ID NO: 18 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 19 - protein, 117 aa, Artificial Sequence; identical to SEQ ID NO: 1
  Description of Artificial Sequence Synthetic polypeptide

SEQ ID NO: 20 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 21 - protein, 117 aa, Artificial Sequence; identical to SEQ ID NO: 1
  Description of Artificial Sequence Synthetic polypeptide

SEQ ID NO: 22 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 23 - protein, 117 aa, Artificial Sequence; identical to SEQ ID NO: 1
  Description of Artificial Sequence Synthetic polypeptide

SEQ ID NO: 24 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 25 - protein, 117 aa, Artificial Sequence; identical to SEQ ID NO: 1
  Description of Artificial Sequence Synthetic polypeptide

SEQ ID NO: 26 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 27 - protein, 117 aa, Artificial Sequence; identical to SEQ ID NO: 1
  Description of Artificial Sequence Synthetic polypeptide

SEQ ID NO: 28 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 29 - protein, 117 aa, Artificial Sequence; identical to SEQ ID NO: 1
  Description of Artificial Sequence Synthetic polypeptide

SEQ ID NO: 30 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 31 - protein, 117 aa, Artificial Sequence; identical to SEQ ID NO: 1
  Description of Artificial Sequence Synthetic polypeptide

SEQ ID NO: 32 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 33 - protein, 117 aa, Artificial Sequence; identical to SEQ ID NO: 1
  Description of Artificial Sequence Synthetic polypeptide

SEQ ID NO: 34 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 35 - protein, 117 aa, Artificial Sequence; identical to SEQ ID NO: 1
  Description of Artificial Sequence Synthetic polypeptide

SEQ ID NO: 36 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 37 - protein, 117 aa, Artificial Sequence; identical to SEQ ID NO: 1
  Description of Artificial Sequence Synthetic polypeptide

SEQ ID NO: 38 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 39 - protein, 117 aa, Artificial Sequence; identical to SEQ ID NO: 1
  Description of Artificial Sequence Synthetic polypeptide

SEQ ID NO: 40 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 41 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 42 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 43 - protein, 9 aa, Artificial Sequence
  Description of Artificial Sequence Synthetic peptide
GTFSSYAIS

SEQ ID NO: 44 - protein, 17 aa, Artificial Sequence
  Description of Artificial Sequence Synthetic peptide
GIIPIFGTANYAQKFQG

SEQ ID NO: 45 - protein, 18 aa, Artificial Sequence
  Description of Artificial Sequence Synthetic peptide
ARGDSSIRHAYYYYGMDV

SEQ ID NO: 46 - protein, 17 aa, Artificial Sequence
  Description of Artificial Sequence Synthetic peptide
KSSQSVLYSSNNKNYLA

SEQ ID NO: 47 - protein, 7 aa, Artificial Sequence
  Description of Artificial Sequence Synthetic peptide
WASTRES

SEQ ID NO: 48 - protein, 9 aa, Artificial Sequence
  Description of Artificial Sequence Synthetic peptide
QQYYSTPIT

SEQ ID NO: 49 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 50 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 51 - protein, 11 aa, Artificial Sequence
  Description of Artificial Sequence Synthetic peptide
GSISSSSYYWG

SEQ ID NO: 52 - protein, 16 aa, Artificial Sequence
  Description of Artificial Sequence Synthetic peptide
SIYYSGSTYYNPSLKS

SEQ ID NO: 53 - protein, 13 aa, Artificial Sequence
  Description of Artificial Sequence Synthetic peptide
ARGSDRFHPYFDY

SEQ ID NO: 54 - protein, 11 aa, Artificial Sequence
  Description of Artificial Sequence Synthetic peptide
RASQSVSRYLA

SEQ ID NO: 55 - protein, 7 aa, Artificial Sequence
  Description of Artificial Sequence Synthetic peptide
DASNRAT

SEQ ID NO: 56 - protein, 9 aa, Artificial Sequence
  Description of Artificial Sequence Synthetic peptide
QQFDTWPPT

SEQ ID NO: 57 - protein, 117 aa, Artificial Sequence; identical to SEQ ID NO: 1
  Description of Artificial Sequence Synthetic polypeptide

SEQ ID NO: 58 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 59 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 60 - protein, 113 aa, Artificial Sequence [sequence omitted]

SEQ ID NO: 61 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 62 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 63 - protein, 9 aa, Artificial Sequence
  Description of Artificial Sequence Synthetic peptide
YTFTSYYMH

SEQ ID NO: 64 - protein, 17 aa, Artificial Sequence
  Description of Artificial Sequence Synthetic peptide
IINPSGGSTSYAQKFQG

SEQ ID NO: 65 - protein, 19 aa, Artificial Sequence
  Description of Artificial Sequence Synthetic peptide
ARGAPNYGDTTHDYYYMDV

SEQ ID NO: 66 - protein, 11 aa, Artificial Sequence
  Description of Artificial Sequence Synthetic peptide
RASQSVSSNLA

SEQ ID NO: 67 - protein, 7 aa, Artificial Sequence
  Description of Artificial Sequence Synthetic peptide
GASTRAT

SEQ ID NO: 68 - protein, 9 aa, Artificial Sequence
  Description of Artificial Sequence Synthetic peptide
QQYDDWPFT

SEQ ID NO: 69 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 70 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 71 - protein, 9 aa, Artificial Sequence
  Description of Artificial Sequence Synthetic peptide
YTFTGYYMH

SEQ ID NO: 72 - protein, 17 aa, Artificial Sequence
  Description of Artificial Sequence Synthetic peptide
WINPNSGGTNYAQKFQG

SEQ ID NO: 73 - protein, 17 aa, Artificial Sequence
  Description of Artificial Sequence Synthetic peptide
ARDTGEYYDTDDHGMDV

SEQ ID NO: 74 - protein, 11 aa, Artificial Sequence
  Description of Artificial Sequence Synthetic peptide
RASQSVSSNLA

SEQ ID NO: 75 - protein, 7 aa, Artificial Sequence
  Description of Artificial Sequence Synthetic peptide
GASTRAT

SEQ ID NO: 76 - protein, 9 aa, Artificial Sequence
  Description of Artificial Sequence Synthetic peptide
QQDDYWPPT

SEQ ID NO: 77 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 78 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 79 - protein, 9 aa, Artificial Sequence
  Description of Artificial Sequence Synthetic peptide
FTFSSYAMS

SEQ ID NO: 80 - protein, 17 aa, Artificial Sequence
  Description of Artificial Sequence Synthetic peptide
AISGSGGSTYYADSVKG

SEQ ID NO: 81 - protein, 14 aa, Artificial Sequence
  Description of Artificial Sequence Synthetic peptide
AKDGGYYDSGAGDY

SEQ ID NO: 82 - protein, 11 aa, Artificial Sequence
  Description of Artificial Sequence Synthetic peptide
RASQGIDSWLA

SEQ ID NO: 83 - protein, 7 aa, Artificial Sequence
  Description of Artificial Sequence Synthetic peptide
AASSLQS

SEQ ID NO: 84 - protein, 9 aa, Artificial Sequence
  Description of Artificial Sequence Synthetic peptide
QQGVSYPRT

SEQ ID NO: 85 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 86 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 87 - protein, 9 aa, Artificial Sequence
  Description of Artificial Sequence Synthetic peptide
FTFSSYSMN

SEQ ID NO: 88 - protein, 17 aa, Artificial Sequence
  Description of Artificial Sequence Synthetic peptide
SISSSSSYIYYADSVKG

SEQ ID NO: 89 - protein, 15 aa, Artificial Sequence
  Description of Artificial Sequence Synthetic peptide
ARGAPMGAAAGWFDP

SEQ ID NO: 90 - protein, 11 aa, Artificial Sequence
  Description of Artificial Sequence Synthetic peptide
RASQGISSWLA

SEQ ID NO: 91 - protein, 7 aa, Artificial Sequence
  Description of Artificial Sequence Synthetic peptide
AASSLQS

SEQ ID NO: 92 - protein, 9 aa, Artificial Sequence
  Description of Artificial Sequence Synthetic peptide
QQGVSFPRT

SEQ ID NO: 93 - protein, 125 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 94 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 95 - protein, 9 aa, Artificial Sequence
  Description of Artificial Sequence Synthetic peptide
YTFTSYYMH

SEQ ID NO: 96 - protein, 17 aa, Artificial Sequence
  Description of Artificial Sequence Synthetic peptide
IINPSGGSTSYAQKFQG

SEQ ID NO: 97 - protein, 18 aa, Artificial Sequence
  Description of Artificial Sequence Synthetic peptide
AREGAGFAYGMDYYYMDV

SEQ ID NO: 98 - protein, 11 aa, Artificial Sequence
  Description of Artificial Sequence Synthetic peptide
RASQSVSSYLA

SEQ ID NO: 99 - protein, 7 aa, Artificial Sequence
  Description of Artificial Sequence Synthetic peptide
DASNRAT

SEQ ID NO: 100 - protein, 9 aa, Artificial Sequence
  Description of Artificial Sequence Synthetic peptide
QQSDNWPFT

SEQ ID NO: 101 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 102 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 103 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 104 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 105 - protein, 9 aa, Artificial Sequence
  Description of Artificial Sequence Synthetic peptide
GSFSGYYWS

SEQ ID NO: 106 - protein, 16 aa, Artificial Sequence
  Description of Artificial Sequence Synthetic peptide
EIDHSGSTNYNPSLKS

SEQ ID NO: 107 - protein, 11 aa, Artificial Sequence
  Description of Artificial Sequence Synthetic peptide
ARARGPWSFDP

SEQ ID NO: 108 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 109 - protein, 123 aa, Artificial Sequence
  Description of Artificial Sequence Synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSA

SEQ ID NO: 110 - protein, 7 aa, Artificial Sequence
  Description of Artificial Sequence Synthetic peptide
GFTFSSF

SEQ ID NO: 111 - protein, 6 aa, Artificial Sequence
  Description of Artificial Sequence Synthetic peptide
SSDSSA

SEQ ID NO: 112 - protein, 13 aa, Artificial Sequence
  Description of Artificial Sequence Synthetic peptide
GRENIYYGSRLDY

SEQ ID NO: 113 - protein, 108 aa, Artificial Sequence
  Description of Artificial Sequence Synthetic polypeptide
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKR

SEQ ID NO: 114 - protein, 8 aa, Artificial Sequence
  Description of Artificial Sequence Synthetic peptide
QNVDTNVA

SEQ ID NO: 115 - protein, 7 aa, Artificial Sequence
  Description of Artificial Sequence Synthetic peptide
SASYRYS

SEQ ID NO: 116 - protein, 9 aa, Artificial Sequence
  Description of Artificial Sequence Synthetic peptide
QQYNNYPFT

SEQ ID NO: 117 - protein, 119 aa, Artificial Sequence
  Description of Artificial Sequence Synthetic polypeptide
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAA

SEQ ID NO: 118 - protein, 5 aa, Artificial Sequence
  Description of Artificial Sequence Synthetic peptide
NYDIN

SEQ ID NO: 119 - protein, 11 aa, Artificial Sequence
  Description of Artificial Sequence Synthetic peptide
WIFPGDGSTQY

SEQ ID NO: 120 - protein, 9 aa, Artificial Sequence
  Description of Artificial Sequence Synthetic peptide
QTTATWFAY

SEQ ID NO: 121 - protein, 108 aa, Artificial Sequence
  Description of Artificial Sequence Synthetic polypeptide
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKR

SEQ ID NO: 122 - protein, 11 aa, Artificial Sequence
  Description of Artificial Sequence Synthetic peptide
RASQSISDYLH

SEQ ID NO: 123 - protein, 7 aa, Artificial Sequence
  Description of Artificial Sequence Synthetic peptide
YASQSIS

SEQ ID NO: 124 - protein, 9 aa, Artificial Sequence
  Description of Artificial Sequence Synthetic peptide
QNGHSFPLT

SEQ ID NO: 125 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 126 - protein, 20 aa, Artificial Sequence
  Description of Artificial Sequence Synthetic peptide
GGGGSGGGGSGGGGSGGGGS

SEQ ID NO: 127 - protein, 9 aa, Artificial Sequence
  Description of Artificial Sequence Synthetic peptide
GTFSSYAIS

SEQ ID NO: 128 - protein, 17 aa, Artificial Sequence
  Description of Artificial Sequence Synthetic peptide
GIIPIFGTANYAQKFQG

SEQ ID NO: 129 - protein, 19 aa, Artificial Sequence
  Description of Artificial Sequence Synthetic peptide
ARRGRKASGSFYYYYGMDV

SEQ ID NO: 130 - protein, 17 aa, Artificial Sequence
  Description of Artificial Sequence Synthetic peptide
ESSQSLLNSGNQKNYLT

SEQ ID NO: 131 - protein, 7 aa, Artificial Sequence
  Description of Artificial Sequence Synthetic peptide
WASTRES

SEQ ID NO: 132 - protein, 9 aa, Artificial Sequence
  Description of Artificial Sequence Synthetic peptide
QNDYSYPYT

SEQ ID NO: 133 - protein, 115 aa, Artificial Sequence
  Description of Artificial Sequence Synthetic polypeptide
EVQLQQSGAELVRPGALVKLSCKASGFNIKDYYMQWVKQRPEQGLEWIGWIDPENGKTVFDPKFRGKASISADTSSNTAYLQLSSLTSEDTAVYYCARWNPLAFWGQGTLVTVSS

SEQ ID NO: 134 - protein, 9 aa, Artificial Sequence
  Description of Artificial Sequence Synthetic peptide
FNIKDYYMQ

SEQ ID NO: 135 - protein, 17 aa, Artificial Sequence
  Description of Artificial Sequence Synthetic peptide
WIDPENGKTVFDPKFRG

SEQ ID NO: 136 - protein, 6 aa, Artificial Sequence
  Description of Artificial Sequence Synthetic peptide
WNPLAF

SEQ ID NO: 137 - protein, 107 aa, Artificial Sequence
  Description of Artificial Sequence Synthetic polypeptide
DIVMTQSQKFMSTSVGDRVSVTCKASQNVGTNVAWYQQKPGHSPKALIYSTSYRYSGVPDRFTGSGSGTDFTLTIRNVQSEDLAEYFCQQYNTYPYTFGGGTKLEIK

SEQ ID NO: 138 - protein, 11 aa, Artificial Sequence
  Description of Artificial Sequence Synthetic peptide
KASQNVGTNVA

SEQ ID NO: 139 - protein, 7 aa, Artificial Sequence
  Description of Artificial Sequence Synthetic peptide
STSYRYS

SEQ ID NO: 140 - protein, 9 aa, Artificial Sequence
  Description of Artificial Sequence Synthetic peptide
QQYNTYPYT

SEQ ID NO: 141 - protein, 121 aa, Artificial Sequence
  Description of Artificial Sequence Synthetic polypeptide
EVQLVESGGGVVQPGRSLRLSCAASGFTFSRFGMHWVRQAPGKGLEWVAFISNDGSNKYYADSVKGRFTISRDNSKNTLYLQMNSLRPEDTAVYYCARGRAYGSGSLFDPWGQGTLVTVSS

SEQ ID NO: 142 - protein, 5 aa, Artificial Sequence
  Description of Artificial Sequence Synthetic peptide
RFGMH

SEQ ID NO: 143 - protein, 16 aa, Artificial Sequence
  Description of Artificial Sequence Synthetic peptide
FISNDGSNKYYADSVK

SEQ ID NO: 144 - protein, 12 aa, Artificial Sequence
  Description of Artificial Sequence Synthetic peptide
GRAYGSGSLFDP

SEQ ID NO: 145 - protein, 108 aa, Artificial Sequence
  Description of Artificial Sequence Synthetic polypeptide
DIQLTQSPSSLSASVGDRVTITCRASRTISIYVNWYRQRPGKAPESLIYAASNLHSGVPSRFSGSGSGTDFTLTISSLQPEDFATYYCQQSIGRGVVTFGQGTKLEIK

SEQ ID NO: 146 - protein, 11 aa, Artificial Sequence
  Description of Artificial Sequence Synthetic peptide
RASRTISIYVN

SEQ ID NO: 147 - protein, 7 aa, Artificial Sequence
  Description of Artificial Sequence Synthetic peptide
AASNLHS

SEQ ID NO: 148 - protein, 10 aa, Artificial Sequence
  Description of Artificial Sequence Synthetic peptide
QQSIGRGVVT

SEQ ID NO: 149 - protein, 1257 aa, Homo sapiens
MVVALRYVWPLLLCSPCLLIQIPEEYEGHHVMEPPVITEQSPRRLVVFPTDDISLKCEASGKPEVQFRWTRDGVHFKPKEELGVTVYQSPHSGSFTITGNNSNFAQRFQGIYRCFASNKLGTAMSHEIRLMAEGAPKWPKETVKPVEVEEGESVVLPCNPPPSAEPLRIYWMNSKILHIKQDERVTMGQNGNLYFANVLTSDNHSDYICHAHFPGTRTIIQKEPIDLRVKATNSMIDRKPRLLFPTNSSSHLVALQGQPLVLECIAEGFPTPTIKWLRPSGPMPADRVTYQNHNKTLQLLKVGEEDDGEYRCLAENSLGSARHAYYVTVEAAPYWLHKPQSHLYGPGETARLDCQVQGRPQPEVTWRINGIPVEELAKDQKYRIQRGALILSNVQPSDTMVTQCEARNRHGLLLANAYIYVVQLPAKILTADNQTYMAVQGSTAYLLCKAFGAPVPSVQWLDEDGTTVLQDERFFPYANGTLGIRDLQANDTGRYFCLAANDQNNVTIMANLKVKDATQITQGPRSTIEKKGSRVTFTCQASFDPSLQPSITWRGDGRDLQELGDSDKYFIEDGRLVIHSLDYSDQGNYSCVASTELDVVESRAQLLVVGSPGPVPRLVLSDLHLLTQSQVRVSWSPAEDHNAPIEKYDIEFEDKEMAPEKWYSLGKVPGNQTSTTLKLSPYVHYTFRVTAINKYGPGEPSPVSETVVTPEAAPEKNPVDVKGEGNETTNMVITWKPLRWMDWNAPQVQYRVQWRPQGTRGPWQEQIVSDPFLVVSNTSTFVPYEIKVQAVNSQGKGPEPQVTIGYSGEDYPQAIPELEGIEILNSSAVLVKWRPVDLAQVKGHLRGYNVTYWREGSQRKHSKRHIHKDHVVVPANTTSVILSGLRPYSSYHLEVQAFNGRGSGPASEFTFSTPEGVPGHPEALHLECQSNTSLLLRWQPPLSHNGVLTGYVLSYHPLDEGGKGQLSFNLRDPELRTHNLTDLSPHLRYRFQLQATTKEGPGEAIVREGGTMALSGISDFGNISATAGENYSVVSWVPKEGQCNFRFHILFKALGEEKGGASLSPQYVSYNQSSYTQWDLQPDTDYEIHLFKERMFRHQMAVKTNGTGRVRLPPAGFATEGWFIGFVSAIILLLLVLLILCFIKRSKGGKYSVKDKEDTQVDSEARPMKDETFGEYRSLESDNEEKAFGSSQPSLNGDIKPLGSDDSLADYGGSVDVQFNEDGSFIGQYSGKKEKEAAGGNDSSGATSPINPAVALE

SEQ ID NO: 150 - protein, 127 aa, Artificial Sequence
  Description of Artificial Sequence Synthetic polypeptide
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSA

SEQ ID NO: 151 - protein, 7 aa, Artificial Sequence
  Description of Artificial Sequence Synthetic peptide
GFAFSSY

SEQ ID NO: 152 - protein, 6 aa, Artificial Sequence
  Description of Artificial Sequence Synthetic peptide
WYDGSN

SEQ ID NO: 153 - protein, 17 aa, Artificial Sequence
  Description of Artificial Sequence Synthetic peptide
DHYGSGVHHYFYYGLDV

SEQ ID NO: 154 - protein, 109 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKR

SEQ ID NO: 155 - protein, 9 aa, Artificial Sequence
  Description of Artificial Sequence Synthetic peptide
QSVSSSYLA

SEQ ID NO: 156 - protein, 7 aa, Artificial Sequence
  Description of Artificial Sequence Synthetic peptide
GASSRAT

SEQ ID NO: 157 - protein, 9 aa, Artificial Sequence
  Description of Artificial Sequence Synthetic peptide
QQYGSSPLT

SEQ ID NO: 158 - protein, 118 aa, Artificial Sequence
  Description of Artificial Sequence Synthetic polypeptide
QVQLQQSGAELVGPGSSVKISCKASGYAFSSYWMNWVKQRPGQGLEWIGQIYPGDGDTNYNGKFRGKVTLTADRSSSTADMQLSSLTSEDSAVYFCARDDGYEGFDYWGQGTTLTVSS

SEQ ID NO: 159 - protein, 7 aa, Artificial Sequence
  Description of Artificial Sequence Synthetic peptide
GYAFSSY

SEQ ID NO: 160 - protein, 6 aa, Artificial Sequence
  Description of Artificial Sequence Synthetic peptide
YPGDGD

SEQ ID NO: 161 - protein, 9 aa, Artificial Sequence
  Description of Artificial Sequence Synthetic peptide
DDGYEGFDY

SEQ ID NO: 162 - protein, 108 aa, Artificial Sequence
  Description of Artificial Sequence Synthetic polypeptide
DIVMTQSQKFMSTTVGDRVSLTCKASQSVGTAVAWYQEKTGQSPKLLIYSASNRYTGVPDRFTGSGSGTDFILTIRNMQSVDLADYFCQQYFTYPYTFGGGTKLEIQR

SEQ ID NO: 163 - protein, 8 aa, Artificial Sequence
  Description of Artificial Sequence Synthetic peptide
QSVGTAVA

SEQ ID NO: 164 - protein, 7 aa, Artificial Sequence
  Description of Artificial Sequence Synthetic peptide
SASNRYT

SEQ ID NO: 165 - protein, 9 aa, Artificial Sequence
  Description of Artificial Sequence Synthetic peptide
QQYFTYPYT

SEQ ID NO: 166 - protein, 117 aa, Artificial Sequence
  Description of Artificial Sequence Synthetic polypeptide
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSA

SEQ ID NO: 167 - protein, 7 aa, Artificial Sequence
  Description of Artificial Sequence Synthetic peptide
GFTFSSY

SEQ ID NO: 168 - protein, 6 aa, Artificial Sequence
  Description of Artificial Sequence Synthetic peptide
SSSSSY

SEQ ID NO: 169 - protein, 7 aa, Artificial Sequence
  Description of Artificial Sequence Synthetic peptide
VTDAFDI

SEQ ID NO: 170 - protein, 108 aa, Artificial Sequence
  Description of Artificial Sequence Synthetic polypeptide
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKG

SEQ ID NO: 171 - protein, 8 aa, Artificial Sequence
  Description of Artificial Sequence Synthetic peptide
QGIDNWLG

SEQ ID NO: 172 - protein, 7 aa, Artificial Sequence
  Description of Artificial Sequence Synthetic peptide
DASNLDT

SEQ ID NO: 173 - protein, 9 aa, Artificial Sequence
  Description of Artificial Sequence Synthetic peptide
QQAKAFPPT

SEQ ID NO: 174 - protein, 118 aa, Artificial Sequence
  Description of Artificial Sequence Synthetic polypeptide
KVQLQQSGTELVKPGASVKVSCKASGYIFTEYIIHWVKQRSGQGLEWIGWLYPESNIIKYNEKFKDKATLTADKSSSTVYMELSRLTSEDSAVYFCTRHDGTNFDYWGQGTTLTVSSA

SEQ ID NO: 175 - protein, 7 aa, Artificial Sequence
  Description of Artificial Sequence Synthetic peptide
GYIFTEY

SEQ ID NO: 176 - protein, 6 aa, Artificial Sequence
  Description of Artificial Sequence Synthetic peptide
YPESNI

SEQ ID NO: 177 - protein, 8 aa, Artificial Sequence
  Description of Artificial Sequence Synthetic peptide
HDGTNFDY

SEQ ID NO: 178 - protein, 112 aa, Artificial Sequence
  Description of Artificial Sequence Synthetic polypeptide
DIVLTQSPASLAVSLGQRATISCRASESVDSYGNSFMHWYQQKPGQPPKLLIYRASNLESGIPARFSGSGSRTDFTLTINPVEADDVATYYCQQSNEDPLTFGAGTKLELKR

SEQ ID NO: 179 - protein, 12 aa, Artificial Sequence
  Description of Artificial Sequence Synthetic peptide
ESVDSYGNSFMH

SEQ ID NO: 180 - protein, 7 aa, Artificial Sequence
  Description of Artificial Sequence Synthetic peptide
RASNLES

SEQ ID NO: 181 - protein, 9 aa, Artificial Sequence
  Description of Artificial Sequence Synthetic peptide
QQSNEDPLT

SEQ ID NO: 182 - protein, 121 aa, Artificial Sequence
  Description of Artificial Sequence Synthetic polypeptide
EIQLQQSGPELVKPGASVKVSCKASGYAFTSYNMYWVKQSHGKSLEWIGYIDPYNGVTSYNQKFKGKATLTVDKSSSTAYMHLNSLTSEDSAVYYCARGGGSIYYAMDYWGQGTSVTVSSA

SEQ ID NO: 183 - protein, 7 aa, Artificial Sequence
  Description of Artificial Sequence Synthetic peptide
GYAFTSY

SEQ ID NO: 184 - protein, 6 aa, Artificial Sequence
  Description of Artificial Sequence Synthetic peptide
DPYNGV

SEQ ID NO: 185 - protein, 11 aa, Artificial Sequence
  Description of Artificial Sequence Synthetic peptide
GGGSIYYAMDY

SEQ ID NO: 186 - protein, 113 aa, Artificial Sequence
  Description of Artificial Sequence Synthetic polypeptide
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELKR

SEQ ID NO: 187 - protein, 13 aa, Artificial Sequence
  Description of Artificial Sequence Synthetic peptide
KSLLHSNGNTYLY

SEQ ID NO: 188 - protein, 7 aa, Artificial Sequence
  Description of Artificial Sequence Synthetic peptide
RMSNLAS

SEQ ID NO: 189 - protein, 9 aa, Artificial Sequence
  Description of Artificial Sequence Synthetic peptide
MQHLEYPLT

SEQ ID NO: 190 - protein, 116 aa, Artificial Sequence
  Description of Artificial Sequence Synthetic polypeptide
EVQLLESGGGLVQPGGSLRLSCAASGFTFSSYAASWVRQAPGKGLEWVSAISGSGGSTYYADSVKGRFTISRDNSKNTLYLQMNSLRAEDTAVYYCAKAHNAFDYWGQGTLVTVSR

SEQ ID NO: 191 - protein, 5 aa, Artificial Sequence
  Description of Artificial Sequence Synthetic peptide
SYAAS

SEQ ID NO: 192 - protein, 16 aa, Artificial Sequence
  Description of Artificial Sequence Synthetic peptide
AISGSGGSTYYADSVK

SEQ ID NO: 193 - protein, 7 aa, Artificial Sequence
  Description of Artificial Sequence Synthetic peptide
AHNAFDY

SEQ ID NO: 194 - protein, 108 aa, Artificial Sequence
  Description of Artificial Sequence Synthetic polypeptide
SELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLG

SEQ ID NO: 195 - protein, 11 aa, Artificial Sequence
  Description of Artificial Sequence Synthetic peptide
QGDSLRSYYAS

SEQ ID NO: 196 - protein, 7 aa, Artificial Sequence
  Description of Artificial Sequence Synthetic peptide
GKNNRPS

SEQ ID NO: 197 - protein, 11 aa, Artificial Sequence
  Description of Artificial Sequence Synthetic peptide
NSSVYTMPPVV

SEQ ID NO: 198 - protein, 119 aa, Artificial Sequence
  Description of Artificial Sequence Synthetic polypeptide
AEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGVLSRYFDYWGQGTLVTVSS

SEQ ID NO: 199 - protein, 8 aa, Artificial Sequence
  Description of Artificial Sequence Synthetic peptide
GFTFDDYG

SEQ ID NO: 200 - protein, 8 aa, Artificial Sequence
  Description of Artificial Sequence Synthetic peptide
INWNGGST

SEQ ID NO: 201 - protein, 11 aa, Artificial Sequence
  Description of Artificial Sequence Synthetic peptide
ARGVLSRYFDY

SEQ ID NO: 202 - protein, 108 aa, Artificial Sequence
  Description of Artificial Sequence Synthetic polypeptide
EIELTQSPATLSLSPGERATLSCRASQSVSSYLAWYQQKPGQAPRLLIYDASNRATGIPARFSGSGSGTDFTLTISSLEPEDFAVYYCQQRSNWPPAFGGGTKVEIKR

SEQ ID NO: 203 - protein, 6 aa, Artificial Sequence
  Description of Artificial Sequence Synthetic peptide
QSVSSY

SEQ ID NO: 204 - protein, 3 aa, Artificial Sequence
  Description of Artificial Sequence Synthetic peptide
DAS

SEQ ID NO: 205 - protein, 9 aa, Artificial Sequence
  Description of Artificial Sequence Synthetic peptide
QQRSNWPPA

SEQ ID NO: 206 - protein, 112 aa, Artificial Sequence
  Description of Artificial Sequence Synthetic polypeptide
QVQLQESGPGLVKPSQTLSLTCTVSGGSITSGYYWNWIRQHPGKGLEWIGYITFDGSNNYNPSLKSRVTISRDTSKNQFSLKLSSVTAADTAVYYCADFDYWGQGTLVTVSS

SEQ ID NO: 207 - protein, 6 aa, Artificial Sequence
  Description of Artificial Sequence Synthetic peptide
SGYYWN

SEQ ID NO: 208 - protein, 16 aa, Artificial Sequence
  Description of Artificial Sequence Synthetic peptide
YITFDGSNNYNPSLKS

SEQ ID NO: 209 - protein, 3 aa, Artificial Sequence
  Description of Artificial Sequence Synthetic peptide
FDY

SEQ ID NO: 210 - protein, 107 aa, Artificial Sequence
  Description of Artificial Sequence Synthetic polypeptide
DIQMTQSPSSLSASVGDRVTITCRASQGIRNYLNWYQQKPGKAPKLLIYYTSSLHSGVPSRFSGSGSGTDYTLTISSLQPEDFATYYCQQYSALPWTFGQGTKVEIK

SEQ ID NO: 211 - protein, 11 aa, Artificial Sequence
  Description of Artificial Sequence Synthetic peptide
RASQGIRNYLN

SEQ ID NO: 212 - protein, 7 aa, Artificial Sequence
  Description of Artificial Sequence Synthetic peptide
YTSSLHS

SEQ ID NO: 213 - protein, 9 aa, Artificial Sequence
  Description of Artificial Sequence Synthetic peptide
QQYSALPWT

SEQ ID NO: 214 - protein, 116 aa, Artificial Sequence
  Description of Artificial Sequence Synthetic polypeptide
QAYLQQSGPELVKAGASVKMSCKASGYSFIEYTINWVKQSHGKSLEWIGNIDPYYGTTYYNQMFTGKATLTVDQSSNTAYMQLKSLTSEDSAVYFCARGSAWFPYWGQGTLVTVSA

SEQ ID NO: 215 - protein, 11 aa, Artificial Sequence
  Description of Artificial Sequence Synthetic peptide
GYSFIEYTINW

SEQ ID NO: 216 - protein, 17 aa, Artificial Sequence
  Description of Artificial Sequence Synthetic peptide
GNIDPYYGTTYYNQMFT

SEQ ID NO: 217 - protein, 9 aa, Artificial Sequence
  Description of Artificial Sequence Synthetic peptide
ARGSAWFPY

SEQ ID NO: 218 - protein, 106 aa, Artificial Sequence
  Description of Artificial Sequence Synthetic polypeptide
DIVMSQSPAIMSASPGEKVTMTCSASSSVTYMYWYQQKPGSSPRLLIYDTSNLASGVPVRFSGSGSGTSYSLTISRMEAEDTATYYCQQWSNYPLTFGAGTKLELK

SEQ ID NO: 219 - protein, 10 aa, Artificial Sequence
  Description of Artificial Sequence Synthetic peptide
SASSSVTYMY

SEQ ID NO: 220 - protein, 7 aa, Artificial Sequence
  Description of Artificial Sequence Synthetic peptide
DTSNLAS

SEQ ID NO: 221 - protein, 9 aa, Artificial Sequence
  Description of Artificial Sequence Synthetic peptide
QQWSNYPLT

SEQ ID NO: 222 - protein, 115 aa, Artificial Sequence
  Description of Artificial Sequence Synthetic polypeptide
QVQLQQSRAELVMPGASVKMSCKTSGYTFSDYWVHWVRQRPGQGLEWIGAIDGSDTFNDYSQKFKGRATLTVDESSSTVYMQLSSLTSEDSAVYYCARGGLLQYWGQGTTLTVSS

SEQ ID NO: 223 - protein, 11 aa, Artificial Sequence
  Description of Artificial Sequence Synthetic peptide
GYTFSDYWVHW

SEQ ID NO: 224 - protein, 17 aa, Artificial Sequence
  Description of Artificial Sequence Synthetic peptide
GAIDGSDTFNDYSQKFK

SEQ ID NO: 225 - protein, 8 aa, Artificial Sequence
  Description of Artificial Sequence Synthetic peptide
ARGGLLQY

SEQ ID NO: 226 - protein, 107 aa, Artificial Sequence
  Description of Artificial Sequence Synthetic polypeptide
DIQLTQSPASLSASVGETVTITCRASENIYSYLAWYQQKQGKSPQLLVYNTKTLGEGVPSRFSGSGSGTQFSLKINSLQPEDFGSYYCQHHYGTPFTFGSGTKLEIK

SEQ ID NO: 227 - protein, 11 aa, Artificial Sequence
  Description of Artificial Sequence Synthetic peptide
RASENIYSYLA

SEQ ID NO: 228 - protein, 7 aa, Artificial Sequence
  Description of Artificial Sequence Synthetic peptide
NTKTLGE

SEQ ID NO: 229 - protein, 9 aa, Artificial Sequence
  Description of Artificial Sequence Synthetic peptide
QHHYGTPFT

SEQ ID NO: 230 - protein, 125 aa, Artificial Sequence
  Description of Artificial Sequence Synthetic polypeptide
EVQLVESGGGLVRPGGSLRLSCAASGFTFSNYDMHWVRQATGKGLEWVSAITAAGDIYYPGSVKGRFTISRENAKNSLYLQMNSLRAGDTAVYYCARGRYSGSGSYYNDWFDPWGQGTLVTVSSA

SEQ ID NO: 231 - protein, 7 aa, Artificial Sequence
  Description of Artificial Sequence Synthetic peptide
GFTFSNY

SEQ ID NO: 232 - protein, 5 aa, Artificial Sequence
  Description of Artificial Sequence Synthetic peptide
TAAGD

SEQ ID NO: 233 - protein, 16 aa, Artificial Sequence
  Description of Artificial Sequence Synthetic peptide
GRYSGSGSYYNDWFDP

SEQ ID NO: 234 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKR

SEQ ID NO: 235 - protein, 8 aa, Artificial Sequence
  Description of Artificial Sequence Synthetic peptide
QSVSSYLA

SEQ ID NO: 236 - protein, 7 aa, Artificial Sequence
  Description of Artificial Sequence Synthetic peptide
DASNRAT

SEQ ID NO: 237 - protein, 9 aa, Artificial Sequence
  Description of Artificial Sequence Synthetic peptide
QQRSNWPLT

SEQ ID NO: 238 - protein, 123 aa, Artificial Sequence
  Description of Artificial Sequence Synthetic polypeptide
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSA

SEQ ID NO: 239 - protein, 7 aa, Artificial Sequence
  Description of Artificial Sequence Synthetic peptide
GYTFTSY

SEQ ID NO: 240 - protein, 6 aa, Artificial Sequence
  Description of Artificial Sequence Synthetic peptide
YPGDGS

SEQ ID NO: 241 - protein, 13 aa, Artificial Sequence
  Description of Artificial Sequence Synthetic peptide
RGEYGNYEGAMDY

SEQ ID NO: 242 - protein, 112 aa, Artificial Sequence
  Description of Artificial Sequence Synthetic polypeptide
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 243 - protein, 15 aa, Artificial Sequence
  Description of Artificial Sequence Synthetic peptide
KASQSVDYDGHSYMN

SEQ ID NO: 244 - protein, 7 aa, Artificial Sequence
  Description of Artificial Sequence Synthetic peptide
AASNLES

SEQ ID NO: 245 - protein, 9 aa, Artificial Sequence
  Description of Artificial Sequence Synthetic peptide
QQSDENPLT

SEQ ID NO: 246 - protein, 118 aa, Artificial Sequence
  Description of Artificial Sequence Synthetic polypeptide
QVQLQQSGSELKKPGASVKISCKASGYSFTDYIILWVRQNPGKGLEWIGHIDPYYGSSNYNLKFKGRVTITADQSTTTAYMELSSLRSEDTAVYYCGRSKRDYFDYWGQGTTLTVSSA

SEQ ID NO: 247 - protein, 7 aa, Artificial Sequence
  Description of Artificial Sequence Synthetic peptide
GYSFTDY

SEQ ID NO: 248 - protein, 6 aa, Artificial Sequence
  Description of Artificial Sequence Synthetic peptide
DPYYGS

SEQ ID NO: 249 - protein, 8 aa, Artificial Sequence
  Description of Artificial Sequence Synthetic peptide
SKRDYFDY

SEQ ID NO: 250 - protein, 108 aa, Artificial Sequence
  Description of Artificial Sequence Synthetic polypeptide
DIQMTQSPSSLSASIGDRVTITCKASQDINSYLSWFQQKPGKAPKLLIYRANRLVDGVPSRFSGSGSGTDYTLTISSLQPEDFAVYYCLQYDEFPYTFGGGTKLEIKR

SEQ ID NO: 251 - protein, 8 aa, Artificial Sequence
  Description of Artificial Sequence Synthetic peptide
QDINSYLS

SEQ ID NO: 252 - protein, 7 aa, Artificial Sequence
  Description of Artificial Sequence Synthetic peptide
RANRLVD

SEQ ID NO: 253 - protein, 9 aa, Artificial Sequence
  Description of Artificial Sequence Synthetic peptide
LQYDEFPYT

SEQ ID NO: 254 - protein, 118 aa, Artificial Sequence
  Description of Artificial Sequence Synthetic polypeptide
EVQLVETGGGVVQPGRSLRLSCAASGFTFSSYAMHWVRQAPGKGLEWVAVISYSGSNKYYADSVKGRFTISRDNSKNTLYLQMNSLRAEDTAVYYCARHEWGFGFDYWGQGTTVTVSS

SEQ ID NO: 255 - protein, 8 aa, Artificial Sequence
  Description of Artificial Sequence Synthetic peptide
GFTFSSYA

SEQ ID NO: 256 - protein, 8 aa, Artificial Sequence
  Description of Artificial Sequence Synthetic peptide
ISYSGSNK

SEQ ID NO: 257 - protein, 11 aa, Artificial Sequence
  Description of Artificial Sequence Synthetic peptide
ARHEWGFGFDY

SEQ ID NO: 258 - protein, 119 aa, Artificial Sequence
  Description of Artificial Sequence Synthetic polypeptide
DVVMTQSPLSLPVTLGQPASISCRSSQSLVYGDGNTYLNWFQQRPGQSPRRLIYKVSNRDSGVPDRFSGSGSGTEFTLKISRVEAEDVGVYFCMQGTYWPGTFGGGTKLEIKRTVAAPS

SEQ ID NO: 259 - protein, 11 aa, Artificial Sequence
  Description of Artificial Sequence Synthetic peptide
QSLVYGDGNTY

SEQ ID NO: 260 - protein, 3 aa, Artificial Sequence
  Description of Artificial Sequence Synthetic peptide
KVS

SEQ ID NO: 261 - protein, 7 aa, Artificial Sequence
  Description of Artificial Sequence Synthetic peptide
MQGTYWP

SEQ ID NO: 262 - protein, 113 aa, Artificial Sequence
  Description of Artificial Sequence Synthetic polypeptide
QVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSA

SEQ ID NO: 263 - protein, 10 aa, Artificial Sequence
  Description of Artificial Sequence Synthetic peptide
GYTFTNYDIH

SEQ ID NO: 264 - protein, 17 aa, Artificial Sequence
  Description of Artificial Sequence Synthetic peptide
WIYPGDGSTKYNEKFKG

SEQ ID NO: 265 - protein, 4 aa, Artificial Sequence
  Description of Artificial Sequence Synthetic peptide
EWAY

SEQ ID NO: 266 - protein, 107 aa, Artificial Sequence
  Description of Artificial Sequence Synthetic polypeptide
DIQMTQSPSSLSASLGERVSLTCRASQDIGGNLYWLQQGPDGTIKRLIYATSSLDSGVPKRFSGSRSGSDYSLTISSLESEDFVDYYCLQYSSSPWTFGGGTKMEIK

SEQ ID NO: 267 - protein, 11 aa, Artificial Sequence
  Description of Artificial Sequence Synthetic peptide
RASQDIGGNLY

SEQ ID NO: 268 - protein, 7 aa, Artificial Sequence
  Description of Artificial Sequence Synthetic peptide
ATSSLDS

SEQ ID NO: 269 - protein, 9 aa, Artificial Sequence
  Description of Artificial Sequence Synthetic peptide
LQYSSSPWT

SEQ ID NO: 270 - protein, 122 aa, Artificial Sequence
  Description of Artificial Sequence Synthetic polypeptide
EVQLLESGGGLVQPGGSLRLSCAASGFTFSEYTMSWVRQAPGKGLEWVSRIYSSGGHTEYADSVKGRFTISRDNSKNTLYLQMNSLRAEDTAVYYCAKGSGYYHYYYGMDVWGQGTTVTVSS

SEQ ID NO: 271 - protein, 5 aa, Artificial Sequence
  Description of Artificial Sequence Synthetic peptide
EYTMS

SEQ ID NO: 272 - protein, 17 aa, Artificial Sequence
  Description of Artificial Sequence Synthetic peptide
RIYSSGGHTEYADSVKG

SEQ ID NO: 273 - protein, 13 aa, Artificial Sequence
  Description of Artificial Sequence Synthetic peptide
GSGYYHYYYGMDV

SEQ ID NO: 274 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 275 - protein, 11 aa, Artificial Sequence
  Description of Artificial Sequence Synthetic peptide
RASQSISSYLN

SEQ ID NO: 276 - protein, 7 aa, Artificial Sequence
  Description of Artificial Sequence Synthetic peptide
AASSLQS

SEQ ID NO: 277 - protein, 9 aa, Artificial Sequence
  Description of Artificial Sequence Synthetic peptide
QQSYSTPIT

SEQ ID NO: 278 - protein, 5 aa, Artificial Sequence
  Description of Artificial Sequence Synthetic peptide
GYYMH

SEQ ID NO: 279 - protein, 15 aa, Artificial Sequence
  Description of Artificial Sequence Synthetic peptide
INPSGGTTRLAQKFQ

SEQ ID NO: 280 - protein, 12 aa, Artificial Sequence
  Description of Artificial Sequence Synthetic peptide
EAHSSGSYFFDY

SEQ ID NO: 281 - protein, 11 aa, Artificial Sequence
  Description of Artificial Sequence Synthetic peptide
RASQSISTWLA

SEQ ID NO: 282 - protein, 7 aa, Artificial Sequence
  Description of Artificial Sequence Synthetic peptide
QASTLTS

SEQ ID NO: 283 - protein, 10 aa, Artificial Sequence
  Description of Artificial Sequence Synthetic peptide
QEYNSYSPWA

SEQ ID NO: 284 - protein, 119 aa, Artificial Sequence
  Description of Artificial Sequence Synthetic polypeptide
QVQLVQSGAEVKKPGASVKVSCKASGYTFTNYRIEWVRQAPGQGLEWMGEILPRGGNTNYNEKFKGRVTFTADTSTSTAYMELRSLRSDDTAVYYCARDDGYYGRFAYWGQGTLVTVSS

SEQ ID NO: 285 - protein, 5 aa, Artificial Sequence
  Description of Artificial Sequence Synthetic peptide
NYRIE

SEQ ID NO: 286 - protein, 17 aa, Artificial Sequence
  Description of Artificial Sequence Synthetic peptide
EILPRGGNTNYNEKFKG

SEQ ID NO: 287 - protein, 10 aa, Artificial Sequence
  Description of Artificial Sequence Synthetic peptide
DDGYYGRFAY

SEQ ID NO: 288 - protein, 107 aa, Artificial Sequence
  Description of Artificial Sequence Synthetic polypeptide
DIQMTQSPSSLSASVGDRVTITCRASQDISNYLNWYQQKPGKAPKLLIYYTSRLHSGVPSRFSGSGSGTDYTLTISSLQPEDFATYYCQQGNTLPPTFGGGTKVEIK

SEQ ID NO: 289 - protein, 11 aa, Artificial Sequence
  Description of Artificial Sequence Synthetic peptide
RASQDISNYLN

SEQ ID NO: 290 - protein, 7 aa, Artificial Sequence
  Description of Artificial Sequence Synthetic peptide
YTSRLHS

SEQ ID NO: 291 - protein, 9 aa, Artificial Sequence
  Description of Artificial Sequence Synthetic peptide
QQGNTLPPT

SEQ ID NO: 292 - protein, 119 aa, Artificial Sequence; identical to SEQ ID NO: 284
  Description of Artificial Sequence Synthetic polypeptide

SEQ ID NO: 293 - protein, 5 aa, Artificial Sequence
  Description of Artificial Sequence Synthetic peptide
NYRIE

SEQ ID NO: 294 - protein, 17 aa, Artificial Sequence
  Description of Artificial Sequence Synthetic peptide
EILPRGGNTNYNEKFKG

SEQ ID NO: 295 - protein, 10 aa, Artificial Sequence
  Description of Artificial Sequence Synthetic peptide
DDGYYGRFAY

SEQ ID NO: 296 - protein, 107 aa, Artificial Sequence; identical to SEQ ID NO: 288
  Description of Artificial Sequence Synthetic polypeptide

SEQ ID NO: 297 - protein, 11 aa, Artificial Sequence
  Description of Artificial Sequence Synthetic peptide
RASQDISNYLN

SEQ ID NO: 298 - protein, 7 aa, Artificial Sequence
  Description of Artificial Sequence Synthetic peptide
YTSRLHS

SEQ ID NO: 299 - protein, 9 aa, Artificial Sequence
  Description of Artificial Sequence Synthetic peptide
QQGNTLPPT

SEQ ID NO: 300 - protein, 120 aa, Artificial Sequence
  Description of Artificial Sequence Synthetic polypeptide
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 301 - protein, 7 aa, Artificial Sequence
  Description of Artificial Sequence Synthetic peptide
GYTFSTF

SEQ ID NO: 302 - protein, 6 aa, Artificial Sequence
  Description of Artificial Sequence Synthetic peptide
HTYAGV

SEQ ID NO: 303 - protein, 11 aa, Artificial Sequence
  Description of Artificial Sequence Synthetic peptide
RSDNYRYFFDY

SEQ ID NO: 304 - protein, 107 aa, Artificial Sequence
  Description of Artificial Sequence Synthetic polypeptide
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 305 - protein, 11 aa, Artificial Sequence
  Description of Artificial Sequence Synthetic peptide
RASQDIRNYLN

SEQ ID NO: 306 - protein, 7 aa, Artificial Sequence
  Description of Artificial Sequence Synthetic peptide
YTSRLHS

SEQ ID NO: 307 - protein, 9 aa, Artificial Sequence
  Description of Artificial Sequence Synthetic peptide
QQGHTLPPT

SEQ ID NO: 308 - protein, 122 aa, Artificial Sequence
  Description of Artificial Sequence Synthetic polypeptide
QVQLQQWGAGLLKPSETLSLTCAVYGGSFSGYYWSWIRQPPGKGLEWIGEIHHSGGANYNPSLKSRVTISVDTSKNQFSLKLSSVTAADTAVYYCARGQGKNWHYDYFDYWGQGTLVTVSSA

SEQ ID NO: 309 - protein, 5 aa, Artificial Sequence
  Description of Artificial Sequence Synthetic peptide
GYYWS

SEQ ID NO: 310 - protein, 16 aa, Artificial Sequence
  Description of Artificial Sequence Synthetic peptide
EIHHSGGANYNPSLKS

SEQ ID NO: 311 - protein, 13 aa, Artificial Sequence
  Description of Artificial Sequence Synthetic peptide
GQGKNWHYDYFDY

SEQ ID NO: 312 - protein, 107 aa, Artificial Sequence
  Description of Artificial Sequence Synthetic polypeptide
DIQMTQSPSTLSASVGDRVTITCRASQSIRSWLAWYQQKPGKAPKLLIYKASILKIGVPSRFSGSGSGTEFTLTISSLQPDDFATYYCQQYYSYSRTFGQGTKVEIK

SEQ ID NO: 313 - protein, 11 aa, Artificial Sequence
  Description of Artificial Sequence Synthetic peptide
RASQSIRSWLA

SEQ ID NO: 314 - protein, 7 aa, Artificial Sequence
  Description of Artificial Sequence Synthetic peptide
KASILKI

SEQ ID NO: 315 - protein, 9 aa, Artificial Sequence
  Description of Artificial Sequence Synthetic peptide
QQYYSYSRT

SEQ ID NO: 316 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 317 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 318 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 319 - protein, 1299 aa, Homo sapiens
MSLQEMFRFPMGLLLGSVLLVASAPATLEPPGCSNKEQQVTVSHTYKIDVPKSALVQVDADPQPLSDDGASLLALGEAREEQNIIFRHNIRLQTPQKDCELAGSVQDLLARVKKLEEEMVEMKEQCSAQRCCQGVTDLSRHCSGHGTFSLETCSCHCEEGREGPACERLACPGACSGHGRCVDGRCLCHEPYVGADCGYPACPENCSGHGECVRGVCQCHEDFMSEDCSEKRCPGDCSGHGFCDTGECYCEEGFTGLDCAQVVTPQGLQLLKNTEDSLLVSWEPSSQVDHYLLSYYPLGKELSGKQIQVPKEQHSYEILGLLPGTKYIVTLRNVKNEVSSSPQHLLATTDLAVLGTAWVTDETENSLDVEWENPSTEVDYYKLRYGPMTGQEVAEVTVPKSSDPKSRYDITGLHPGTEYKITVVPMRGELEGKPILLNGRTEIDSPTNVVTDRVTEDTATVSWDPVQAVIDKYVVRYTSADGDTKEMAVHKDESSTVLTGLKPGEAYKVYVWAERGNQGSKKADTNALTEIDSPANLVTDRVTENTATISWDPVQATIDKYVVRYTSADDQETREVLVGKEQSSTVLTGLRPGVEYTVHVWAQKGDRESKKADTNAPTDIDSPKNLVTDRVTENMATVSWDPVQAAIDKYVVRYTSAGGETREVPVGKEQSSTVLTGLRPGMEYMVHVWAQKGDQESKKADTKAQTDIDSPQNLVTDRVTENMATVSWDPVRATIDRYVVRYTSAKDGETREVPVGKEQSSTVLTGLRPGVEYTVHVWAQKGAQESKKADTKAQTDIDSPQNLVTDWVTENTATVSWDPVQATIDRYVVHYTSANGETREVPVGKEQSSTVLTGLRPGMEYTVHVWAQKGNQESKKADTKAQTEIDGPKNLVTDWVTENMATVSWDPVQATIDKYMVRYTSADGETREVPVGKEHSSTVLTGLRPGMEYMVHVWAQKGAQESKKADTKAQTELDPPRNLRPSAVTQSGGILTWTPPSAQIHGYILTYQFPDGTVKEMQLGREDQRFALQGLEQGATYPVSLVAFKGGRRSRNVSTTLSTVGARFPHPSDCSQVQQNSNAASGLYTIYLHGDASRPLQVYCDMETDGGGWIVFQRRNTGQLDFFKRWRSYVEGFGDPMKEFWLGLDKLHNLTTGTPARYEVRVDLQTANESAYAIYDFFQVASSKERYKLTVGKYRGTAGDALTYHNGWKFTTFDRDNDIALSNCALTHHGGWWYKNCHLANPNGRYGETKHSEGVNWEPWKGHEFSIPYVELKIRPHGYSREPVLGRKKRTLRGRLRTF

SEQ ID NO: 320 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 321 - protein, 318 aa, Homo sapiens
MAAQGCAASRLLQLLLQLLLLLLLLAAGGARARWRGEGTSAHLRDIFLGRCAEYRALLSPEQRNKNCTAIWEAFKVALDKDPCSVLPSDYDLFINLSRHSIPRDKSLFWENSHLLVNSFADNTRRFMPLSDVLYGRVADFLSWCRQKNDSGLDYQSCPTSEDCENNPVDSFWKRASIQYSKDSSGVIHVMLNGSEPTGAYPIKGFFADYEIPNLQKEKITRIEIWVMHEIGGPNVESCGEGSMKVLEKRLKDMGFQYSCINDYRPVKLLQCVDHSTHPDCALKSAAAATQRKAPSLYTEQRAGLIIPLFLVLASRTQL

SEQ ID NO: 322 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 323 - protein, 278 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

SEQ ID NO: 324 - protein, 1382 aa, Homo sapiens
MATGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 325 - protein, 1130 aa, Homo sapiens
MAAAGQLCLLYLSAGLLSRLGAAFNLDTREDNVIRKYGDPGSLFGFSLAMHWQLQPEDKRLLLVGAPRAEALPLQRANRTGGLYSCDITARGPCTRIEFDNDADPTSESKEDQWMGVTVQSQGPGGKVVTCAHRYEKRQHVNTKQESRDIFGRCYVLSQNLRIEDDMDGGDWSFCDGRLRGHEKFGSCQQGVAATFTKDFHYIVFGAPGTYNWKGIVRVEQKNNTFFDMNIFEDGPYEVGGETEHDESLVPVPANSYLGLLFLTSVSYTDPDQFVYKTRPPREQPDTFPDVMMNSYLGFSLDSGKGIVSKDEITFVSGAPRANHSGAVVLLKRDMKSAHLLPEHIFDGEGLASSFGYDVAVVDLNKDGWQDIVIGAPQYFDRDGEVGGAVYVYMNQQGRWNNVKPIRLNGTKDSMFGIAVKNIGDINQDGYPDIAVGAPYDDLGKVFIYHGSANGINTKPTQVLKGISPYFGYSIAGNMDLDRNSYPDVAVGSLSDSVTIFRSRPVINIQKTITVTPNRIDLRQKTACGAPSGICLQVKSCFEYTANPAGYNPSISIVGTLEAEKERRKSGLSSRVQFRNQGSEPKYTQELTLKRQKQKVCMEETLWLQDNIRDKLRPIPITASVEIQEPSSRRRVNSLPEVLPILNSDEPKTAHIDVHFLKEGCGDDNVCNSNLKLEYKFCTREGNQDKFSYLPIQKGVPELVLKDQKDIALEITVTNSPSNPRNPTKDGDDAHEAKLIATFPDTLTYSAYRELRAFPEKQLSCVANQNGSQADCELGNPFKRNSNVTFYLVLSTTEVTFDTPDLDINLKLETTSNQDNLAPITAKAKVVIELLLSVSGVAKPSQVYFGGTVVGEQAMKSEDEVGSLIEYEFRVINLGKPLTNLGTATLNIQWPKEISNGKWLLYLVKVESKGLEKVTCEPQKEINSLNLTESHNSRKKREITEKQIDDNRKFSLFAERKYQTLNCSVNVNCVNIRCPLRGLDSKASLILRSRLWNSTFLEEYSKLNYLDILMRAFIDVTAAAENIRLPNAGTQVRVTVFPSKTVAQYSGVPWWIILVAILAGILMLALLVFILWKCGFFKRSRYDDSVPRYHAVRIRKEEREIKDEKYIDNLEKKQWITKWNENESYS

SEQ ID NO: 326 - protein, 738 aa, Homo sapiens
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 327 - protein, 738 aa, Homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQVLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 328 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 329 - protein, 249 aa, Artificial Sequence
  Description of Artificial Sequence Synthetic polypeptide
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGCGTKLEIKGGGGSGGGGSGGGGSGGGGSEVQLVESGGGLVQPGGSLRLSCAASGFTFSSFGMHWVRQAPGKCLEWVAYISSDSSAIYYADTVKGRFTISRDNAKNSLYLQMNSLRDEDTAVYYCGRGRENIYYGSRLDYWGQGTTVTVSS

SEQ ID NO: 330 - protein, 477 aa, Artificial Sequence
  Description of Artificial Sequence Synthetic polypeptide
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGCGTKLEIKGGGGSGGGGSGGGGSGGGGSEVQLVESGGGLVQPGGSLRLSCAASGFTFSSFGMHWVRQAPGKCLEWVAYISSDSSAIYYADTVKGRFTISRDNAKNSLYLQMNSLRDEDTAVYYCGRGRENIYYGSRLDYWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 331 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 332 - protein, 214 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 243 aa, Artificial Sequence
  Description of Artificial Sequence Synthetic polypeptide
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGCGTKLEIKGGGGSGGGGSGGGGSGGGGSEVQLQESGPGLVKPSETLSLTCAVTGYSITSGYSWHWIRQFPGNCLEWMGYIHSSGSTNYNPSLKSRISISRDTSKNQFFLKLSSVTAADTAVYYCAGYDDYFEYWGQGTTVTVSS

SEQ ID NO: 334 - protein, 471 aa, Artificial Sequence
  Description of Artificial Sequence Synthetic polypeptide
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGCGTKLEIKGGGGSGGGGSGGGGSGGGGSEVQLQESGPGLVKPSETLSLTCAVTGYSITSGYSWHWIRQFPGNCLEWMGYIHSSGSTNYNPSLKSRISISRDTSKNQFFLKLSSVTAADTAVYYCAGYDDYFEYWGQGTTVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 335 - protein, 248 aa, Artificial Sequence
  Description of Artificial Sequence Synthetic polypeptide
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGCGTKVEIKGGGGSGGGGSGGGGSGGGGSQVQLVQSGAEVKKPGSSVKVSCKASGYTFTNYVMHWVRQAPGQCLEWMGYINPYNDDVKYNEKFKGRVTITADESTSTAYMELSSLRSEDTAVYYCARWGYYGSPLYYFDYWGQGTLVTVSS

SEQ ID NO: 336 - protein, 476 aa, Artificial Sequence
  Description of Artificial Sequence Synthetic polypeptide
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGCGTKVEIKGGGGSGGGGSGGGGSGGGGSQVQLVQSGAEVKKPGSSVKVSCKASGYTFTNYVMHWVRQAPGQCLEWMGYINPYNDDVKYNEKFKGRVTITADESTSTAYMELSSLRSEDTAVYYCARWGYYGSPLYYFDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 337 - protein, 5 aa, Artificial Sequence
  Description of Artificial Sequence Synthetic peptide
SYAIS

SEQ ID NO: 338 - protein, 16 aa, Artificial Sequence
  Description of Artificial Sequence Synthetic peptide
GDSSIRHAYYYYGMDV

SEQ ID NO: 339 - protein, 7 aa, Artificial Sequence
  Description of Artificial Sequence Synthetic peptide
SSSYYWG

SEQ ID NO: 340 - protein, 11 aa, Artificial Sequence
  Description of Artificial Sequence Synthetic peptide
GSDRFHPYFDY

SEQ ID NO: 341 - protein, 5 aa, Artificial Sequence
  Description of Artificial Sequence Synthetic peptide
SYYMH

SEQ ID NO: 342 - protein, 17 aa, Artificial Sequence
  Description of Artificial Sequence Synthetic peptide
GAPNYGDTTHDYYYMDV

SEQ ID NO: 343 - protein, 5 aa, Artificial Sequence
  Description of Artificial Sequence Synthetic peptide
GYYMH

SEQ ID NO: 344 - protein, 15 aa, Artificial Sequence
  Description of Artificial Sequence Synthetic peptide
DTGEYYDTDDHGMDV

SEQ ID NO: 345 - protein, 5 aa, Artificial Sequence
  Description of Artificial Sequence Synthetic peptide
SYAMS

SEQ ID NO: 346 - protein, 12 aa, Artificial Sequence
  Description of Artificial Sequence Synthetic peptide
DGGYYDSGAGDY

SEQ ID NO: 347 - protein, 5 aa, Artificial Sequence
  Description of Artificial Sequence Synthetic peptide
SYSMN

SEQ ID NO: 348 - protein, 13 aa, Artificial Sequence
  Description of Artificial Sequence Synthetic peptide
GAPMGAAAGWFDP

SEQ ID NO: 349 - protein, 5 aa, Artificial Sequence
  Description of Artificial Sequence Synthetic peptide
SYYMH

SEQ ID NO: 350 - protein, 16 aa, Artificial Sequence
  Description of Artificial Sequence Synthetic peptide
EGAGFAYGMDYYYMDV

SEQ ID NO: 351 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 352 - protein, 13 aa, Artificial Sequence
  Description of Artificial Sequence Synthetic peptide
GAPIGAAAGWFDP

SEQ ID NO: 353 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 354 - protein, 15 aa, Artificial Sequence
  Description of Artificial Sequence Synthetic peptide
ARGAPIGAAAGWFDP

SEQ ID NO: 355 - protein, 13 aa, Artificial Sequence
  Description of Artificial Sequence Synthetic peptide
GAPQGAAAGWFDP

SEQ ID NO: 356 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 357 - protein, 15 aa, Artificial Sequence
  Description of Artificial Sequence Synthetic peptide
ARGAPLGAAAGWFDP

SEQ ID NO: 358 - protein, 13 aa, Artificial Sequence
  Description of Artificial Sequence Synthetic peptide
GAPLGAAAGWFDP

SEQ ID NO: 359 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 360 - protein, 15 aa, Artificial Sequence
  Description of Artificial Sequence Synthetic peptide
ARGAPFGAAAGWFDP

SEQ ID NO: 361 - protein, 13 aa, Artificial Sequence
  Description of Artificial Sequence Synthetic peptide
GAPFGAAAGWFDP

SEQ ID NO: 362 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 363 - protein, 15 aa, Artificial Sequence
  Description of Artificial Sequence Synthetic peptide
ARGAPVGAAAGWFDP

SEQ ID NO: 364 - protein, 13 aa, Artificial Sequence
  Description of Artificial Sequence Synthetic peptide
GAPVGAAAGWFDP

SEQ ID NO: 365 - protein, 122 aa, Artificial Sequence
  Description of Artificial Sequence Synthetic polypeptide
  MOD_RES (102)..(102): Met, Leu, Ile, Val, Gln or Phe
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 366 - protein, 15 aa, Artificial Sequence
  Description of Artificial Sequence Synthetic peptide
  MOD_RES (6)..(6): Met, Leu, Ile, Val, Gln or Phe
ARGAPXGAAAGWFDP

SEQ ID NO: 367 - protein, 13 aa, Artificial Sequence
  Description of Artificial Sequence Synthetic peptide
  MOD_RES (4)..(4): Met, Leu, Ile, Val, Gln or Phe
GAPXGAAAGWFDP

SEQ ID NO: 368 - protein, 11 aa, Artificial Sequence
  Description of Artificial Sequence Synthetic peptide
KASQNVDTNVA

SEQ ID NO: 369 - protein, 122 aa, Artificial Sequence
  Description of Artificial Sequence Synthetic polypeptide
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 370 - protein, 106 aa, Artificial Sequence
  Description of Artificial Sequence Synthetic polypeptide
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 371 - protein, 7 aa, Artificial Sequence
  Description of Artificial Sequence Synthetic peptide
GYTFTNY

SEQ ID NO: 372 - protein, 6 aa, Artificial Sequence
  Description of Artificial Sequence Synthetic peptide
NPYNDD

SEQ ID NO: 373 - protein, 13 aa, Artificial Sequence
  Description of Artificial Sequence Synthetic peptide
WGYYGSPLYYFDY

SEQ ID NO: 374 - protein, 10 aa, Artificial Sequence
  Description of Artificial Sequence Synthetic peptide
RASSRLIYMH

SEQ ID NO: 375 - protein, 7 aa, Artificial Sequence
  Description of Artificial Sequence Synthetic peptide
ATSNLAS

SEQ ID NO: 376 - protein, 9 aa, Artificial Sequence
  Description of Artificial Sequence Synthetic peptide
QQWNSNPPT

SEQ ID NO: 377 - protein, 116 aa, Artificial Sequence
  Description of Artificial Sequence Synthetic polypeptide
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 378 - protein, 107 aa, Artificial Sequence
  Description of Artificial Sequence Synthetic polypeptide
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 379 - protein, 8 aa, Artificial Sequence
  Description of Artificial Sequence Synthetic peptide
GYSITSGY

SEQ ID NO: 380 - protein, 5 aa, Artificial Sequence
  Description of Artificial Sequence Synthetic peptide
HSSGS

SEQ ID NO: 381 - protein, 7 aa, Artificial Sequence
  Description of Artificial Sequence Synthetic peptide
YDDYFEY

SEQ ID NO: 382 - protein, 11 aa, Artificial Sequence
  Description of Artificial Sequence Synthetic peptide
KASQNVGFNVA

SEQ ID NO: 383 - protein, 7 aa, Artificial Sequence
  Description of Artificial Sequence Synthetic peptide
SASYRYS

SEQ ID NO: 384 - protein, 9 aa, Artificial Sequence
  Description of Artificial Sequence Synthetic peptide
QQYNWYPFT

SEQ ID NO: 385 - protein, 15 aa, Artificial Sequence
  Description of Artificial Sequence Synthetic peptide
ARGAPQGAAAGWFDP

SEQ ID NO: 386 - protein, 123 aa, Artificial Sequence
  Description of Artificial Sequence Synthetic polypeptide
EVQLVESGGGLVQPGGSLRLSCAASGFTFSSFGMHWVRQAPGKCLEWVAYISSDSSAIYYADTVKGRFTISRDNAKNSLYLQMNSLRDEDTAVYYCGRGRENIYYGSRLDYWGQGTTVTVSSA

SEQ ID NO: 387 - protein, 107 aa, Artificial Sequence
  Description of Artificial Sequence Synthetic polypeptide
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGCGTKLEIK

SEQ ID NO: 388 - protein, 122 aa, Artificial Sequence
  Description of Artificial Sequence Synthetic polypeptide
QVQLVQSGAEVKKPGSSVKVSCKASGYTFTNYVMHWVRQAPGQCLEWMGYINPYNDDVKYNEKFKGRVTITADESTSTAYMELSSLRSEDTAVYYCARWGYYGSPLYYFDYWGQGTLVTVSS

SEQ ID NO: 389 - protein, 106 aa, Artificial Sequence
  Description of Artificial Sequence Synthetic polypeptide
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGCGTKVEIK

SEQ ID NO: 390 - protein, 116 aa, Artificial Sequence
  Description of Artificial Sequence Synthetic polypeptide
EVQLQESGPGLVKPSETLSLTCAVTGYSITSGYSWHWIRQFPGNCLEWMGYIHSSGSTNYNPSLKSRISISRDTSKNQFFLKLSSVTAADTAVYYCAGYDDYFEYWGQGTTVTVSS

SEQ ID NO: 391 - protein, 107 aa, Artificial Sequence
  Description of Artificial Sequence Synthetic polypeptide
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGCGTKLEIK

(19) United States
(12) Patent Application Publication
(10) Pub. No.: US 2021/0206859 A1
(43) Pub. Date: Jul. 8, 2021

(54) PROTEINS BINDING NKG2D, CD16 AND A TUMOR-ASSOCIATED ANTIGEN

(71) Applicant: Dragonfly Therapeutics, Inc., Waltham, MA (US)

(72) Inventors: Gregory P. Chang, Medford, MA (US); Ann F. Cheung, Lincoln, MA (US); Jinyan Du, Waltham, MA (US); Asya Grinberg, Lexington, MA (US); William Haney, Wayland, MA (US); Bradley M. Lunde, Lebanon, NH (US); Bianka Prinz, Lebanon, NH (US)

(21) Appl. No.: 17/265,879

(22) PCT Filed: Aug. 8, 2019
(86) PCT No.: PCT/US2019/045723

§ 371 Date: Feb. 4, 2021

Related U.S. Application Data
(60) Provisional application No. 62/716,109, filed on Aug. 8, 2018.
(60) Provisional application No. 62/716,113, filed on Aug. 8, 2018.
(60) Provisional application No. 62/716,106, filed on Aug. 8, 2018.

(51) Int. Cl. C07K 16/28; A61P 35/00

(52) U.S. Cl. CPC C07K 16/2851; C07K 16/2827; C07K 16/283; C07K 16/2803; C07K 16/2863; C07K 2317/73; A61P 35/00; C07K 2317/55; C07K 2317/622; C07K 2317/31; C07K 16/2896

(57) ABSTRACT

Multi-specific binding proteins that bind NKG2D receptor, CD 16, and a tumor-associated antigen (e.g, B7-H3, L1CAM, FLT1, KDR, TNC, TNN, CSPG4, BST1, SELF, CD200, INSR (HHF5), ITGA6, MELTF, PECAM1, or SLC1A5) are described, as well as pharmaceutical compositions and therapeutic methods useful for the treatment of cancer.

### CROSS-REFERENCE TO RELATED APPLICATIONS

This application claims the benefit of and priority to U.S. Provisional Patent Application No. 62/716,106, filed Aug. 8, 2018; U.S. Provisional Patent Application No. 62/716,109, filed Aug. 8, 2018; U.S. Provisional Patent Application No. 62/716,113, filed Aug. 8, 2018; the disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Aug. 7, 2019, is named DFY-059WO_ST25.txt and is 367,901 bytes in size.

### FIELD OF THE INVENTION

The invention relates to multi-specific binding proteins that bind to NKG2D, CD16, and a tumor-associated antigen (e.g., B7-H3, L1CAM, FLT1, KDR, TNC, TNN, CSPG4, BST1, SELP, CD200, INSR (HHF5), ITGA6, MELTF, PECAM1, or SLC1A5).

### BACKGROUND

Cancer continues to be a significant health problem despite the substantial research efforts and scientific advances reported in the literature for treating the disease. Some of the most frequently diagnosed cancers include prostate cancer, breast cancer, lung cancer, and colorectal cancer. Prostate cancer is the most common form of cancer in men. Breast cancer remains a leading cause of death in women. Blood and bone marrow cancers are also frequently diagnosed cancer types, including multiple myelomas, leukemia, and lymphomas. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals.

B7-H3, also known as CD276, is a major glycoprotein expressed on antigen-presenting cells (APC). It acts as a co-inhibitory molecule of T-cell activity, together with immune checkpoints, such as PD-1 and CTLA4. B7-H3 is expressed largely on tumor and tumor-associated cells, for example, lung, breast, brain, kidney, and prostate cancers. B7-H3 appears to be widely associated with different proteins that contribute to cancer migration, invasion, and angiogenesis (See, Castellanos et al., Am J Clin Exp Immunol. 2017; 6(4): 66-75).

L1 cell adhesion molecule (L1CAM) is a 200-220 kDa transmembrane glycoprotein of the immunoglobulin (Ig) superfamily, and is composed of six Ig-like domains and five fibronectin Type III repeats followed by a transmembrane region and a highly conserved cytoplasmic tail. It is the prototype member of the L1-family of closely related neural cell adhesion molecules (CAMs), and plays an essential role in neural cell adhesion and migration. In addition, L1CAM is found to be associated with progression of human cancers, including poor prognosis, tumor progression and metastasis to lymph nodes. L1CAM is expressed in many cancers, for example, in bladder cancer, renal cancer, breast cancer, cervical cancer, sarcoma, lung cancer, head and neck cancer, glioblastoma, neuroblastoma, melanoma, ovarian cancer, endometrial cancer, esophageal cancer, gastric cancer, gastrointestinal stromal tumor (GIST), cholangiocarcinoma, colorectal cancer, pancreatic cancer, and prostate cancer (See, Altevogt et al., Int. J. Cancer. 2016; 138: 1565-1576).

Vascular endothelial growth factor receptor 1 (VEGFR1), also named FLT1, is a receptor tyrosine kinase that binds to VEGF-A, VEGF-B, and placental growth factor (PGF). It is expressed in vascular endothelial cells, placental trophoblast cells, and peripheral blood monocytes, and plays an important role in angiogenesis and vasculogenesis. A full-length transmembrane receptor isoform and shortened, soluble isoforms of FLT1 have been found. The soluble isoforms are associated with the onset of pre-eclampsia.

Kinase insert domain receptor (KDR) is a receptor tyrosine kinase that binds to VEGF-A, VEGF-C, and VEGF-D. It is expressed in vascular endothelial cells, and plays an important role in VEGF-induced endothelial proliferation, survival, migration, tubular morphogenesis and sprouting. Mutations of KDR are implicated in infantile capillary hemangiomas.

Tenascin C (TNC) is an extracellular matrix protein having a homohexameric structure with disulfide-linked subunits. TNC has many extracellular binding partners, including matrix components, soluble factors and pathogens, and cell surface receptors. TNC protein synthesis is tightly regulated, with widespread protein distribution in embryonic tissues, but restricted distribution in adult tissues. TNC is also expressed during chronic inflammation and cancer.

Tenascin N (TNN) is a homohexameric extracellular matrix protein. TNN is not detected in normal adult mammary tissues or brain, but is expressed in breast tumors and brain tumors, such as glioblastomas, astrocytomas and oligodendrogliomas. In brain tumors, it is detected around the endothelial cell layer of the blood vessels.

Chondroitin sulfate proteoglycan 4 (CSPG4) is an integral membrane chondroitin sulfate proteoglycan expressed by human malignant melanoma cells. It binds to growth factors and extracellular matrix proteases through its extracellular N-terminus. CSPG4 plays a role in stabilizing cell-substratum interactions during early events of melanoma cell spreading on endothelial basement membranes.

Bone marrow stromal cell antigen 1 (BST1) is glycosylphosphatidylinositol-anchored enzyme for the synthesis of second messengers cyclic ADP-ribose and nicotinate-adenine dinucleotide phosphate. BST1 expression is enhanced in bone marrow stromal cell lines derived from patients with rheumatoid arthritis. The polyclonal B-cell abnormalities in rheumatoid arthritis may be, at least in part, attributed to BST1 overexpression in the stromal cell population. BST1 also facilitates pre-B-cell growth.

Selectin P (SELP) is a calcium-dependent receptor that binds to sialylated forms of Lewis blood group carbohydrate antigens on neutrophils and monocytes. It is stored in the alpha-granules of platelets and Weibel-Palade bodies of endothelial cells, but redistributes to the plasma membrane during platelet activation and degranulation for mediating the interaction of activated endothelial cells or platelets with leukocytes.

CD200 is a type I membrane glycoprotein containing two extracellular immunoglobulin domains, a transmembrane and a cytoplasmic domain. It is expressed in various cell types, including B cells, a subset of T cells, thymocytes, endothelial cells, and neurons. CD200 plays an important role in immunosuppression and regulation of anti-tumor activity.

Insulin receptor (INSR) is a receptor tyrosine kinase. It is translated as a preproprotein, and proteolytically processed to generate alpha and beta subunits that form a heterotetrameric receptor. INSR is primarily expressed in the liver, adipose tissue and skeletal muscle. Binding of insulin or other ligands to INSR activates the insulin signaling pathway, which regulates glucose uptake and release, as well as the synthesis and storage of carbohydrates, lipids and protein.

Integrin subunit alpha 6 (ITGA6) is a member of the integrin alpha chain family. Integrins are heterodimeric integral membrane proteins composed of an alpha chain and a beta chain that function in cell surface adhesion and signaling. It is translated as a preproprotein, and proteolytically processed to generate light and heavy chains that comprise the alpha 6 subunit. This subunit may associate with a beta 1 or beta 4 subunit to form an integrin that interacts with extracellular matrix proteins including members of the laminin family. The alpha 6 beta 4 integrin may promote tumorigenesis, while the alpha 6 beta 1 integrin may negatively regulate erbB2/HER2 signaling.

Melanotransferrin (MELTF) is a cell-surface glycoprotein of the transferrin superfamily. It is expressed in melanoma cells and in certain fetal tissues. MELTF binds to iron, but the importance of its iron binding activity remains unclear.

Platelet and endothelial cell adhesion molecule 1 (PECAM1) is a cell-surface protein of the immunoglobulin superfamily. It is found on the surface of platelets, monocytes, neutrophils, and some types of T-cells, and makes up a large portion of endothelial cell intercellular junctions. PECAM1 is likely involved in leukocyte migration, angiogenesis, and integrin activation.

Solute carrier family 1 member 5 (SLC1A5) is a sodium-dependent amino acid transporter that has a broad substrate specificity, with a preference for zwitterionic amino acids. It accepts as substrates all neutral amino acids, including glutamine, asparagine, and branched-chain and aromatic amino acids, and excludes methylated, anionic, and cationic amino acids. It is expressed in many tissues, such as fat, prostate, lung, kidney, colon, and placenta. SLC1A5 can also act as a receptor for RD114/type D retrovirus.

### SUMMARY

The invention provides multi-specific binding proteins that bind to the NKG2D receptor and CD16 receptor on natural killer cells, and a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5. Such proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys. Various aspects and embodiments of the invention are described in further detail below.

In certain embodiments, the present invention provides a protein (e.g., a multi-specific binding protein) that binds to, for example, B7-H3 on a cancer cell, and the NKG2D receptor and CD16 receptor on natural killer cells to activate the natural killer cell. The binding protein (e.g., a multi-specific binding protein) is useful in the pharmaceutical compositions and therapeutic methods described herein. Binding of the protein including an antigen-binding site that binds to, for example, B7-H3, and to NKG2D receptor and CD16 receptor on natural killer cell enhances the activity of the natural killer cell toward destruction of a cancer cell. Binding of the protein including an antigen-binding site that binds to, for example, B7-H3 (e.g., a multi-specific binding protein) on a cancer cell brings the cancer cell into proximity to the natural killer cell, which facilitates direct and indirect destruction of the cancer cell by the natural killer cell. Further description of exemplary multi-specific binding proteins is provided below.

The first component of the multi-specific binding proteins of the present disclosure binds to, for example, B7-H3-expressing cells.

The second component of the multi-specific binding proteins of the present disclosure binds to NKG2D receptor-expressing cells, which can include but are not limited to NK cells, γδ T cells and CD8⁺ αβ T cells. Upon NKG2D binding, the multi-specific binding proteins may block natural ligands, such as ULBP6 and MICA, from binding to NKG2D and activating NKG2D receptors.

The third component for the multi-specific binding proteins of the present disclosure binds to cells expressing CD16, an Fc receptor on the surface of leukocytes including natural killer cells, macrophages, neutrophils, eosinophils, mast cells, and follicular dendritic cells.

Some proteins of the present disclosure bind to NKG2D with a K_{D} of 10 nM or weaker affinity.

Accordingly, one aspect of the invention provides a protein that incorporates a first antigen-binding site that binds NKG2D; a second antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5; and an antibody Fc domain, a portion thereof sufficient to bind CD16, or a third antigen-binding site that binds CD16.

The antigen-binding sites may each incorporate an antibody heavy chain variable domain and an antibody light chain variable domain (e.g., arranged as in an antibody, or fused together to from an scFv), or one or more of the antigen-binding sites may be a single domain antibody, such as a V_{H}H antibody like a camelid antibody or a V_{NAR} antibody like those found in cartilaginous fish.

In one aspect, the present invention provides multi-specific binding proteins, which includes a first antigen-binding site that binds NKG2D, a second antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5, an antibody Fc domain, a portion thereof sufficient to bind CD16, or a third antigen-binding site that binds CD16, and an additional antigen-binding site that binds the tumor-associated antigen B7-H3, L1CAM, FLT1, KDR, TNC, TNN, CSPG4, BST1, SELP, CD200, INSR (HHF5), ITGA6, MELTF, PECAM1, or SLC1A5.

The present invention provides a protein in which the first antigen-binding site that binds NKG2D is a single-chain variable fragment (scFv), and the second and/or the additional antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5 is an Fab fragment. The present disclosure also provides a protein in which the first antigen-binding site that binds NKG2D is an scFv, and the second and/or the additional antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5 is an scFv.

The present invention provides a protein in which the first antigen-binding site that binds NKG2D is an Fab fragment, and the second antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5 is an scFv.

The present invention provides a protein in which the first antigen-binding site that binds NKG2D is an scFv, and the second antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5 is an Fab fragment.

In one aspect, a protein of the current invention includes a single-chain variable fragment (scFv) that is linked to an antibody constant domain via a hinge sequence. In some embodiments, the hinge comprises amino acids Ala-Ser. In some other embodiments, the hinge comprises amino acids Ala-Ser or Gly-Ala-Ser. In some embodiments the hinge further comprises amino acids Thr-Lys-Gly. The scFv may include a heavy chain variable domain and a light chain variable domain. In some embodiments, the scFv binds NKG2D or a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5. The hinge sequence provides flexibility of binding to the target antigen.

In some embodiments, a protein of the current invention includes (a) a first antigen-binding site comprising an Fab fragment that binds NKG2D; (b) a second antigen-binding site comprising a single-chain variable fragment (scFv) that binds B7-H3; and (c) an antibody Fc domain or a portion thereof sufficient to bind CD16, or a third antigen-binding site that binds CD16.

In some embodiments of the scFv, the heavy chain variable domain forms a disulfide bridge with the light chain variable domain. For example, a disulfide bridge can be formed between the C44 residue of the heavy chain variable domain and the C100 residue of the light chain variable domain, wherein the amino acid positions are numbered according to the Kabat numbering. In some embodiments, the heavy chain variable domain is linked to the light chain variable domain via a flexible linker, such as a peptide linker comprising the amino acid sequence of GGGGSGGGGSGGGGSGGGGS (“(G4S)₄”) (SEQ ID NO:126). In some embodiments of the scFv, the heavy chain variable domain is positioned at the N terminus of the light chain variable domain. In some embodiments of the scFv, the heavy chain variable domain is positioned at the C terminus of the light chain variable domain.

In one aspect, within the multi-specific binding proteins described above that includes a first antigen-binding site that binds NKG2D, a second antigen-binding site that binds a tumor-associated antigen B7-H3, L1CAM, FLT1, KDR, TNC, TNN, CSPG4, BST1, SELP, CD200, INSR (HHF5), ITGA6, MELTF, PECAM1, or SLC1A5, an antibody Fc domain, a portion thereof sufficient to bind CD16, or a third antigen-binding site that binds CD16, and an additional antigen-binding site that binds the tumor-associated antigen B7-H3, L1CAM, FLT1, KDR, TNC, TNN, CSPG4, BST1, SELP, CD200, INSR (HHF5), ITGA6, MELTF, PECAM1, or SLC1A5; the NKG2D-binding site includes a heavy chain variable domain at least 90% identical to an amino acid sequence selected from: SEQ ID NO:1, SEQ ID NO:41, SEQ ID NO:49, SEQ ID NO:57, SEQ ID NO:59, SEQ ID NO:61, SEQ ID NO:69, SEQ ID NO:77, SEQ ID NO:85, and SEQ ID NO:93.

The first antigen-binding site, which binds to NKG2D, in some embodiments, can incorporate a heavy chain variable domain related to SEQ ID NO:1, such as by having an amino acid sequence at least 90% (e.g., 90%, 91%, 92%, 93%, 94%, 95%, 96%, 97%, 98%, 99%, or 100%) identical to SEQ ID NO:1, and/or incorporating amino acid sequences identical to the CDR1 (SEQ ID NO:105), CDR2 (SEQ ID NO:106), and CDR3 (SEQ ID NO:107) sequences of SEQ ID NO:1. The heavy chain variable domain related to SEQ ID NO:1 can be coupled with a variety of light chain variable domains to form an NKG2D binding site. For example, the first antigen-binding site that incorporates a heavy chain variable domain related to SEQ ID NO:1 can further incorporate a light chain variable domain selected from any one of the sequences related to SEQ ID NOs:2, 4, 6, 8, 10, 12, 14, 16, 18, 20, 22, 24, 26, 28, 30, 32, 34, 36, and 40. For example, the first antigen-binding site incorporates a heavy chain variable domain with amino acid sequences at least 90% (e.g., 90%, 91%, 92%, 93%, 94%, 95%, 96%, 97%, 98%, 99%, or 100%) identical to SEQ ID NO:1 and a light chain variable domain with amino acid sequences at least 90% (e.g., 90%, 91%, 92%, 93%, 94%, 95%, 96%, 97%, 98%, 99%, or 100%) identical to any one of the sequences selected from SEQ ID NOs:2, 4, 6, 8, 10, 12, 14, 16, 18, 20, 22, 24, 26, 28, 30, 32, 34, 36, and 40.

Alternatively, the first antigen-binding site can incorporate a heavy chain variable domain related to SEQ ID NO:41 and a light chain variable domain related to SEQ ID NO:42. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:41, and/or incorporate amino acid sequences identical to the CDR1 (SEQ ID NO:43), CDR2 (SEQ ID NO:44), and CDR3 (SEQ ID NO:45) sequences of SEQ ID NO:41. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:42, and/or incorporate amino acid sequences identical to the CDR1 (SEQ ID NO:46), CDR2 (SEQ ID NO:47), and CDR3 (SEQ ID NO:48) sequences of SEQ ID NO:42.

In other embodiments, the first antigen-binding site can incorporate a heavy chain variable domain related to SEQ ID NO:49 and a light chain variable domain related to SEQ ID NO:50. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:49, and/or incorporate amino acid sequences identical to the CDR1 (SEQ ID NO:51), CDR2 (SEQ ID NO:52), and CDR3 (SEQ ID NO:53) sequences of SEQ ID NO:49. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:50, and/or incorporate amino acid sequences identical to the CDR1 (SEQ ID NO:54), CDR2 (SEQ ID NO:55), and CDR3 (SEQ ID NO:56) sequences of SEQ ID NO:50.

Alternatively, the first antigen-binding site can incorporate a heavy chain variable domain related to SEQ ID NO:57 and a light chain variable domain related to SEQ ID NO:58, such as by having amino acid sequences at least 90% (e.g., 90%, 91%, 92%, 93%, 94%, 95%, 96%, 97%, 98%, 99%, or 100%) identical to SEQ ID NO:57 and at least 90% (e.g., 90%, 91%, 92%, 93%, 94%, 95%, 96%, 97%, 98%, 99%, or 100%) identical to SEQ ID NO:58, respectively.

In another embodiment, the first antigen-binding site can incorporate a heavy chain variable domain related to SEQ ID NO:59 and a light chain variable domain related to SEQ ID NO:60, For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:59, and/or incorporate amino acid sequences identical to the CDR1 (SEQ ID NO:127), CDR2 (SEQ ID NO:128), and CDR3 (SEQ ID NO:129) sequences of SEQ ID NO:59. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:60, and/or incorporate amino acid sequences identical to the CDR1 (SEQ ID NO:130), CDR2 (SEQ ID NO:131), and CDR3 (SEQ ID NO:132) sequences of SEQ ID NO:60.

The first antigen-binding site, which binds to NKG2D, in some embodiments, can incorporate a heavy chain variable domain related to SEQ ID NO:61 and a light chain variable domain related to SEQ ID NO:62. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:61, and/or incorporate amino acid sequences identical to the CDR1 (SEQ ID NO:63 or 341), CDR2 (SEQ ID NO:64), and CDR3 (SEQ ID NO:65 or 342) sequences of SEQ ID NO:61. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:62, and/or incorporate amino acid sequences identical to the CDR1 (SEQ ID NO:66), CDR2 (SEQ ID NO:67), and CDR3 (SEQ ID NO:68) sequences of SEQ ID NO:62. In some embodiments, the first antigen-binding site can incorporate a heavy chain variable domain related to SEQ ID NO:69 and a light chain variable domain related to SEQ ID NO:70. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:69, and/or incorporate amino acid sequences identical to the CDR1 (SEQ ID NO:71 or 343), CDR2 (SEQ ID NO:72), and CDR3 (SEQ ID NO:73 or 344) sequences of SEQ ID NO:69. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:74), CDR2 (SEQ ID NO:75), and CDR3 (SEQ ID NO:76) sequences of SEQ ID NO:70.

In some embodiments, the first antigen-binding site can incorporate a heavy chain variable domain related to SEQ ID NO:77 and a light chain variable domain related to SEQ ID NO:78. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:77, and/or incorporate amino acid sequences identical to the CDR1 (SEQ ID NO:79 or 345), CDR2 (SEQ ID NO:80), and CDR3 (SEQ ID NO:81 or 346) sequences of SEQ ID NO:77. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:78, and/or incorporate amino acid sequences identical to the CDR1 (SEQ ID NO:82), CDR2 (SEQ ID NO:83), and CDR3 (SEQ ID NO:84) sequences of SEQ ID NO:78.

In some embodiments, the first antigen-binding site can incorporate a heavy chain variable domain related to SEQ ID NO:85 and a light chain variable domain related to SEQ ID NO:86. For example, the heavy chain variable domain of the first antigen-binding site can beat least 90% (e.g., 90%, 91%, 92%, 93%, 94%, 95%, 96%, 97%, 98%, 99%, or 100%) identical to SEQ ID NO:85, and/or incorporate amino acid sequences identical to the CDR1 (SEQ ID NO:87 or 347), CDR2 (SEQ ID NO:88), and CDR3 (SEQ ID NO:89 or 348) sequences of SEQ ID NO:85. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:351 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:351, and/or incorporate amino acid sequences identical to the CDR1 (SEQ ID NO:87 or 347), CDR2 (SEQ ID NO:88), and CDR3 (SEQ ID NO:352 or 354) sequences of SEQ ID NO:351. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:353 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:353, and/or incorporate amino acid sequences identical to the CDR1 (SEQ ID NO:87 or 347), CDR2 (SEQ ID NO:88), and CDR3 (SEQ ID NO:355 or 385) sequences of SEQ ID NO:353. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:356 and a light chain variable domain related to SEQ ID NO:86. For example, the heavy chain variable domain of the first antigen-binding site can beat least 90% (e.g., 90%, 91%, 92%, 93%, 94%, 95%, 96%, 97%, 98%, 99%, or 100%) identical to SEQ ID NO:356, and/or incorporate amino acid sequences identical to the CDR1 (SEQ ID NO:87 or 347), CDR2 (SEQ ID NO:88), and CDR3 (SEQ ID NO:357 or 358) sequences of SEQ ID NO:356. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:359 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:359, and/or incorporate amino acid sequences identical to the CDR1 (SEQ ID NO:87 or 347), CDR2 (SEQ ID NO:88), and CDR3 (SEQ ID NO:360 or 361) sequences of SEQ ID NO:359. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:362 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:362, and/or incorporate amino acid sequences identical to the CDR1 (SEQ ID NO:87 or 347), CDR2 (SEQ ID NO:88), and CDR3 (SEQ ID NO:363 or 364) sequences of SEQ ID NO:362. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:365 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:365, and/or incorporate amino acid sequences identical to the CDR1 (SEQ ID NO:87 or 347), CDR2 (SEQ ID NO:88), and CDR3 (SEQ ID NO:366 or 367) sequences of SEQ ID NO:365. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:93 and a light chain variable domain related to SEQ ID NO:94. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:93, and/or incorporate amino acid sequences identical to the CDR1 (SEQ ID NO:95 or 349), CDR2 (SEQ ID NO:96), and CDR3 (SEQ ID NO:97 or 350) sequences of SEQ ID NO:93. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:98), CDR2 (SEQ ID NO:99), and CDR3 (SEQ ID NO:100) sequences of SEQ ID NO:94.

In some embodiments, the first antigen-binding site can incorporate a heavy chain variable domain related to SEQ ID NO:101 and a light chain variable domain related to SEQ ID NO:102, such as by having amino acid sequences at least 90% (e.g., 90%, 91%, 92%, 93%, 94%, 95%, 96%, 97%, 98%, 99%, or 100%) identical to SEQ ID NO:101 and at least 90% (e.g., 90%, 91%, 92%, 93%, 94%, 95%, 96%, 97%, 98%, 99%, or 100%) identical to SEQ ID NO:102, respectively. In some embodiments, the first antigen-binding site can incorporate a heavy chain variable domain related to SEQ ID NO:103 and a light chain variable domain related to SEQ ID NO:104, such as by having amino acid sequences at least 90% (e.g., 90%, 91%, 92%, 93%, 94%, 95%, 96%, 97%, 98%, 99%, or 100%) identical to SEQ ID NO:103 and at least 90% (e.g., 90%, 91%, 92%, 93%, 94%, 95%, 96%, 97%, 98%, 99%, or 100%) identical to SEQ ID NO:104, respectively.

In some embodiments, the second antigen-binding site binding to B7-H3 can incorporate a heavy chain variable domain related to SEQ ID NO:109 or 386 and a light chain variable domain related to SEQ ID NO:113 or 387. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:109 or 386, and/or incorporate amino acid sequences identical to the CDR1 (SEQ ID NO:110), CDR2 (SEQ ID NO:111), and CDR3 (SEQ ID NO:112) sequences of SEQ ID NO:109 or 386. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:113 or 387, and/or incorporate amino acid sequences identical to the CDR1 (SEQ ID NO:114), CDR2 (SEQ ID NO:115), and CDR3 (SEQ ID NO:116) sequences of SEQ ID NO:113 or 387.

Alternatively, the second antigen-binding site binding to B7-H3 can incorporate a heavy chain variable domain related to SEQ ID NO:117 and a light chain variable domain related to SEQ ID NO:121. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:117, and/or incorporate amino acid sequences identical to the CDR1 (SEQ ID NO:118), CDR2 (SEQ ID NO:119), and CDR3 (SEQ ID NO:120) sequences of SEQ ID NO:117. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:121, and/or incorporate amino acid sequences identical to the CDR1 (SEQ ID NO:122), CDR2 (SEQ ID NO:123), and CDR3 (SEQ ID NO:124) sequences of SEQ ID NO:121.

Alternatively, the second antigen-binding site binding to B7-H3 can incorporate a heavy chain variable domain related to SEQ ID NO:369 or 388 and a light chain variable domain related to SEQ ID NO:370 or 389. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:369 or 388, and/or incorporate amino acid sequences identical to the CDR1 (SEQ ID NO:371), CDR2 (SEQ ID NO:372), and CDR3 (SEQ ID NO:373) sequences of SEQ ID NO:369 or 388. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:370 or 389, and/or incorporate amino acid sequences identical to the CDR1 (SEQ ID NO:374), CDR2 (SEQ ID NO:375), and CDR3 (SEQ ID NO:376) sequences of SEQ ID NO:370 or 389.

Alternatively, the second antigen-binding site binding to B7-H3 can incorporate a heavy chain variable domain related to SEQ ID NO:377 or 390 and a light chain variable domain related to SEQ ID NO:378 or 391. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:377 or 390, and/or incorporate amino acid sequences identical to the CDR1 (SEQ ID NO:379), CDR2 (SEQ ID NO:380), and CDR3 (SEQ ID NO:381) sequences of SEQ ID NO:377 or 390. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:378 or 391, and/or incorporate amino acid sequences identical to the CDR1 (SEQ ID NO:382), CDR2 (SEQ ID NO:383), and CDR3 (SEQ ID NO:384) sequences of SEQ ID NO:378 or 391.

In certain embodiments, a protein of the present invention comprising a first antigen-binding site comprising an Fab that binds NKG2D comprises: (1) a heavy chain variable domain comprising CDR1, CDR2, and CDR3 sequences represented by the amino acid sequences of SEQ ID NOs: 347, 88, and 352, respectively; and a light chain variable domain comprising CDR1, CDR2, and CDR3 sequences represented by the amino acid sequences of SEQ ID NOs: 90, 91, and 92, respectively;

(2) a heavy chain variable domain comprising CDR1, CDR2, and CDR3 sequences represented by the amino acid sequences of SEQ ID NOs: 347, 88, and 348, respectively; and a light chain variable domain comprising CDR1, CDR2, and CDR3 sequences represented by the amino acid sequences of SEQ ID NOs: 90, 91, and 92, respectively;

(3) a heavy chain variable domain comprising complementarity-determining region 1 (CDR1), complementarity-determining region 2 (CDR2), and complementarity-determining region 3 (CDR3) sequences represented by the amino acid sequences of SEQ ID NOs: 341, 64, and 342, respectively; and a light chain variable domain comprising CDR1, CDR2, and CDR3 sequences represented by the amino acid sequences of SEQ ID NOs: 66, 67, and 68, respectively;

(4) a heavy chain variable domain comprising CDR1, CDR2, and CDR3 sequences represented by the amino acid sequences of SEQ ID NOs: 343, 72, and 344, respectively; and a light chain variable domain comprising CDR1, CDR2, and CDR3 sequences represented by the amino acid sequences of SEQ ID NOs: 74, 75, and 76, respectively;

(5) a heavy chain variable domain comprising CDR1, CDR2, and CDR3 sequences represented by the amino acid sequences of SEQ ID NOs: 345, 80, and 346, respectively; and a light chain variable domain comprising CDR1, CDR2, and CDR3 sequences represented by the amino acid sequences of SEQ ID NOs: 82, 83, and 84, respectively;

(6) a heavy chain variable domain comprising CDR1, CDR2, and CDR3 sequences represented by the amino acid sequences of SEQ ID NOs: 87, 88, and 89, respectively; and a light chain variable domain comprising CDR1, CDR2, and CDR3 sequences represented by the amino acid sequences of SEQ ID NOs: 90, 91, and 92, respectively;

(7) a heavy chain variable domain comprising CDR1, CDR2, and CDR3 sequences represented by the amino acid sequences of SEQ ID NOs: 349, 96, and 350, respectively; and a light chain variable domain comprising CDR1, CDR2, and CDR3 sequences represented by the amino acid sequences of SEQ ID NOs: 98, 99, and 100, respectively;

(8) a heavy chain variable domain comprising CDR1, CDR2, and CDR3 sequences represented by the amino acid sequences of SEQ ID NOs: 347, 88, and 355, respectively; and a light chain variable domain comprising CDR1, CDR2, and CDR3 sequences represented by the amino acid sequences of SEQ ID NOs: 90, 91, and 92, respectively;

(9) a heavy chain variable domain comprising CDR1, CDR2, and CDR3 sequences represented by the amino acid sequences of SEQ ID NOs: 347, 88, and 358, respectively; and a light chain variable domain comprising CDR1, CDR2, and CDR3 sequences represented by the amino acid sequences of SEQ ID NOs: 90, 91, and 92, respectively;

(10) a heavy chain variable domain comprising CDR1, CDR2, and CDR3 sequences represented by the amino acid sequences of SEQ ID NOs: 347, 88, and 361, respectively; and a light chain variable domain comprising CDR1, CDR2, and CDR3 sequences represented by the amino acid sequences of SEQ ID NOs: 90, 91, and 92, respectively;

(11) a heavy chain variable domain comprising CDR1, CDR2, and CDR3 sequences represented by the amino acid sequences of SEQ ID NOs: 347, 88, and 364, respectively; and a light chain variable domain comprising CDR1, CDR2, and CDR3 sequences represented by the amino acid sequences of SEQ ID NOs: 90, 91, and 92, respectively; or

(12) a heavy chain variable domain comprising CDR1, CDR2, and CDR3 sequences represented by the amino acid sequences of SEQ ID NOs: 347, 88, and 367, respectively; and a light chain variable domain comprising CDR1, CDR2, and CDR3 sequences represented by the amino acid sequences of SEQ ID NOs: 90, 91, and 92, respectively; and a second antigen-binding site comprising a single-chain variable fragment (scFv) that binds B7-H3, comprises a heavy chain variable domain comprising heavy chain CDR1 (CDRH1), heavy chain CDR2 (CDRH2), and heavy chain CDR3 (CDRH3), and a light chain variable domain comprising light chain CDR1 (CDRL1), light chain CDR2 (CDRL2), and light chain CDR3 (CDRL3), wherein the amino acid sequences of CDRH1, CDRH2, CDRH3, CDRL1, CDRL2, and CDRL3 are set forth in SEQ ID NOs: 110, 111, 112, 114, 115, and 116; 118, 119, 120, 122, 123, and 124; 371, 372, 373, 374, 375, and 376; or 379, 380, 381, 382, 383, and 384, respectively.

Certain proteins of the present disclosure include a sequence of SEQ ID NO:329, SEQ ID NO:333, or SEQ ID NO:335.

Certain proteins of the present disclosure include an scFv linked to an antibody Fc domain, wherein the scFv linked to the antibody Fc domain is represented by a sequence selected from SEQ ID NO:330, SEQ ID NO:334 and SEQ ID NO:336.

Certain proteins of the present disclosure include a sequence at least 90% identical to an amino acid sequence of SEQ ID NO:329, SEQ ID NO:333, or SEQ ID NO:335.

Certain proteins of the present disclosure include a sequence at least 95% identical to an amino acid sequence of SEQ ID NO:329, SEQ ID NO:333, or SEQ ID NO:335.

Certain proteins of the present disclosure include a sequence at least 99% identical to an amino acid sequence of SEQ ID NO:329, SEQ ID NO:333, or SEQ ID NO:335.

Certain proteins of the present disclosure include a sequence at least 90% identical to an amino acid sequence selected from SEQ ID NO:330, SEQ ID NO:334 and SEQ ID NO:336.

Certain proteins of the present disclosure include a sequence at least 95% identical to an amino acid sequence selected from SEQ ID NO:330, SEQ ID NO:334 and SEQ ID NO:336.

Certain proteins of the present disclosure include a sequence at least 99% identical to an amino acid sequence selected from SEQ ID NO:330, SEQ ID NO:334 and SEQ ID NO:336.

Certain proteins of the present disclosure include a B7-H3-binding scFv (SEQ ID NO:329, SEQ ID NO:333, or SEQ ID NO:335) linked to an Fc domain via a hinge comprising Ala-Ser (scFv-Fc represented by SEQ ID NO:330, SEQ ID NO:334 and SEQ ID NO:336); and an NKG2D-binding Fab fragment including a heavy chain portion comprising a heavy chain variable domain comprising SEQ ID NO:351 and a CH1 domain, and a light chain portion comprising a light chain variable domain (SEQ ID NO:86) and a light chain constant domain, where the heavy chain variable domain is connected to the CH1 domain, and the CH1 domain is connected to the Fc domain (heavy chain portion represented as V_{H}-CH1-Fc, amino acid sequence set forth in SEQ ID NO:331).

In some embodiments, the second antigen-binding site binding to L1CAM can incorporate a heavy chain variable domain related to SEQ ID NO:133 and a light chain variable domain related to SEQ ID NO:137. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:133, and/or incorporate amino acid sequences identical to the CDR1 (SEQ ID NO:134), CDR2 (SEQ ID NO:135), and CDR3 (SEQ ID NO:136) sequences of SEQ ID NO:133. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:137, and/or incorporate amino acid sequences identical to the CDR1 (SEQ ID NO:138), CDR2 (SEQ ID NO:139), and CDR3 (SEQ ID NO:140) sequences of SEQ ID NO:137.

Alternatively, the second antigen-binding site binding to L1CAM can incorporate a heavy chain variable domain related to SEQ ID NO:141 and a light chain variable domain related to SEQ ID NO:145. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:141, and/or incorporate amino acid sequences identical to the CDR1 (SEQ ID NO:142), CDR2 (SEQ ID NO:143), and CDR3 (SEQ ID NO:144) sequences of SEQ ID NO:141. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:145, and/or incorporate amino acid sequences identical to the CDR1 (SEQ ID NO:146), CDR2 (SEQ ID NO:147), and CDR3 (SEQ ID NO:148) sequences of SEQ ID NO:145.

In some embodiments, the second antigen-binding site binding to FLT1 can incorporate a heavy chain variable domain related to SEQ ID NO:150 and a light chain variable domain related to SEQ ID NO:154. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:150, and/or incorporate amino acid sequences identical to the CDR1 (SEQ ID NO:151), CDR2 (SEQ ID NO:152), and CDR3 (SEQ ID NO:153) sequences of SEQ ID NO:150. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:154, and/or incorporate amino acid sequences identical to the CDR1 (SEQ ID NO:155), CDR2 (SEQ ID NO:156), and CDR3 (SEQ ID NO:157) sequences of SEQ ID NO:154.

Alternatively, the second antigen-binding site binding to FLT1 can incorporate a heavy chain variable domain related to SEQ ID NO:158 and a light chain variable domain related to SEQ ID NO:162. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:158, and/or incorporate amino acid sequences identical to the CDR1 (SEQ ID NO:159), CDR2 (SEQ ID NO:160), and CDR3 (SEQ ID NO:161) sequences of SEQ ID NO:158. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:162, and/or incorporate amino acid sequences identical to the CDR1 (SEQ ID NO:163), CDR2 (SEQ ID NO:164), and CDR3 (SEQ ID NO:165) sequences of SEQ ID NO:162.

Alternatively, the second antigen-binding site binding to KDR can incorporate a heavy chain variable domain related to SEQ ID NO:166 and a light chain variable domain related to SEQ ID NO:170. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:166, and/or incorporate amino acid sequences identical to the CDR1 (SEQ ID NO:167), CDR2 (SEQ ID NO:168), and CDR3 (SEQ ID NO:169) sequences of SEQ ID NO:166. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:170, and/or incorporate amino acid sequences identical to the CDR1 (SEQ ID NO:171), CDR2 (SEQ ID NO:172), and CDR3 (SEQ ID NO:173) sequences of SEQ ID NO:170.

Alternatively, the second antigen-binding site binding to KDR can incorporate a heavy chain variable domain related to SEQ ID NO:174 and a light chain variable domain related to SEQ ID NO:178. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:174, and/or incorporate amino acid sequences identical to the CDR1 (SEQ ID NO:175), CDR2 (SEQ ID NO:176), and CDR3 (SEQ ID NO:177) sequences of SEQ ID NO:174. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:178, and/or incorporate amino acid sequences identical to the CDR1 (SEQ ID NO:179), CDR2 (SEQ ID NO:180), and CDR3 (SEQ ID NO:181) sequences of SEQ ID NO:178.

Alternatively, the second antigen-binding site binding to TNC can incorporate a heavy chain variable domain related to SEQ ID NO:182 and a light chain variable domain related to SEQ ID NO:186. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:182, and/or incorporate amino acid sequences identical to the CDR1 (SEQ ID NO:183), CDR2 (SEQ ID NO:184), and CDR3 (SEQ ID NO:185) sequences of SEQ ID NO:182. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:186, and/or incorporate amino acid sequences identical to the CDR1 (SEQ ID NO:187), CDR2 (SEQ ID NO:188), and CDR3 (SEQ ID NO:189) sequences of SEQ ID NO:186.

Alternatively, the second antigen-binding site binding to TNC can incorporate a heavy chain variable domain related to SEQ ID NO:190 and a light chain variable domain related to SEQ ID NO:194. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:190, and/or incorporate amino acid sequences identical to the CDR1 (SEQ ID NO:191), CDR2 (SEQ ID NO:192), and CDR3 (SEQ ID NO:193) sequences of SEQ ID NO:190. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:194, and/or incorporate amino acid sequences identical to the CDR1 (SEQ ID NO:195), CDR2 (SEQ ID NO:196), and CDR3 (SEQ ID NO:197) sequences of SEQ ID NO:194.

Alternatively, the second antigen-binding site binding to CSPG4 can incorporate a heavy chain variable domain related to SEQ ID NO:198 and a light chain variable domain related to SEQ ID NO:202. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:198, and/or incorporate amino acid sequences identical to the CDR1 (SEQ ID NO:199), CDR2 (SEQ ID NO:200), and CDR3 (SEQ ID NO:201) sequences of SEQ ID NO:198. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:202, and/or incorporate amino acid sequences identical to the CDR1 (SEQ ID NO:203), CDR2 (SEQ ID NO:204), and CDR3 (SEQ ID NO:205) sequences of SEQ ID NO:202.

Alternatively, the second antigen-binding site binding to CSPG4 can incorporate a heavy chain variable domain related to SEQ ID NO:206 and a light chain variable domain related to SEQ ID NO:210. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:206, and/or incorporate amino acid sequences identical to the CDR1 (SEQ ID NO:207), CDR2 (SEQ ID NO:208), and CDR3 (SEQ ID NO:209) sequences of SEQ ID NO:206. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:210, and/or incorporate amino acid sequences identical to the CDR1 (SEQ ID NO:211), CDR2 (SEQ ID NO:212), and CDR3 (SEQ ID NO:213) sequences of SEQ ID NO:210.

Alternatively, the second antigen-binding site binding to BST1 can incorporate a heavy chain variable domain related to SEQ ID NO:214 and a light chain variable domain related to SEQ ID NO:218. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:214, and/or incorporate amino acid sequences identical to the CDR1 (SEQ ID NO:215), CDR2 (SEQ ID NO:216), and CDR3 (SEQ ID NO:217) sequences of SEQ ID NO:214. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:218, and/or incorporate amino acid sequences identical to the CDR1 (SEQ ID NO:219), CDR2 (SEQ ID NO:220), and CDR3 (SEQ ID NO:221) sequences of SEQ ID NO:218.

Alternatively, the second antigen-binding site binding to BST1 can incorporate a heavy chain variable domain related to SEQ ID NO:222 and a light chain variable domain related to SEQ ID NO:226. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:222, and/or incorporate amino acid sequences identical to the CDR1 (SEQ ID NO:223), CDR2 (SEQ ID NO:224), and CDR3 (SEQ ID NO:225) sequences of SEQ ID NO:222. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:226, and/or incorporate amino acid sequences identical to the CDR1 (SEQ ID NO:227), CDR2 (SEQ ID NO:228), and CDR3 (SEQ ID NO:229) sequences of SEQ ID NO:226.

Alternatively, the second antigen-binding site binding to SELP can incorporate a heavy chain variable domain related to SEQ ID NO:230 and a light chain variable domain related to SEQ ID NO:234. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:230, and/or incorporate amino acid sequences identical to the CDR1 (SEQ ID NO:231), CDR2 (SEQ ID NO:232), and CDR3 (SEQ ID NO:233) sequences of SEQ ID NO:230. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:234, and/or incorporate amino acid sequences identical to the CDR1 (SEQ ID NO:235), CDR2 (SEQ ID NO:236), and CDR3 (SEQ ID NO:237) sequences of SEQ ID NO:234.

Alternatively, the second antigen-binding site binding to SELP can incorporate a heavy chain variable domain related to SEQ ID NO:238 and a light chain variable domain related to SEQ ID NO:242. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:238, and/or incorporate amino acid sequences identical to the CDR1 (SEQ ID NO:239), CDR2 (SEQ ID NO:240), and CDR3 (SEQ ID NO:241) sequences of SEQ ID NO:238. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:242, and/or incorporate amino acid sequences identical to the CDR1 (SEQ ID NO:243), CDR2 (SEQ ID NO:244), and CDR3 (SEQ ID NO:245) sequences of SEQ ID NO:242.

Alternatively, the second antigen-binding site binding to CD200 can incorporate a heavy chain variable domain related to SEQ ID NO:246 and a light chain variable domain related to SEQ ID NO:250. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:246, and/or incorporate amino acid sequences identical to the CDR1 (SEQ ID NO:247), CDR2 (SEQ ID NO:248), and CDR3 (SEQ ID NO:249) sequences of SEQ ID NO:246. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:250, and/or incorporate amino acid sequences identical to the CDR1 (SEQ ID NO:251), CDR2 (SEQ ID NO:252), and CDR3 (SEQ ID NO:253) sequences of SEQ ID NO:250.

Alternatively, the second antigen-binding site binding to INSR (HHF5) can incorporate a heavy chain variable domain related to SEQ ID NO:254 and a light chain variable domain related to SEQ ID NO:258. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:254, and/or incorporate amino acid sequences identical to the CDR1 (SEQ ID NO:255), CDR2 (SEQ ID NO:256), and CDR3 (SEQ ID NO:257) sequences of SEQ ID NO:254. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:258, and/or incorporate amino acid sequences identical to the CDR1 (SEQ ID NO:259), CDR2 (SEQ ID NO:260), and CDR3 (SEQ ID NO:261) sequences of SEQ ID NO:258.

Alternatively, the second antigen-binding site binding to INSR (HHF5) can incorporate a heavy chain variable domain related to SEQ ID NO:262 and a light chain variable domain related to SEQ ID NO:266. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:262, and/or incorporate amino acid sequences identical to the CDR1 (SEQ ID NO:263), CDR2 (SEQ ID NO:264), and CDR3 (SEQ ID NO:265) sequences of SEQ ID NO:262. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:266, and/or incorporate amino acid sequences identical to the CDR1 (SEQ ID NO:267), CDR2 (SEQ ID NO:268), and CDR3 (SEQ ID NO:269) sequences of SEQ ID NO:266.

Alternatively, the second antigen-binding site binding to ITGA6 can incorporate a heavy chain variable domain related to SEQ ID NO:270 and a light chain variable domain related to SEQ ID NO:274. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:270, and/or incorporate amino acid sequences identical to the CDR1 (SEQ ID NO:271), CDR2 (SEQ ID NO:272), and CDR3 (SEQ ID NO:273) sequences of SEQ ID NO:270. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:274, and/or incorporate amino acid sequences identical to the CDR1 (SEQ ID NO:275), CDR2 (SEQ ID NO:276), and CDR3 (SEQ ID NO:277) sequences of SEQ ID NO:274.

Alternatively, the second antigen-binding site binding to ITGA6 can incorporate a heavy chain variable domain sequence comprising a CDR1 sequence identical to the amino acid sequence of SEQ ID NO:278, a CDR2 sequence identical to the amino acid sequence of SEQ ID NO:279, and a CDR3 sequence identical to the amino acid sequence of SEQ ID NO:280; and a light chain variable domain sequence comprising a CDR1 sequence identical to the amino acid sequence of SEQ ID NO:281, a CDR2 sequence identical to the amino acid sequence of SEQ ID NO:282, and a CDR3 sequence identical to the amino acid sequence of SEQ ID NO:283.

Alternatively, the second antigen-binding site binding MELTF can incorporate a heavy chain variable domain related to SEQ ID NO:284 and a light chain variable domain related to SEQ ID NO:288. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:284, and/or incorporate amino acid sequences identical to the CDR1 (SEQ ID NO:285), CDR2 (SEQ ID NO:286), and CDR3 (SEQ ID NO:287) sequences of SEQ ID NO:284. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:288, and/or incorporate amino acid sequences identical to the CDR1 (SEQ ID NO:289), CDR2 (SEQ ID NO:290), and CDR3 (SEQ ID NO:291) sequences of SEQ ID NO:288.

Alternatively, the second antigen-binding site binding to MELTF can incorporate a heavy chain variable domain related to SEQ ID NO:292 and a light chain variable domain related to SEQ ID NO:296. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:292, and/or incorporate amino acid sequences identical to the CDR1 (SEQ ID NO:293), CDR2 (SEQ ID NO:294), and CDR3 (SEQ ID NO:295) sequences of SEQ ID NO:292. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:296, and/or incorporate amino acid sequences identical to the CDR1 (SEQ ID NO:297), CDR2 (SEQ ID NO:298), and CDR3 (SEQ ID NO:299) sequences of SEQ ID NO:296.

Alternatively, the second antigen-binding site binding to SLC1A5 can incorporate a heavy chain variable domain related to SEQ ID NO:300 and a light chain variable domain related to SEQ ID NO:304. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:300, and/or incorporate amino acid sequences identical to the CDR1 (SEQ ID NO:301), CDR2 (SEQ ID NO:302), and CDR3 (SEQ ID NO:303) sequences of SEQ ID NO:300. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:304, and/or incorporate amino acid sequences identical to the CDR1 (SEQ ID NO:305), CDR2 (SEQ ID NO:306), and CDR3 (SEQ ID NO:307) sequences of SEQ ID NO:304.

Alternatively, the second antigen-binding site binding to SLC1A5 can incorporate a heavy chain variable domain related to SEQ ID NO:308 and a light chain variable domain related to SEQ ID NO:312. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:308, and/or incorporate amino acid sequences identical to the CDR1 (SEQ ID NO:309), CDR2 (SEQ ID NO:310), and CDR3 (SEQ ID NO:311) sequences of SEQ ID NO:308. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:312, and/or incorporate amino acid sequences identical to the CDR1 (SEQ ID NO:313), CDR2 (SEQ ID NO:314), and CDR3 (SEQ ID NO:315) sequences of SEQ ID NO:312.

In some embodiments, the second and/or additional antigen-binding site incorporate(s) a light chain variable domain having an amino acid sequence identical to the amino acid sequence of the light chain variable domain present in the first antigen-binding site.

In some embodiments, the multi-specific binding proteins incorporate a portion of an antibody Fc domain sufficient to bind CD16, wherein the antibody Fc domain comprises hinge and CH2 domains, and/or amino acid sequences at least 90% identical to amino acid sequence 234-332 of a human IgG antibody. Mutations can be introduced into the antibody constant domain to enable heterdimerization with another antibody constant domain. For example, if the antibody constant domain is derived from the constant domain of a human IgG1, the antibody constant domain can include an amino acid sequence at least 90% identical to amino acids 234-332 of a human IgG1 antibody, and differs at one or more positions selected from the group consisting of Q347, Y349, L351, Q352, S354, E356, E357, K360, Q362, S364, T366, L368, K370, N390, K392, T394, D399, S400, D401, F405, Y407, K409, T411, and K439, wherein the amino acid positions are numbered according to the EU numbering.

In some embodiments, the antibody constant domain can comprise an amino acid sequence at least 90% identical to amino acids 234-332 of a human IgG1 antibody, and differs by one or more substitutions selected from the group consisting of Q347E, Q347R, Y349S, Y349K, Y349T, Y349D, Y349E, Y349C, L351K, L351D, L351Y, Q352E, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, wherein the amino acid positions are numbered according to the EU numbering.

Formulations containing any one of the proteins described herein; cells containing one or more nucleic acids expressing the proteins, and methods of enhancing tumor cell death using the proteins are also provided.

Another aspect of the invention provides a method of treating cancer in a patient. The method comprises administering to a patient in need thereof a therapeutically effective amount of the multi-specific binding proteins described herein. Exemplary cancers to be treated using the multi-specific binding proteins include bladder cancer, breast cancer, cervical cancer, glioblastoma, head and neck cancer, lung cancer, liver cancer, melanoma, ovarian cancer, pancreatic cancer, prostate cancer, sarcoma, renal cancer, colorectal cancer, gastric cancer, neuroblastoma, squamous cell carcinoma, and acute myeloid leukemia (AML).

### BRIEF DESCRIPTION OF THE DRAWINGS

FIG. 1 illustrate an exemplary format of a multi-specific binding protein, e.g., a trispecific binding protein (TriNKET). Each arm can represent either the NKG2D-binding domain, or the B7-H3 binding domain. In some embodiments, the NKG2D- and the B7-H3-binding domains can share a common light chain.

FIGS. 2A to 2E illustrate five exemplary formats of a multi-specific binding protein, e.g., a trispecific binding protein (TriNKET). As shown in FIG. 2A, an antibody that contains an NKG2D-targeting scFv, a B7-H3-targeting Fab fragment, and a heterodimerized antibody constant region is referred herein as the F3-TriNKET. As shown in FIG. 2B, an antibody that contains a B7-H3-targeting scFv, a NKG2D-targeting Fab fragment, and a heterodimerized antibody constant region/domain that binds CD16 is referred herein as the F3′-TriNKET. As shown in FIG. 2C, both the NKG2D-binding domain and B7-H3-binding domain can take the scFv format. FIGS. 2D to 2E are illustrations of an antibody with three antigen-binding sites, including two antigen-binding sites that bind B7-H3, and an NKG2D-binding site fused to the heterodimerized antibody constant region. These antibody formats are referred herein as F4-TriNKET. FIG. 2D illustrates that the two B7-H3-binding sites are in the Fab format, and the NKG2D binding site in the scFv format, referred herein as the F4-TriNKET. FIG. 2E illustrates that the B7-H3-binding sites in the scFv format, and the NKG2D binding site in the scFv format. In certain exemplary multispecific binding proteins, heterodimerization mutations on the antibody constant region include K360E and K409W on one constant domain (“CD domain”); and Q347R, D399V and F405T on the opposite constant domain (shown as a triangular lock-and-key shape in the CD domains). The bold bar between the heavy and the light chain variable domains of the Fab fragments represents a disulfide bond.

FIG. 3 are line graphs demonstrating the binding affinity of NKG2D-binding domains (listed as clones) to human recombinant NKG2D in an ELISA assay.

FIG. 4 are line graphs demonstrating the binding affinity of NKG2D-binding domains (listed as clones) to cynomolgus recombinant NKG2D in an ELISA assay.

FIG. 5 are line graphs demonstrating the binding affinity of NKG2D-binding domains (listed as clones) to mouse recombinant NKG2D in an ELISA assay.

FIG. 6 are bar graphs demonstrating the binding of NKG2D-binding domains (listed as clones) to EL4 cells expressing human NKG2D by flow cytometry showing mean fluorescence intensity (MFI) fold over background (FOB).

FIG. 7 are bar graphs demonstrating the binding of NKG2D-binding domains (listed as clones) to EL4 cells expressing mouse NKG2D by flow cytometry showing mean fluorescence intensity (MFI) fold over background (FOB).

FIG. 8 are line graphs demonstrating specific binding affinity of NKG2D-binding domains (listed as clones) to recombinant human NKG2D-Fc by competing with natural ligand ULBP-6.

FIG. 9 are line graphs demonstrating specific binding affinity of NKG2D-binding domains (listed as clones) to recombinant human NKG2D-Fc by competing with natural ligand MICA.

FIG. 10 are line graphs demonstrating specific binding affinity of NKG2D-binding domains (listed as clones) to recombinant mouse NKG2D-Fc by competing with natural ligand Rae-1 delta.

FIG. 11 are bar graphs showing activation of human NKG2D by NKG2D-binding domains (listed as clones) by quantifying the percentage of TNF-α positive cells, which express human NKG2D-CD3 zeta fusion proteins.

FIG. 12 are bar graphs showing activation of mouse NKG2D by NKG2D-binding domains (listed as clones) by quantifying the percentage of TNF-α positive cells, which express mouse NKG2D-CD3 zeta fusion proteins.

FIG. 13 are bar graphs showing activation of human NK cells by NKG2D-binding domains (listed as clones).

FIG. 14 are bar graphs showing activation of human NK cells by NKG2D-binding domains (listed as clones).

FIG. 15 are bar graphs showing activation of mouse NK cells by NKG2D-binding domains (listed as clones).

FIG. 16 are bar graphs showing activation of mouse NK cells by NKG2D-binding domains (listed as clones).

FIG. 17 are bar graphs showing the cytotoxic effect of NKG2D-binding domains (listed as clones) on tumor cells.

FIG. 18 are bar graphs showing the melting temperature of NKG2D-binding domains (listed as clones) measured by differential scanning fluorimetry.

FIGS. 19A to 19C are bar graphs of synergistic activation of NK cells using CD16 and NKG2D-binding. FIG. 19A demonstrates levels of CD107a; FIG. 19B demonstrates levels of IFN-γ; FIG. 19C demonstrates levels of CD107a and IFN-γ. Graphs indicate the mean (n=2)±SD. Data are representative of five independent experiments using five different healthy donors.

FIG. 20 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 21 is a representation of a TriNKET in the KiH Common Light Chain form, which involves the knobs-into-holes (KIHs) technology. KiH is a heterodimer containing 2 Fab fragments binding to target 1 and 2, and an Fc stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 22 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of Fab fragment targeting antigen 1. Construct contains normal Fc.

FIG. 23 is a representation of a TriNKET in the Orthogonal Fab fragment interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc.

FIG. 24 is a representation of a TriNKET in the 2-in-1 Ig format.

FIG. 25 is a representation of a TriNKET in the ES form, which is a heterodimeric construct containing two different Fab fragments binding to target 1 and target 2 fused to the Fc. Heterodimerization is ensured by electrostatic steering mutations in the Fc.

FIG. 26 is a representation of a TriNKET in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc stabilized by heterodimerization mutations.

FIG. 27 is a representation of a TriNKET in the SEED Body form, which is a heterodimer containing 2 Fab fragments binding to target 1 and 2, and an Fc stabilized by heterodimerization mutations.

FIG. 28 is a representation of a TriNKET in the LuZ-Y form, in which a leucine zipper is used to induce heterodimerization of two different HCs. The LuZ-Y form is a heterodimer containing two different scFabs binding to target 1 and 2, fused to Fc. Heterodimerization is ensured through leucine zipper motifs fused to C-terminus of Fc.

FIG. 29 is a representation of a TriNKET in the Cov-X-Body form.

FIGS. 30A to 30B are representations of TriNKETs in the κλ-Body forms, which are heterodimeric constructs with two different Fab fragments fused to Fc stabilized by heterodimerization mutations: one Fab fragment targeting antigen 1 contains kappa LC, and the second Fab targeting antigen 2 contains lambda LC. FIG. 30A is an exemplary representation of one form of a κλ-Body; FIG. 30B is an exemplary representation of another κλ-Body.

FIG. 31 is an Oasc-Fab heterodimeric construct that includes Fab fragment binding to target 1 and scFab binding to target 2, both of which are fused to the Fc domain. Heterodimerization is ensured by mutations in the Fc domain.

FIG. 32 is a DuetMab, which is a heterodimeric construct containing two different Fab fragments binding to antigens 1 and 2, and an Fc that is stabilized by heterodimerization mutations. Fab fragments 1 and 2 contain differential S-S bridges that ensure correct light chain and heavy chain pairing.

FIG. 33 is a CrossmAb, which is a heterodimeric construct with two different Fab fragments binding to targets 1 and 2, and an Fc stabilized by heterodimerization mutations. CL and CH1 domains, and VH and VL domains are switched, e.g., CH1 is fused in-line with VL, while CL is fused in-line with VH.

FIG. 34 is a Fit-Ig, which is a homodimeric construct where Fab fragment binding to antigen 2 is fused to the N-terminus of HC of Fab fragment that binds to antigen 1. The construct contains wild-type Fc.

FIG. 35 are line graphs demonstrating binding of B7-H3-targeted TriNKETs and their parental monoclonal antibodies to B7-H3-expressing human cancer cell lines (A) 786-O, (B) BT-474 and (C) HCC1954.

FIG. 36 are bar graphs demonstrating that TriNKETs enhance NK cell-mediated lysis of B7-H3-expressing cancer cells better than parental mAbs as measured by DELFIA cytotoxicity assay to measure percent specific lysis.

FIGS. 37A to 37B are line graphs demonstrating KHYG-1 CD16V cell killing of BT-474 (FIG. 37A) and HCC1954 (FIG. 37B) target cells mediated by B7-H3 TriNKETs and parental monoclonal antibodies as measured by DELFIA cytotoxicity assay to measure percent specific lysis and indicating that TriNKETs are more potent (lower EC50) and reach higher maximum lysis than their parental mAbs.

FIGS. 38A to 38B are line graphs demonstrating activation of human NK cells with BT-474 (FIG. 38A) and 786-O (FIG. 38B) cells in the presence of B7-H3 TriNKETs or parental monoclonal antibodies. The percentage of IFN and CD107a double-positive NK cells were higher in co-cultures treated with 10 μg/ml of B7-H3 TriNKETs compared to their respective parental mAbs, indicating that TriNKETs stimulate NK cells better than their parental mAbs.

### DETAILED DESCRIPTION

The invention provides multi-specific binding proteins that bind the NKG2D receptor and CD16 receptor on natural killer cells, and the tumor-associated antigen B7-H3. In some embodiments, the multi-specific proteins further include an additional antigen-binding site that binds B7-H3 or another tumor-associated antigen. The invention also provides pharmaceutical compositions comprising such multi-specific binding proteins, and therapeutic methods using such multi-specific proteins and pharmaceutical compositions, for purposes such as treating cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor associated antigen” as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW4⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

In one aspect the invention provides multi-specific binding proteins that bind to the NKG2D receptor and CD16 receptor on natural killer cells, and the tumor-associated antigen B7-H3. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of tumor cells expressing B7-H3. Binding of the multi-specific binding proteins to B7-H3-expressing cells brings the cancer cells into proximity with the natural killer cell, which facilitates direct and indirect destruction of the cancer cells by the natural killer cell. Further description of some exemplary multi-specific binding proteins is provided below.

In another aspect the invention provides multi-specific binding proteins that bind to the NKG2D receptor and CD16 receptor on natural killer cells, and the tumor-associated antigen L1CAM. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of tumor cells expressing L1CAM. Binding of the multi-specific binding proteins to L1CAM-expressing cells brings the cancer cells into proximity with the natural killer cell, which facilitates direct and indirect destruction of the cancer cells by the natural killer cell.

In yet another aspect the invention provides multi-specific binding proteins that bind to the NKG2D receptor and CD16 receptor on natural killer cells, and a tumor-associated antigen selected from the group consisting of FLT1, KDR, TNC, TNN, CSPG4, BST1, SELP, CD200, INSR (HHF5), ITGA6, MELTF, PECAM1, or SLC1A5. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of tumor cells expressing FLT1, KDR, TNC, TNN, CSPG4, BST1, SELP, CD200, INSR (HHF5), ITGA6, MELTF, PECAM1, or SLC1A5. Binding of the multi-specific binding proteins to FLT1, KDR, TNC, TNN, CSPG4, BST1, SELP, CD200, INSR (HHF5), ITGA6, MELTF, PECAM1, or SLC1A5-expressing cells brings the cancer cells into proximity with the natural killer cell, which facilitates direct and indirect destruction of the cancer cells by the natural killer cell.

The first component of the multi-specific binding proteins binds to NKG2D receptor-expressing cells, which can include but are not limited to NK cells, γδ T cells and CD8⁺ αβ T cells. Upon NKG2D binding, the multi-specific binding proteins may block natural ligands, such as ULBP6 and MICA, from binding to NKG2D and activating NK cells.

In some embodiments, the second component of the multi-specific binding proteins binds B7-H3. B7-H3-expressing cells may be found in bladder cancer, breast cancer, cervical cancer, glioblastoma, head and neck cancer, lung cancer, liver cancer, melanoma, ovarian cancer, pancreatic cancer, prostate cancer, sarcoma, renal cancer, colorectal cancer, gastric cancer, neuroblastoma, squamous cell carcinoma, and acute myeloid leukemia (AML).

In some embodiments, the second component of the multi-specific binding proteins binds L1CAM. L1CAM-expressing cells may be found in bladder cancer, renal cancer, breast cancer, cervical cancer, sarcoma, lung cancer, head and neck cancer, glioblastoma, neuroblastoma, melanoma, ovarian cancer, endometrial cancer, esophageal cancer, gastric cancer, gastrointestinal stromal tumor (GIST), cholangiocarcinoma, colorectal cancer, pancreatic cancer, and prostate cancer.

In some embodiments, the second component of the multi-specific binding proteins binds FLT1, KDR, TNC, TNN, CSPG4, BST1, SELP, CD200, INSR (HHF5), ITGA6, MELTF, PECAM1, or SLC1A5. FLT1, KDR, TNC, TNN, CSPG4, BST1, SELP, CD200, INSR (HHF5), ITGA6, MELTF, PECAM1, or SLC1A5-expressing cells may be found in leukemia, for example, acute myeloid leukemia and T-cell leukemia.

The third component of the multi-specific binding proteins binds to cells expressing CD16, an Fc receptor on the surface of leukocytes including natural killer cells, macrophages, neutrophils, eosinophils, mast cells, and follicular dendritic cells.

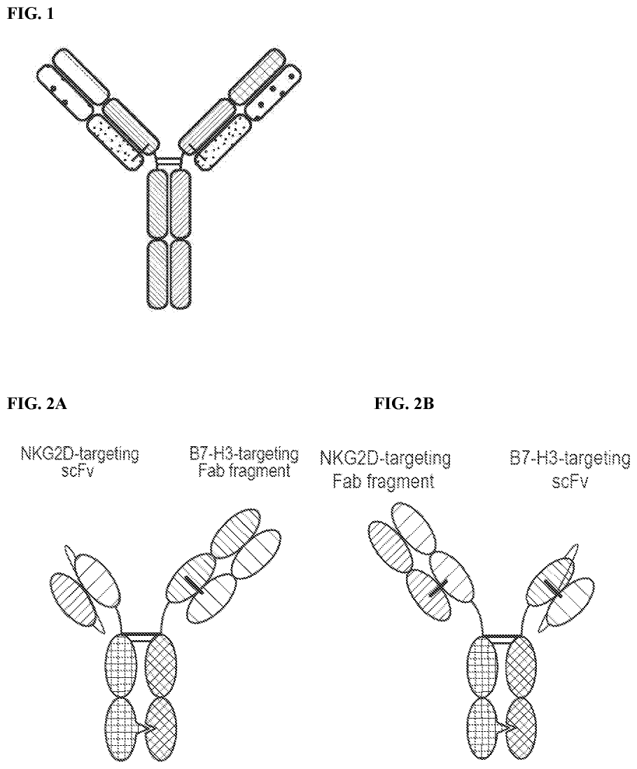

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and optionally a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and optionally a second light chain constant domain. The second immunoglobulin light chain, together with the second immunoglobulin heavy chain, forms an antigen-binding site that binds B7-H3, L1CAM, FLT1, KDR, TNC, TNN, CSPG4, BST1, SELP, CD200, INSR (HHF5), ITGA6, MELTF, PECAM1, or SLC1A5. The first Fc domain and second Fc domain together are able to bind to CD16 (FIG. 1). In some embodiments, the first immunoglobulin light chain is identical to the second immunoglobulin light chain.

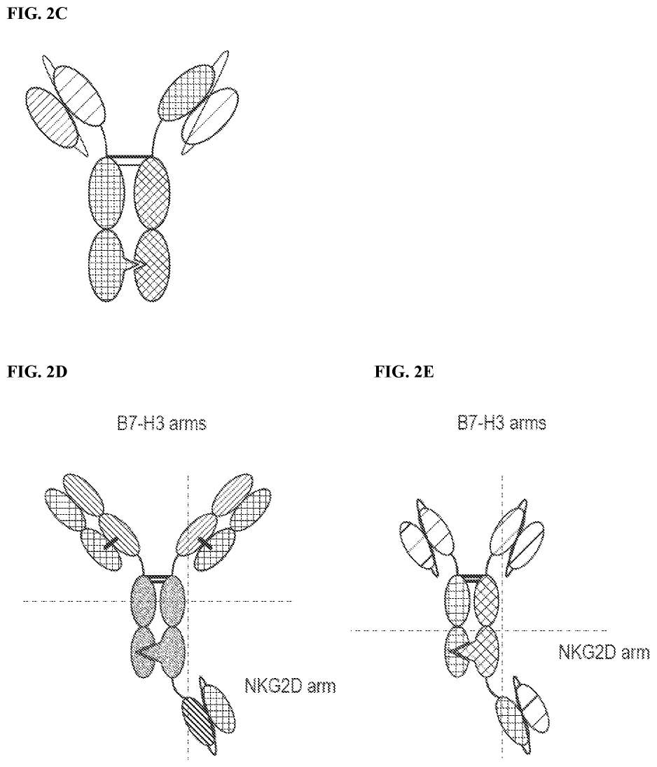

Another exemplary format involves a heterodimeric, multi-specific antibody including a first immunoglobulin heavy chain, a second immunoglobulin heavy chain and an immunoglobulin light chain (FIGS. 2A and 2B). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind the B7-H3, L1CAM, FLT1, KDR, TNC, TNN, CSPG4, BST1, SELP, CD200, INSR (HHF5), ITGA6, MELTF, PECAM1, or SLC1A5 antigen. The second immunoglobulin heavy chain includes a second Fc (hinge-CH2-CH3) domain, a second heavy chain variable domain and a CH1 heavy chain domain. The immunoglobulin light chain includes a light chain variable domain and a light chain constant domain. The second immunoglobulin heavy chain pairs with the immunoglobulin light chain and binds to NKG2D or binds the tumor-associated antigen B7-H3, L1CAM, FLT1, KDR, TNC, TNN, CSPG4, BST1, SELP, CD200, INSR (HHF5), ITGA6, MELTF, PECAM1, or SLC1A5. The first Fc domain and the second Fc domain together are able to bind to CD16 (FIGS. 2A and 2B).

Another exemplary format involves a heterodimeric, multi-specific antibody including a first immunoglobulin heavy chain, and a second immunoglobulin heavy chain (FIG. 2C). The first immunoglobulin heavy chain includes a first Fc (hinge-CH2-CH3) domain fused via either a linker or an antibody hinge to a single-chain variable fragment (scFv) composed of a heavy chain variable domain and light chain variable domain which pair and bind NKG2D, or bind the B7-H3, L1CAM, FLT1, KDR, TNC, TNN, CSPG4, BST1, SELP, CD200, INSR (HHF5), ITGA6, MELTF, PECAM1, or SLC1A5 antigen. The second immunoglobulin heavy chain includes a second Fc (hinge-CH2-CH3) domain fused via either a linker or an antibody hinge to a single-chain variable fragment (scFv) composed of a heavy chain variable domain and light chain variable domain which pair and bind NKG2D, or bind the B7-H3, L1CAM, FLT1, KDR, TNC, TNN, CSPG4, BST1, SELP, CD200, INSR (HHF5), ITGA6, MELTF, PECAM1, or SLC1A5. The first Fc domain and the second Fc domain together are able to bind to CD16 (FIG. 2C).

In some embodiments, the single-chain variable fragment (scFv) described above is linked to the antibody constant domain via a hinge sequence. In some embodiments, the hinge comprises amino acids Ala-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G₄S)₄ linker. In some embodiments of the scFv, the heavy chain variable domain is positioned at the N-terminus of the light chain variable domain. In some embodiments of the scFv, the heavy chain variable domain is positioned at the C terminus of the light chain variable domain.

The multi-specific binding proteins described herein can further include one or more additional antigen-binding sites. The additional antigen-binding site(s) may be fused to the N-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multispecific binding protein. For example, a multi-specific binding protein includes an NKG2D-binding site, a B7-H3, L1CAM, FLT1, KDR, TNC, TNN, CSPG4, BST1, SELP, CD200, INSR (HHF5), ITGA6, MELTF, PECAM1, or SLC1A5-binding site, a third antigen-binding site that binds a tumor-associated antigen, and an antibody constant region or a portion thereof sufficient to bind CD16, or a fourth antigen-binding site that binds CD16. Any one of these antigen binding sites can either take the form of an Fab fragment or an scFv, such as the scFv described above. In some embodiments, the third antigen-binding site binds a different tumor-associated antigen from B7-H3, L1CAM, FLT1, KDR, TNC, TNN, CSPG4, BST1, SELP, CD200, INSR (HHF5), ITGA6, MELTF, PECAM1, or SLC1A5. In some embodiments, the third antigen-binding site binds to the same tumor-associated antigen selected from B7-H3, L1CAM, FLT1, KDR, TNC, TNN, CSPG4, BST1, SELP, CD200, INSR (HHF5), ITGA6, MELTF, PECAM1, and SLC1A5. In some embodiments, the third antigen-binding site has the same amino acid sequence(s) as the tumor-associated antigen-binding site that binds a tumor-associated antigen selected from B7-H3, L1CAM, FLT1, KDR, TNC, TNN, CSPG4, BST1, SELP, CD200, INSR (HHF5), ITGA6, MELTF, PECAM1, and SLC1A5. Exemplary formats are shown in FIGS. 2D and 2E. Accordingly, the multi-specific binding proteins can provide bivalent engagement of B7-H3, L1CAM, FLT1, KDR, TNC, TNN, CSPG4, BST1, SELP, CD200, INSR (HHF5), ITGA6, MELTF, PECAM1, or SLC1A5. Bivalent engagement of B7-H3 by the multi-specific proteins can stabilize the B7-H3, L1CAM, FLT1, KDR, TNC, TNN, CSPG4, BST1, SELP, CD200, INSR (HHF5), ITGA6, MELTF, PECAM1, or SLC1A5 on cancer cell surface, and enhance cytotoxicity of NK cells towards the cancer cells. Bivalent engagement of B7-H3, L1CAM, FLT1, KDR, TNC, TNN, CSPG4, BST1, SELP, CD200, INSR (HHF5), ITGA6, MELTF, PECAM1, or SLC1A5 by the multi-specific proteins can confer stronger binding of the multi-specific proteins to the cancer cells, thereby facilitating stronger cytotoxic response of NK cells towards the cancer cells, especially towards cancer cells expressing a low level of B7-H3, L1CAM, FLT1, KDR, TNC, TNN, CSPG4, BST1, SELP, CD200, INSR (HHF5), ITGA6, MELTF, PECAM1, or SLC1A5.

The multi-specific binding proteins can take additional formats. In some embodiments, the multi-specific binding protein is in the Triomab form, which is a trifunctional, bispecific antibody that maintains an IgG-like shape. This chimera consists of two half antibodies, each with one light and one heavy chain, that originate from two parental antibodies.

In some embodiments, the multi-specific binding protein is the KiH form, which involves the knobs-into-holes (KiHs) technology. The KiH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the multi-specific binding protein is in the dual-variable domain immunoglobulin (DVD-Ig™) form, which combines the target binding domains of two monoclonal antibodies via flexible naturally occurring linkers, and yields a tetravalent IgG-like molecule.

In some embodiments, the multi-specific binding protein is in the Orthogonal Fab interface (Ortho-Fab) form. In the ortho-Fab IgG approach (Lewis S M, Wu X, Pustilnik A, Sereno A, Huang F, Rick H L, et al., Generation of bispecific IgG antibodies by structure-based design of an orthogonal Fab interface. Nat. Biotechnol. (2014) 32(2):191-8), structure-based regional design introduces complementary mutations at the LC and HC_{VH-CH1} interface in only one Fab fragment, without any changes being made to the other Fab fragment.

In some embodiments, the multi-specific binding protein is in the 2-in-1 Ig format. In some embodiments, the multi-specific binding protein is in the ES form, which is a heterodimeric construct containing two different Fab fragments binding to targets 1 and target 2 fused to the Fc. Heterodimerization is ensured by electrostatic steering mutations in the Fc.

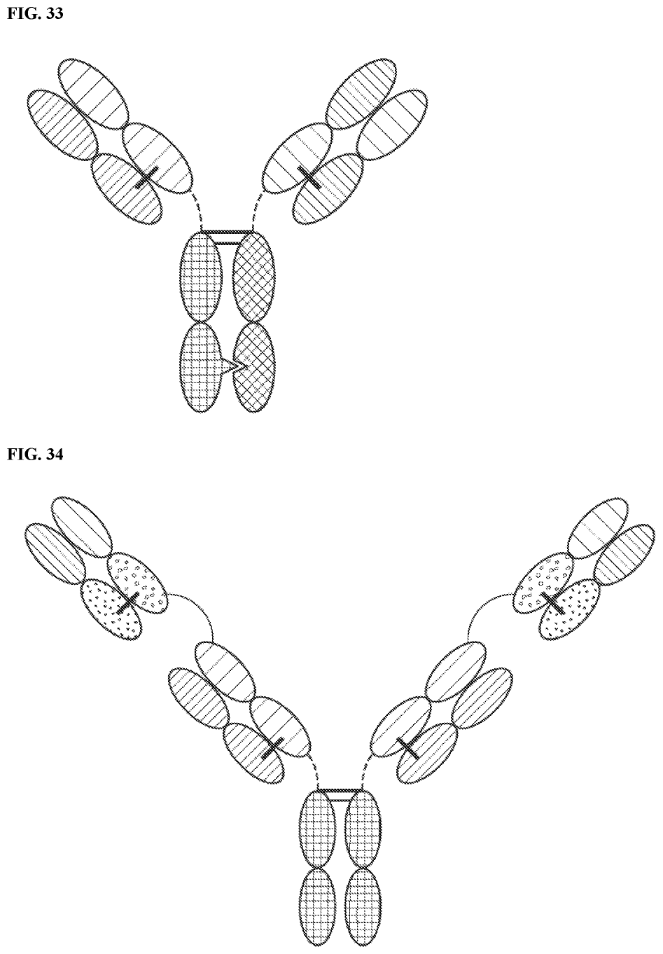

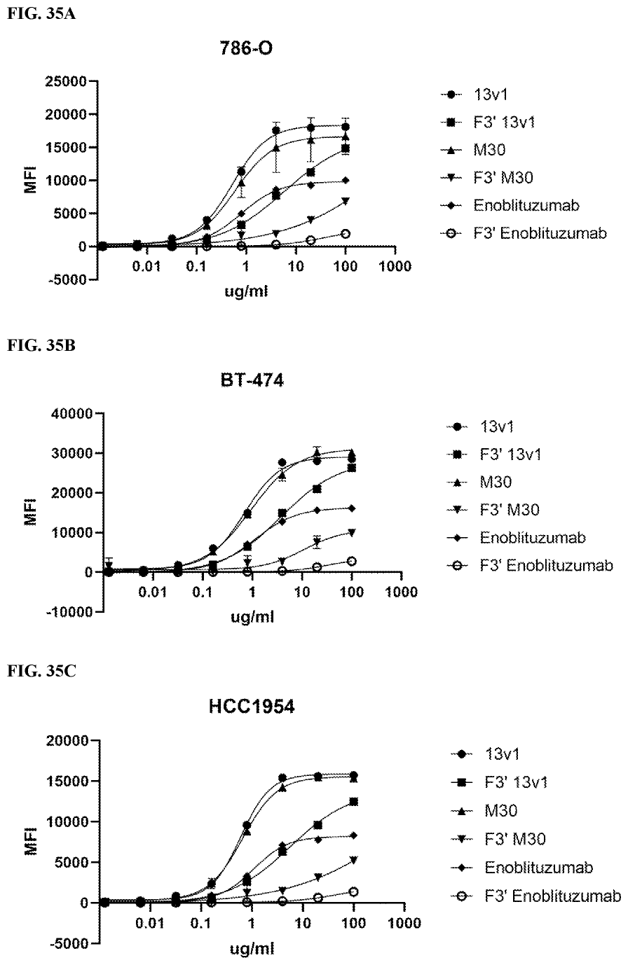

In some embodiments, the multi-specific binding protein is in the κλ-Body form, which is a heterodimeric construct with two different Fab fragments fused to Fc stabilized by heterodimerization mutations: Fab targeting antigen 1 contains kappa LC, while second Fab targeting antigen 2 contains lambda LC. FIG. 30A is an exemplary representation of one form of a κλ-Body; FIG. 30B is an exemplary representation of another κλ-Body.

In some embodiments, the multi-specific binding protein is in Fab Arm Exchange form (antibodies that exchange Fab arms by swapping a heavy chain and attached light chain (half-molecule) with a heavy-light chain pair from another molecule, which results in bispecific antibodies).

In some embodiments, the multi-specific binding protein is in the SEED Body form. The strand-exchange engineered domain (SEED) platform was designed to generate asymmetric and bispecific antibody-like molecules, a capability that expands therapeutic applications of natural antibodies. This protein engineering platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al., Protein Eng. Des. Sel. (2011, 24(5):447-54)).

In some embodiments, the multi-specific binding protein is in the LuZ-Y form, in which a leucine zipper is used to induce heterodimerization of two different HCs. (Wranik, BJ. et al., J. Biol. Chem. (2012), 287:43331-9).

In some embodiments, the multi-specific binding protein is in the Cov-λ-Body form. In bispecific CovX-Bodies, two different peptides are joined together using a branched azetidinone linker and fused to the scaffold antibody under mild conditions in a site-specific manner. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V R et al., PNAS (2010), 107(52); 22611-22616).

In some embodiments, the multi-specific binding protein is in an Oasc-Fab heterodimeric form that includes Fab fragment binding to target 1, and scFab binding to target 2 fused to Fc. Heterodimerization is ensured by mutations in the Fc.

In some embodiments, the multi-specific binding protein is in a DuetMab form, which is a heterodimeric construct containing two different Fab fragments binding to antigens 1 and 2, and Fc stabilized by heterodimerization mutations. Fab fragments 1 and 2 contain differential S-S bridges that ensure correct LC and HC pairing.

In some embodiments, the multi-specific binding protein is in a CrossmAb form, which is a heterodimeric construct with two different Fab fragments binding to targets 1 and 2, fused to Fc stabilized by heterodimerization. CL and CH1 domains and VH and VL domains are switched, e.g., CH1 is fused in-frame with VL, while CL is fused in-frame with VH.

In some embodiments, the multi-specific binding protein is in a Fit-Ig form, which is a homodimeric construct where Fab fragment binding to antigen 2 is fused to the N terminus of HC of Fab fragment that binds to antigen 1. The construct contains wild-type Fc.

Table 1 lists peptide sequences of heavy chain variable domains and light chain variable domains that, in combination, can bind to NKG2D. The NKG2D binding domains can vary in their binding affinity to NKG2D, nevertheless, they all activate human NK cells. Unless indicated otherwise, the CDR sequences provided in Table 1 are determined under Kabat.

TABLE 1

| | Heavy chain variable region | Light chain variable region |
|---|---|---|
| Clones | amino acid sequence | amino acid sequence |
| ADI- | QVQLQQWGAGLLKPSETLSLTCA | DIQMTQSPSTLSASVGDRVTIT |
| 27705 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYNSYPITFGGGTKVEIK |
| | SS | (SEQ ID NO: 2) |
| | (SEQ ID NO: 1) | |
| | CDR1 (SEQ ID NO: 105)- | |
| | GSFSGYYWS | |
| | CDR2 (SEQ ID NO: 106)- | |
| | EIDHSGSTNYNPSLKS | |
| | CDR3 (SEQ ID NO: 107)- | |
| | ARARGPW SFDP | |
| ADI- | QVQLQQWGAGLLKPSETLSLTCA | EIVLTQSPGTLSLSPGERATLS |
| 27724 | VYGGSFSGYYWSWIRQPPGKGLE | CRASQSVSSSYLAWYQQKPG |
| | WIGEIDHSGSTNYNPSLKSRVTIS | QAPRLLIYGASSRATGIPDRFS |
| | VDTSKNQFSLKLSSVTAADTAVY | GSGSGTDFTLTISRLEPEDFAV |
| | YCARARGPWSFDPWGQGTLVTV | YYCQQYGSSPITFGGGTKVEIK |
| | SS | (SEQ ID NO: 4) |
| | (SEQ ID NO: 3) | |
| ADI- | QVQLQQWGAGLLKPSETLSLTCA | DIQMTQSPSTLSASVGDRVTIT |
| 27740 | VYGGSFSGYYWSWIRQPPGKGLE | CRASQSIGSWLAWYQQKPGK |
| (A40) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYHSFYTFGGGTKVEIK |
| | SS | (SEQ ID NO: 6) |
| | (SEQ ID NO: 5) | |
| ADI- | QVQLQQWGAGLLKPSETLSLTCA | DIQMTQSPSTLSASVGDRVTIT |
| 27741 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQSNSYYTFGGGTKVEIK |
| | SS | (SEQ ID NO: 8) |
| | (SEQ ID NO: 7) | |
| ADI- | QVQLQQWGAGLLKPSETLSLTCA | DIQMTQSPSTLSASVGDRVTIT |
| 27743 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYNSYPTFGGGTKVEIK |
| | SS | (SEQ ID NO: 10) |
| | (SEQ ID NO: 9) | |
| ADI- | QVQLQQWGAGLLKPSETLSLTCA | ELQMTQSPSSLSASVGDRVTIT |
| 28153 | VYGGSFSGYYWSWIRQPPGKGLE | CRTSQSISSYLNWYQQKPGQP |
| | WIGEIDHSGSTNYNPSLKSRVTIS | PKLLIYWASTRESGVPDRFSGS |
| | VDTSKNQFSLKLSSVTAADTAVY | GSGTDFTLTISSLQPEDSATYY |
| | YCARARGPWGFDPWGQGTLVTV | CQQSYDIPYTFGQGTKLEIK |
| | SS | (SEQ ID NO: 12) |
| | (SEQ ID NO: 11) | |
| ADI- | QVQLQQWGAGLLKPSETLSLTCA | DIQMTQSPSTLSASVGDRVTIT |
| 28226 | VYGGSFSGYYWSWIRQPPGKGLE | CRASQSISSWLAWYQQKPGK |
| (C26) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYGSFPITFGGGTKVEIK |
| | SS | (SEQ ID NO: 14) |
| | (SEQ ID NO: 13) | |
| ADI- | QVQLQQWGAGLLKPSETLSLTCA | DIQMTQSPSTLSASVGDRVTIT |
| 28154 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTDFTLTISSLQPDDFATY |
| | YCARARGPWSFDPWGQGTLVTV | YCQQSKEVPWTFGQGTKVEIK |
| | SS | (SEQ ID NO: 16) |
| | (SEQ ID NO: 15) | |
| ADI- | QVQLQQWGAGLLKPSETLSLTCA | DIQMTQSPSTLSASVGDRVTIT |
| 29399 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYNSFPTFGGGTKVEIK |
| | SS | (SEQ ID NO: 18) |
| | (SEQ ID NO: 17) | |
| ADI- | QVQLQQWGAGLLKPSETLSLTCA | DIQMTQSPSTLSASVGDRVTIT |
| 29401 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDIYPTFGGGTKVEIK |
| | SS | (SEQ ID NO: 20) |
| | (SEQ ID NO: 19) | |
| ADI- | QVQLQQWGAGLLKPSETLSLTCA | DIQMTQSPSTLSASVGDRVTIT |
| 29403 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDSYPTFGGGTKVEIK |
| | SS | (SEQ ID NO: 22) |
| | (SEQ ID NO: 21) | |
| ADI- | QVQLQQWGAGLLKPSETLSLTCA | DIQMTQSPSTLSASVGDRVTIT |
| 29405 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYGSFPTFGGGTKVEIK |
| | SS | (SEQ ID NO: 24) |
| | (SEQ ID NO: 23) | |
| ADI- | QVQLQQWGAGLLKPSETLSLTCA | DIQMTQSPSTLSASVGDRVTIT |
| 29407 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYQSFPTFGGGTKVEIK |
| | SS | (SEQ ID NO: 26) |
| | (SEQ ID NO: 25) | |
| ADI- | QVQLQQWGAGLLKPSETLSLTCA | DIQMTQSPSTLSASVGDRVTIT |
| 29419 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYSSFSTFGGGTKVEIK |
| | SS | (SEQ ID NO: 28) |
| | (SEQ ID NO: 27) | |
| ADI- | QVQLQQWGAGLLKPSETLSLTCA | DIQMTQSPSTLSASVGDRVTIT |
| 29421 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYESYSTFGGGTKVEIK |
| | SS | (SEQ ID NO: 30) |
| | (SEQ ID NO: 29) | |
| ADI- | QVQLQQWGAGLLKPSETLSLTCA | DIQMTQSPSTLSASVGDRVTIT |
| 29424 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDSFITFGGGTKVEIK |
| | SS | (SEQ ID NO: 32) |
| | (SEQ ID NO: 31) | |
| ADI- | QVQLQQWGAGLLKPSETLSLTCA | DIQMTQSPSTLSASVGDRVTIT |
| 29425 | VYGGSFSGYYWSWIRQPPGKGLE | CRASQSISSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYQSYPTFGGGTKVEIK |
| | SS | (SEQ ID NO: 34) |
| | (SEQ ID NO: 33) | |
| ADI- | QVQLQQWGAGLLKPSETLSLTCA | DIQMTQSPSTLSASVGDRVTIT |
| 29426 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYHSFPTFGGGTKVEIK |
| | SS | (SEQ ID NO: 36) |
| | (SEQ ID NO: 35) | |
| ADI- | QVQLQQWGAGLLKPSETLSLTCA | DIQMTQSPSTLSASVGDRVTIT |
| 29429 | VYGGSFSGYYWSWIRQPPGKGLE | CRASQSIGSWLAWYQQKPGK |
| | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | CARARGPWSFDPWGQGTLVTVSS | YCQQYELYSYTFGGGTKVEIK |
| | (SEQ ID NO: 37) | (SEQ ID NO: 38) |
| ADI- | QVQLQQWGAGLLKPSETLSLTCA | DIQMTQSPSTLSASVGDRVTIT |
| 29447 | VYGGSFSGYYWSWIRQPPGKGLE | CRASQSISSWLAWYQQKPGK |
| (F47) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCQQYDTFITFGGGTKVEIK |
| | SS | (SEQ ID NO: 40) |
| | (SEQ ID NO: 39) | |
| ADI- | QVQLVQSGAEVKKPGSSVKVSCK | DIVMTQSPDSLAVSLGERATIN |
| 27727 | ASGGTFSSYAISWVRQAPGQGLE | CKSSQSVLYSSNNKNYLAWY |
| | WMGGIIPIFGTANYAQKFQGRVTI | QQKPGQPPKLLIYWASTRESG |
| | TADESTSTAYMELSSLRSEDTAV | VPDRFSGSGSGTDFTLTISSLQ |
| | YYCARGDSSIRHAYYYYGMDVW | AEDVAVYYCQQYYSTPITFGG |
| | GQGTTVTVSS | GTKVEIK |
| | (SEQ ID NO: 41) | (SEQ ID NO: 42) |
| | CDR1 (SEQ ID NO: 43)- | CDR1 (SEQ ID NO: 46)- |
| | GTFSSYAIS (non-Kabat) or SYAIS | KSSQSVLYSSNNKNYLA |
| | (SEQ ID NO: 337) | CDR2 (SEQ ID NO: 47)- |
| | CDR2 (SEQ ID NO: 44)- | WASTRES |
| | GIIPIFGTANYAQKFQG | CDR3 (SEQ ID NO: 48)- |
| | CDR3 (SEQ ID NO: 45)- | QQYYSTPIT |
| | ARGDSSIRHAYYYYGMDV (non- | |
| | Kabat) or GDSSIRHAYYYYGMDV | |
| | (SEQ ID NO: 338) | |
| ADI- | QLQLQESGPGLVKPSETLSLTCTV | EIVLTQSPATLSLSPGERATLS |
| 29443 | SGGSISSSSYYWGWIRQPPGKGLE | CRASQSVSRYLAWYQQKPGQ |
| (F43) | WIGSIYYSGSTYYNPSLKSRVTISV | APRLLIYDASNRATGIPARFSG |
| | DTSKNQFSLKLSSVTAADTAVYY | SGSGTDFTLTISSLEPEDFAVY |
| | CARGSDRFHPYFDYWGQGTLVT | YCQQFDTWPPTFGGGTKVEIK |
| | VSS | (SEQ ID NO: 50) |
| | (SEQ ID NO: 49) | CDR1 (SEQ ID NO: 54)- |
| | CDR1 (SEQ ID NO: 51)- | RASQSVSRYLA |
| | GSISSSSYYWG (non-Kabat) or | CDR2 (SEQ ID NO: 55)- |
| | SSSYYWG (SEQ ID NO: 339) | DASNRAT |
| | CDR2 (SEQ ID NO: 52)- | CDR3 (SEQ ID NO: 56)- |
| | SIYYSGSTYYNPSLKS | QQFDTWPPT |
| | CDR3 (SEQ ID NO: 53)- | |
| | ARGSDRFHPYFDY (non-Kabat) or | |
| | GSDRFHPYFDY (SEQ ID NO: 340) | |
| ADI- | QVQLQQWGAGLLKPSETLSLTCA | DIQMTQSPSTLSASVGDRVTIT |
| 29404 | VYGGSFSGYYWSWIRQPPGKGLE | CRASQSISSWLAWYQQKPGK |
| (F04) | WIGEIDHSGSTNYNPSLKSRVTIS | APKLLIYKASSLESGVPSRFSG |
| | VDTSKNQFSLKLSSVTAADTAVY | SGSGTEFTLTISSLQPDDFATY |
| | YCARARGPWSFDPWGQGTLVTV | YCEQYDSYPTFGGGTKVEIK |
| | SS | (SEQ ID NO: 58) |
| | (SEQ ID NO: 57) | |
| ADI- | QVQLVQSGAEVKKPGSSVKVSCK | DIVMTQSPDSLAVSLGERATIN |
| 28200 | ASGGTFSSYAISWVRQAPGQGLE | CESSQSLLNSGNQKNYLTWY |
| | WMGGIIPIFGTANYAQKFQGRVTI | QQKPGQPPKPLIYWASTRESG |
| | TADESTSTAYMELSSLRSEDTAV | VPDRFSGSGSGTDFTLTISSLQ |
| | YYCARRGRKASGSFYYYYGMDV | AEDVAVYYCQNDYSYPYTFG |
| | WGQGTTVTVSS | QGTKLEIK |
| | (SEQ ID NO: 59) | (SEQ ID NO: 60) |
| | CDR1 (SEQ ID NO: 127)- | CDR1 (SEQ ID NO: 130)- |
| | GTFSSYAIS | ESSQSLLNSGNQKNYLT |
| | CDR2 (SEQ ID NO: 128)- | CDR2 (SEQ ID NO: 131)- |
| | GIIPIFGTANYAQKFQG | WASTRES |
| | CDR3 (SEQ ID NO: 129)- | CDR3 (SEQ ID NO: 132)- |
| | ARRGRKASGSFYYYYGMDV | QNDYSYPYT |
| ADI- | QVQLVQSGAEVKKPGASVKVSC | EIVMTQSPATLSVSPGERATLS |
| 29379 | KASGYTFTSYYMHWVRQAPGQG | CRASQSVSSNLAWYQQKPGQ |
| (E79) | LEWMGIINPSGGSTSYAQKFQGR | APRLLIYGASTRATGIPARFSG |
| | VTMTRDTSTSTVYMELSSLRSED | SGSGTEFTLTISSLQSEDFAVY |
| | TAVYYCARGAPNYGDTTHDYYY | YCQQYDDWPFTFGGGTKVEI |
| | MDVWGKGTTVTVSS | K |
| | (SEQ ID NO: 61) | (SEQ ID NO: 62) |
| | CDR1 (SEQ ID NO: 63)- | CDR1 (SEQ ID NO: 66)- |
| | YTFTSYYMH (non-Kabat) or | RASQSVSSNLA |
| | SYYMH (SEQ ID NO: 341) | CDR2 (SEQ ID NO: 67)- |
| | CDR2 (SEQ ID NO: 64) - | GASTRAT |
| | IINPSGGSTSYAQKFQG | CDR3 (SEQ ID NO: 68)- |
| | CDR3 (SEQ ID NO: 65)- | QQYDDWPFT |
| | ARGAPNYGDTTHDYYYMDV | |
| | (non-Kabat) or | |
| | GAPNYGDTTHDYYYMDV (SEQ | |
| | ID NO: 342) | |
| ADI- | QVQLVQSGAEVKKPGASVKVSC | EIVLTQSPGTLSLSPGERATLS |
| 29463 | KASGYTFTGYYMHWVRQAPGQG | CRASQSVSSNLAWYQQKPGQ |
| (F63) | LEWMGWINPNSGGTNYAQKFQG | APRLLIYGASTRATGIPARFSG |
| | RVTMTRDTSISTAYMELSRLRSD | SGSGTEFTLTISSLQSEDFAVY |
| | DTAVYYCARDTGEYYDTDDHGM | YCQQDDYWPPTFGGGTKVEI |
| | DVWGQGTTVTVSS | K |
| | (SEQ ID NO: 69) | (SEQ ID NO: 70) |
| | CDR1 (SEQ ID NO: 71)- | CDR1 (SEQ ID NO: 74)- |
| | YTFTGYYMH (non-Kabat) or | RASQSVSSNLA |
| | GYYMH (SEQ ID NO: 343) | CDR2 (SEQ ID NO: 75)- |
| | CDR2 (SEQ ID NO: 72)- | GASTRAT |
| | WINPNSGGTNYAQKFQG | CDR3 (SEQ ID NO: 76)- |
| | CDR3 (SEQ ID NO: 73)- | QQDDYWPPT |
| | ARDTGEYYDTDDHGMDV (non- | |
| | Kabat) or DTGEYYDTDDHGMDV | |
| | (SEQ ID NO: 344) | |
| ADI- | EVQLLESGGGLVQPGGSLRLSCA | DIQMTQSPSSVSASVGDRVTIT |
| 27744 | ASGFTFSSYAMSWVRQAPGKGLE | CRASQGIDSWLAWYQQKPGK |
| (A44) | WVSAISGSGGSTYYADSVKGRFTI | APKLLIYAASSLQSGVPSRFSG |
| | SRDNSKNTLYLQMNSLRAEDTAV | SGSGTDFTLTISSLQPEDFATY |
| | YYCAKDGGYYDSGAGDYWGQG | YCQQGVSYPRTFGGGTKVEIK |
| | TLVTVSS | (SEQ ID NO: 78) |
| | (SEQ ID NO: 77) | CDR1 (SEQ ID NO: 82)- |
| | CDR1 (SEQ ID NO: 79)- | RASQGIDSWLA |
| | FTFSSYAMS (non-Kabat) or | CDR2 (SEQ ID NO: 83)- |
| | SYAMS (SEQ ID NO: 345) | AASSLQS |
| | CDR2 (SEQ ID NO: 80) - | CDR3 (SEQ ID NO: 84)- |
| | AISGSGGSTYYADSVKG | QQGVSYPRT |
| | CDR3 (SEQ ID NO: 81)- | |
| | AKDGGYYDSGAGDY (non-Kabat) | |
| | or DGGYYDSGAGDY (SEQ ID | |
| | NO: 346) | |
| ADI- | EVQLVESGGGLVKPGGSLRLSCA | DIQMTQSPSSVSASVGDRVTIT |
| 27749 | ASGFTFSSYSMNWVRQAPGKGLE | CRASQGISSWLAWYQQKPGK |
| (A49) | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPMGAAAGWFDPWGQ | YCQQGVSFPRTFGGGTKVEIK |
| | GTLVTVSS | (SEQ ID NO: 86) |
| | (SEQ ID NO: 85) | CDR1 (SEQ ID NO: 90)- |
| | CDR1 (SEQ ID NO: 87)- | RASQGISSWLA |
| | FTFSSYSMN | CDR2 (SEQ ID NO: 91)- |
| | (non-Kabat) or SYSMN (SEQ ID | AASSLQS |
| | NO: 347) CDR2 (SEQ ID NO: 88)- | CDR3 (SEQ ID NO: 92)- |
| | SISSSSSYIYYADSVKG | QQGVSFPRT |
| | CDR3 (SEQ ID NO: 89)- | |
| | ARGAPMGAAAGWFDP (non- | |
| | Kabat) or GAPMGAAAGWFDP | |
| | (SEQ ID NO: 348) | |
| ADI- | QVQLVQSGAEVKKPGASVKVSC | EIVLTQSPATLSLSPGERATLS |
| 29378 | KASGYTFTSYYMHWVRQAPGQG | CRASQSVSSYLAWYQQKPGQ |
| (E78) | LEWMGIINPSGGSTSYAQKFQGR | APRLLIYDASNRATGIPARFSG |
| | VTMTRDTSTSTVYMELSSLRSED | SGSGTDFTLTISSLEPEDFAVY |
| | TAVYYCAREGAGFAYGMDYYY | YCQQSDNWPFTFGGGTKVEIK |
| | MDVWGKGTTVTVS S | (SEQ ID NO: 94) |
| | (SEQ ID NO: 93) | CDR1 (SEQ ID NO: 98)- |
| | CDR1 (SEQ ID NO: 95)- | RASQSVSSYLA |
| | YTFTSYYMH (non-Kabat) or | CDR2 (SEQ ID NO: 99)- |
| | SYYMH (SEQ ID NO: 349) | DASNRAT |
| | CDR2 (SEQ ID NO: 96)- | CDR3 (SEQ ID NO: 100)- |
| | IINPSGGSTSYAQKFQG | QQSDNWPFT |
| | CDR3 (SEQ ID NO: 97)- | |
| | AREGAGFAYGMDYYYMDV (non- | |
| | Kabat) or | |
| | EGAGFAYGMDYYYMDV (SEQ ID | |
| | NO: 350) | |
| A49MI | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPIGAAAGWFDPWGQG | YCQQGVSFPRTFGGGTKVEIK |
| | TLVTVSS (SEQ ID NO: 351) | (SEQ ID NO: 86) |
| | CDR1: FTFSSYSMN (SEQ ID | CDR1 (SEQ ID NO: 90)- |
| | NO: 87) (non-Kabat) or SYSMN | RASQGISSWLA |
| | (SEQ ID NO: 347) | CDR2 (SEQ ID NO: 91)- |
| | CDR2: SISSSSSYIYYADSVKG | AAS SLQS |
| | (SEQ ID NO: 88) | CDR3 (SEQ ID NO: 92)- |
| | CDR3: (non-Kabat) | QQGVSFPRT |
| | ARGAPIGAAAGWFDP (SEQ ID | |
| | NO: 354) or GAPIGAAAGWFDP | |
| | (SEQ ID NO: 352) | |
| A49MQ | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPQGAAAGWFDPWGQ | YCQQGVSFPRTFGGGTKVEIK |
| | GTLVTVS S | (SEQ ID NO: 86) |
| | (SEQ ID NO: 353) | CDR1 (SEQ ID NO: 90)- |
| | CDR1: FTFSSYSMN (SEQ ID | RASQGISSWLA |
| | NO: 87) (non-Kabat) or SYSMN | CDR2 (SEQ ID NO: 91)- |
| | (SEQ ID NO: 347) | AASSLQS |
| | CDR2: SISSSSSYIYYADSVKG | CDR3 (SEQ ID NO: 92)- |
| | (SEQ ID NO: 88) | QQGVSFPRT |
| | CDR3 (non-Kabat) (SEQ ID NO: 385)- | |
| | ARGAPQGAAAGWFDP or CDR3 | |
| | (SEQ ID NO: 355)- | |
| | GAPQGAAAGWFDP | |
| A49ML | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPLGAAAGWFDPWGQ | YCQQGVSFPRTFGGGTKVEIK |
| | GTLVTVSS | (SEQ ID NO: 86) |
| | (SEQ ID NO: 356) | CDR1 (SEQ ID NO: 90)- |
| | CDR1: FTFSSYSMN (SEQ ID | RASQGISSWLA |
| | NO: 87) (non-Kabat) or SYSMN (SEQ | CDR2 (SEQ ID NO: 91)- |
| | ID NO: 347) | AASSLQS |
| | CDR2: SISSSSSYIYYADSVKG | CDR3 (SEQ ID NO: 92)- |
| | (SEQ ID NO: 88) | QQGVSFPRT |
| | CDR3 (non-Kabat) (SEQ ID NO: 357)- | |
| | ARGAPLGAAAGWFDP or CDR3 | |
| | (SEQ ID NO: 358)- | |
| | GAPLGAAAGWFDP | |
| A49MF | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPFGAAAGWFDPWGQ | YCQQGVSFPRTFGGGTKVEIK |
| | GTLVTVSS | (SEQ ID NO: 86) |
| | (SEQ ID NO: 359) | CDR1 (SEQ ID NO: 90)- |
| | CDR1: FTFSSYSMN (SEQ ID | RASQGISSWLA |
| | NO: 87) (non-Kabat) or SYSMN (SEQ | CDR2 (SEQ ID NO: 91)- |
| | ID NO: 347) | AASSLQS |
| | CDR2: SISSSSSYIYYADSVKG | CDR3 (SEQ ID NO: 92)- |
| | (SEQ ID NO: 88) | QQGVSFPRT |
| | CDR3 (non-Kabat) (SEQ ID NO: 360)- | |
| | ARGAPFGAAAGWFDP or CDR3 | |
| | (SEQ ID NO: 361)- | |
| | GAPFGAAAGWFDP | |
| A49MV | EVQLVESGGGLVKPGGSLRLSCA | DIQMTQSPSSVSASVGDRVTIT |
| | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPVGAAAGWFDPWGQ | YCQQGVSFPRTFGGGTKVEIK |
| | GTLVTVSS | (SEQ ID NO: 86) |
| | (SEQ ID NO: 362) | CDR1 (SEQ ID NO: 90)- |
| | CDR1: FTFSSYSMN (SEQ ID | RASQGISSWLA |
| | NO: 87) (non-Kabat) or SYSMN (SEQ | CDR2 (SEQ ID NO: 91)- |
| | ID NO: 347) | AASSLQS |
| | CDR2: SISSSSSYIYYADSVKG | CDR3 (SEQ ID NO: 92)- |
| | (SEQ ID NO: 88) | QQGVSFPRT |
| | CDR3 (non-Kabat) (SEQ ID NO: 363)- | |
| | ARGAPVGAAAGWFDP or CDR3 | |
| | (SEQ ID NO: 364)- | |
| | GAPVGAAAGWFDP | |
| A49- | EVQLVESGGGLVKPGGSLRLSCA | DIQMTQSPSSVSASVGDRVTIT |
| consensus | ASGFTFSSYSMNWVRQAPGKGLE | CRASQGISSWLAWYQQKPGK |
| | WVSSISSSSSYIYYADSVKGRFTIS | APKLLIYAASSLQSGVPSRFSG |
| | RDNAKNSLYLQMNSLRAEDTAV | SGSGTDFTLTISSLQPEDFATY |
| | YYCARGAPXGAAAGWFDPWGQ | YCQQGVSFPRTFGGGTKVEIK |
| | GTLVTVSS, wherein X is M, L, I, | (SEQ ID NO: 86) |
| | V, Q, or F | CDR1 (SEQ ID NO: 90)- |
| | (SEQ ID NO: 365) | RASQGISSWLA |
| | CDR1: FTFSSYSMN (SEQ ID | CDR2 (SEQ ID NO: 91)- |
| | NO: 87) (non-Kabat) or SYSMN (SEQ | AASSLQS |
| | ID NO: 347) | CDR3 (SEQ ID NO: 92)- |
| | CDR2: SISSSSSYIYYADSVKG | QQGVSFPRT |
| | (SEQ ID NO: 88) | |
| | CDR3 (non-Kabat) (SEQ ID NO: 366)- | |
| | ARGAPXGAAAGWFDP or CDR3 | |
| | (SEQ ID NO: 367)- | |
| | GAPXGAAAGWFDP, wherein X is | |
| | M, L, I, V, Q, or F | |

Alternatively, a heavy chain variable domain represented by SEQ ID NO:101 can be paired with a light chain variable domain represented by SEQ ID NO:102 to form an antigen-binding site that can bind to NKG2D, as illustrated in U.S. Pat. No. 9,273,136.

SEQ ID NO: 101
QVQLVESGGGLVKPGGSLRLSCAASGFTFSSYGMEIWVRQAPGKGLEWVA
FIRYDGSNKYYADSVKGRFTISRDNSKNTLYLQMNSLRAEDTAVYYCAKD
RGLGDGTYFDYWGQGTTVTVSS
SEQ ID NO: 102
QSALTQPASVSGSPGQSITISCSGSSSNIGNNAVNWYQQLPGKAPKLLIY
YDDLLPSGVSDRFSGSKSGTSAFLAISGLQSEDEADYYCAAWDDSLNGPV
FGGGTKLTVL

Alternatively, a heavy chain variable domain represented by SEQ ID NO:103 can be paired with a light chain variable domain represented by SEQ ID NO:104 to form an antigen-binding site that can bind to NKG2D, as illustrated in U.S. Pat. No. 7,879,985.

SEQ ID NO: 103
QVHLQESGPGLVKPSETLSLTCTVSDDSISSYYWSWIRQPPGKGLEWIGH
ISYSGSANYNPSLKSRVTISVDTSKNQFSLKLSSVTAADTAVYYCANWDD
AFNIWGQGTMVTVSS
SEQ ID NO: 104
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK

In one aspect, the present disclosure provides multi-specific binding proteins that bind to the NKG2D receptor and CD16 receptor on natural killer cells, and the antigen B7-H3. Table lists some exemplary sequences of heavy chain variable domains and light chain variable domains that, in combination, can bind to B7-H3.

TABLE 2

| | Heavy chain variable domain | Light chain variable domain |
|---|---|---|
| Clones | amino acid sequence | amino acid sequence |
| Enoblituzumab | EVQLVESGGGLVQPGGSLRLS | DIQLTQSPSFLSASVGDRV |
| (MacroGenics, | CAASGFTFSSFGMHWVRQAP | TITCKASQNVDTNVAWY |
| Inc.) | GKGLEWVAYISSDSSAIYYAD | QQKPGKAPKALIYSASYR |
| | TVKGRFTISRDNAKNSLYLQM | YSGVPSRFSGSGSGTDFTL |
| | NSLRDEDTAVYYCGRGR | TISSLQPEDFATYYCQQYN |
| | ENIYYGSRLDYWGQGTTVTVS | NYPFTFGQGTKLEIKR |
| | SA | (SEQ ID NO: 113) |
| | (SEQ ID NO: 109) | CDR1 (SEQ ID NO: 114)- |
| | CDR1 (SEQ ID NO: 110)- | QNVDTNVA |
| | GFTFSSF | CDR1 Chothia (SEQ ID NO: |
| | CDR2 (SEQ ID NO: 111)- | 368)- |
| | SSDSSA | KASQNVDTNVA |
| | CDR3 (SEQ ID NO: 112)- | CDR2 (SEQ ID NO: 115)- |
| | GRENIYYGSRLDY | SASYRYS |
| | | CDR3 (SEQ ID NO: 116)- |
| | | QQYNNYPFT |
| Enoblituzumab | EVQLVESGGGLVQPGGSLRLS | DIQLTQSPSFLSASVGDRV |
| (in scFv | CAASGFTFSSFGMHWVRQAP | TITCKASQNVDTNVAWY |
| construct) | GKCLEWVAYISSDSSAIYYAD | QQKPGKAPKALIYSASYR |
| | TVKGRFTISRDNAKNSLYLQM | YSGVPSRFSGSGSGTDFTL |
| | NSLRDEDTAVYYCGRGRENIY | TISSLQPEDFATYYCQQYN |
| | YGSRLDYWGQGTTVTVSSA | NYPFTFGCGTKLEIK |
| | (SEQ ID NO: 386) | (SEQ ID NO: 387) |
| | CDR1 (SEQ ID NO: 110)- | CDR1 (SEQ ID NO: 114)- |
| | GFTFSSF | QNVDTNVA |
| | CDR2 (SEQ ID NO: 111)- | CDR1 Chothia (SEQ ID NO: |
| | SSDSSA | 368)- |
| | CDR3 (SEQ ID NO: 112)- | KASQNVDTNVA |
| | GRENIYYGSRLDY | CDR2 (SEQ ID NO: 115)- |
| | | SASYRYS |
| | | CDR3 (SEQ ID NO: 116)- |
| | | QQYNNYPFT |
| Omburtamab | QVQLQQSGAELVKPGASVKLS | DIVMTQSPATLSVTPGDR |
| (Y-mAbs | CKASGYTFTNYDINWVRQRPE | VSLSCRASQSISDYLHWY |
| Therapeutics, | QGLEWIGWIFPGDGSTQYNEK | QQKSHESPRLLIKYASQSI |
| Inc.) | FKGKATLTTDTSSSTAYMQLS | SGIPSRFSGSGSGSDFTLSI |
| | RLTSEDSAVYFCARQTTATWF | NSVEPEDVGVYYCQNGHS |
| | AYWGQGTLVTVSAA | FPLTFGAGTKLELKR |
| | (SEQ ID NO: 117) | (SEQ ID NO: 121) |
| | CDR1 (SEQ ID NO: 118)- | CDR1 (SEQ ID NO: 122)- |
| | NYDIN | RASQSISDYLH |
| | CDR2 (SEQ ID NO: 119)- | CDR2 (SEQ ID NO: 123)- |
| | WIFPGDGSTQY | YASQSIS |
| | CDR3 (SEQ ID NO: 120)- | CDR3 (SEQ ID NO: 124)- |
| | QTTATWFAY | QNGHSFPLT |
| huM30 | QVQLVQSGAEVKKPGSSVKV | EIVLTQSPATLSLSPGERA |
| (Daiichi | SCKASGYTFTNYVMHWVRQA | TLSCRASSRLIYMHWYQQ |
| Sankyo, Inc.) | PGQGLEWMGYINPYNDDVKY | KPGQAPRPLIYATSNLASG |
| | NEKFKGRVTITADESTSTAYM | IPARFSGSGSGTDFTLTISS |
| | ELSSLRSEDTAVYYCARWGY | LEPEDFAVYYCQQWNSNP |
| | YGSPLYYFDYWGQGTLVTVS | PTFGQGTKVEIK |
| | S | (SEQ ID NO: 370) |
| | (SEQ ID NO: 369) | CDR1 (SEQ ID NO: 374)- |
| | CDR1 (SEQ ID NO: 371)- | RASSRLIYMH |
| | GYTFTNY | CDR2 (SEQ ID NO: 375)- |
| | CDR2 (SEQ ID NO: 372)- | ATSNLAS |
| | NPYNDD | CDR3 (SEQ ID NO: 376)- |
| | CDR3 (SEQ ID NO: 373)- | QQWNSNPPT |
| | WGYYGSPLYYFDY | |
| huM30 | QVQLVQSGAEVKKPGSSVKV | EIVLTQSPATLSLSPGERA |
| (in scFv | SCKASGYTFTNYVMHWVRQA | TLSCRASSRLIYMHWYQQ |
| construct) | PGQCLEWMGYINPYNDDVKY | KPGQAPRPLIYATSNLASG |
| | NEKFKGRVTITADESTSTAYM | IPARFSGSGSGTDFTLTISS |
| | ELSSLRSEDTAVYYCARWGY | LEPEDFAVYYCQQWNSNP |
| | YGSPLYYFDYWGQGTLVTVS | PTFGCGTKVEIK |
| | S | (SEQ ID NO: 389) |
| | (SEQ ID NO: 388) | CDR1 (SEQ ID NO: 374)- |
| | CDR1 (SEQ ID NO: 371)- | RASSRLIYMH |
| | GYTFTNY | CDR2 (SEQ ID NO: 375)- |
| | CDR2 (SEQ ID NO: 372)- | ATSNLAS |
| | NPYNDD | CDR3 (SEQ ID NO: 376)- |
| | CDR3 (SEQ ID NO: 373)- | QQWNSNPPT |
| | WGYYGSPLYYFDY | |
| huAb 13v1 | EVQLQESGPGLVKPSETLSLTC | DIQMTQSPSSLSASVGDR |
| (AbbVie Inc.) | AVTGYSITSGYSWHWIRQFPG | VTITCKASQNVGFNVAWY |
| US | NGLEWMGYIHSSGSTNYNPSL | QQKPGKSPKALIYSASYR |
| 20170355769 | KSRISISRDTSKNQFFLKLSSVT | YSGVPSRFSGSGSGTDFTL |
| A1 | AADTAVYYCAGYDDYFEYW | TISSLQPEDFAEYFCQQYN |
| | GQGTTVTVSS | WYPFTFGQGTKLEIK |
| | (SEQ ID NO: 377) | (SEQ ID NO: 378) |
| | CDR1 (SEQ ID NO: 379)- | CDR1 (SEQ ID NO: 382)- |
| | GYSITSGY | KASQNVGFNVA |
| | CDR2 (SEQ ID NO: 380)- | CDR2 (SEQ ID NO: 383)- |
| | HSSGS | SASYRYS |
| | CDR3 (SEQ ID NO: 381)- | CDR3 (SEQ ID NO: 384)- |
| | YDDYFEY | QQYNWYPFT |
| huAb 13v1 | EVQLQESGPGLVKPSETLSLTC | DIQMTQSPSSLSASVGDR |
| (in scFv | AVTGYSITSGYSWHWIRQFPG | VTITCKASQNVGFNVAWY |
| construct) | NCLEWMGYIHSSGSTNYNPSL | QQKPGKSPKALIYSASYR |
| | KSRISISRDTSKNQFFLKLSSVT | YSGVPSRFSGSGSGTDFTL |
| | AADTAVYYCAGYDDYFEYW | TISSLQPEDFAEYFCQQYN |
| | GQGTTVTVSS | WYPFTFGCGTKLEIK |
| | (SEQ ID NO: 390) | (SEQ ID NO: 391) |
| | CDR1 (SEQ ID NO: 379)- | CDR1 (SEQ ID NO: 382)- |
| | GYSITSGY | KASQNVGFNVA |
| | CDR2 (SEQ ID NO: 380)- | CDR2 (SEQ ID NO: 383)- |
| | HSSGS | SASYRYS |
| | CDR3 (SEQ ID NO: 381)- | CDR3 (SEQ ID NO: 384)- |
| | YDDYFEY | QQYNWYPFT |

Alternatively, novel antigen-binding sites that can bind to B7-H3 can be identified by screening for binding to the amino acid sequence defined by SEQ ID NO:125 or a mature extracellular fragment thereof.

SEQ ID NO: 125
MLRRRGSPGMGVHVGAALGALWFCLTGALEVQVPEDPVVALVGTDATLC
CSFSPEPGFSLAQLNLIWQLTDTKQLVHSFAEGQDQGSAYANRTALFPD
LLAQGNASLRLQRVRVADEGSFTCFVSIRDFGSAAVSLQVAAPYSKPSM
TLEPNKDLRPGDTVTITCSSYQGYPEAEVFWQDGQGVPLTGNVTTSQMA
NEQGLFDVHSILRVVLGANGTYSCLVRNPVLQQDAHSSVTITPQRSPTG
AVEVQVPEDPVVALVGTDATLRCSFSPEPGFSLAQLNLIWQLTDTKQLV
HSFTEGRDQGSAYANRTALFPDLLAQGNASLRLQRVRVADEGSFTCFVS
IRDFGSAAVSLQVAAPYSKPSMTLEPNKDLRPGDTVTITCSSYRGYPEA
EVFWQDGQGVPLTGNVTTSQMANEQGLFDVHSVLRVVLGANGTYSCLVR
NPVLQQDAHGSVTITGQPMTFPPEALWVTVGLSVCLIALLVALAFVCWR
KIKQSCEEENAGAEDQDGEGEGSKTALQPLKHSDSKEDDGQEIA

In one aspect, the present disclosure provides multi-specific binding proteins that bind to the NKG2D receptor and CD16 receptor on natural killer cells, and the antigen L1CAM. Table 3 lists some exemplary sequences of heavy chain variable domains and light chain variable domains that, in combination, can bind to L1 CAM.

TABLE 3

| | Heavy chain variable domain | Light chain variable domain |
|---|---|---|
| Clones | amino acid sequence | amino acid sequence |
| Patent | EVQLQQSGAELVRPGALVKLS | DIVMTQSQKFMSTSVGDR |
| Publication | CKASGFNIKDYYMQWVKQRP | VSVTCKASQNVGTNVAW |
| No. | EQGLEWIGWIDPENGKTVFDP | YQQKPGHSPKALIYSTSYR |
| US20170306015 | KFRGKASISADTSSNTAYLQLS | YSGVPDRFTGSGSGTDFTL |
| A1 | SLTSEDTAVYYCARWNPLAF | TIRNVQSEDLAEYFCQQY |
| | WGQGTLVTVSS | NTYPYTFGGGTKLEIK |
| | (SEQ ID NO: 133) | (SEQ ID NO: 137) |
| | CDR1 (SEQ ID NO: 134)- | CDR1(SEQ ID NO: 138)- |
| | FNIKDYYMQ | KASQNVGTNVA |
| | CDR2 (SEQ ID NO: 135)- | CDR2 (SEQ ID NO: 139)- |
| | WIDPENGKTVFDPKFRG | STSYRYS |
| | CDR3 (SEQ ID NO: 136)- | CDR3 (SEQ ID NO: 140)- |
| | WNPLAF | QQYNTYPYT |
| Patent | EVQLVESGGGVVQPGRSLRLS | DIQLTQSPSSLSASVGDRV |
| Publication | CAASGFTFSRFGMHWVRQAP | TITCRASRTISIYVNWYRQ |
| No. | GKGLEWVAFISNDGSNKYYA | RPGKAPESLIYAASNLHSG |
| US20150344571 | DSVKGRFTISRDNSKNTLYLQ | VPSRFSGSGSGTDFTLTISS |
| A1 | MNSLRPEDTAVYYCARGRAY | LQPEDFATYYCQQSIGRG |
| | GSGSLFDPWGQGTLVTVSS | VVTFGQGTKLEIK |
| | (SEQ ID NO: 141) | (SEQ ID NO: 145) |
| | CDR1 (SEQ ID NO: 142)- | CDR1 (SEQ ID NO: 146)- |
| | RFGMH | RASRTISIYVN |
| | CDR2 (SEQ ID NO: 143)- | CDR2 (SEQ ID NO: 147)- |
| | FISNDGSNKYYADSVK | AASNLHS |
| | CDR3 (SEQ ID NO: 144)- | CDR3 (SEQ ID NO: 148)- |
| | GRAYGSGSLFDP | QQSIGRGVVT |

Alternatively, novel antigen-binding sites that can bind to L1CAM can be identified by screening for binding to the amino acid sequence defined by SEQ ID NO: 149 or a mature extracellular fragment thereof.

SEQ ID NO: 149
MVVALRYVWPLLLCSPCLLIQIPEEYEGHHVMEPPVITEQSPRRLVVFP
TDDISLKCEASGKPEVQFRWTRDGVHFKPKEELGVTVYQSPHSGSFTIT
GNNSNFAQRFQGIYRCFASNKLGTAMSHEIRLMAEGAPKWPKETVKPVE
VEEGESVVLPCNPPPSAEPLRIYWMNSKILHIKQDERVTMGQNGNLYFA
NVLTSDNHSDYICHAHFPGTRTIIQKEPIDLRVKATNSMIDRKPRLLFP
TNSSSHLVALQGQPLVLECIAEGFPTPTIKWLRPSGPMPADRVTYQNHN
KTLQLLKVGEEDDGEYRCLAENSLGSARHAYYVTVEAAPYWLHKPQSHL
YGPGETARLDCQVQGRPQPEVTWRINGIPVEELAKDQKYRIQRGALILS
NVQPSDTMVTQCEARNRHGLLLANAYIYVVQLPAKILTADNQTYMAVQG
STAYLLCKAFGAPVPSVQWLDEDGTTVLQDERFFPYANGTLGIRDLQAN
DTGRYFCLAANDQNNVTIMANLKVKDATQITQGPRSTIEKKGSRVTFTC
QASFDPSLQPSITWRGDGRDLQELGDSDKYFIEDGRLVIHSLDYSDQGN
YSCVASTELDVVESRAQLLVVGSPGPVPRLVLSDLHLLTQSQVRVSWSP
AEDHNAPIEKYDIEFEDKEMAPEKWYSLGKVPGNQTSTTLKLSPYVHYT
ERVTAINKYGPGEPSPVSETVVTPEAAPEKNPVDVKGEGNETTNMVITW
KPLRWMDWNAPQVQYRVQWRPQGTRGPWQEQIVSDPFLVVSNTSTFVPY
EIKVQAVNSQGKGPEPQVTIGYSGEDYPQAIPELEGIEILNSSAVLVKW
RPVDLAQVKGHLRGYNVTYWREGSQRKHSKRHIHKDHVVVPANTTSVIL
SGLRPYSSYHLEVQAFNGRGSGPASEFTESTPEGVPGHPEALHLECQSN
TSLLLRWQPPLSHNGVLTGYVLSYHPLDEGGKGQLSENLRDPELRTHNL
TDLSPHLRYREQLQATTKEGPGEAIVREGGTMALSGISDEGNISATAGE
NYSVVSWVPKEGQCNERFHILFKALGEEKGGASLSPQYVSYNQSSYTQW
DLQPDTDYEIHLEKERMERHQMAVKTNGTGRVRLPPAGFATEGWFIGFV
SAIILLLLVLLILCFIKRSKGGKYSVKDKEDTQVDSEARPMKDETFGEY
RSLESDNEEKAFGSSQPSLNGDIKPLGSDDSLADYGGSVDVQFNEDGSF
IGQYSGKKEKEAAGGNDSSGATSPINPAVALE

In one aspect, the present disclosure provides multi-specific binding proteins that bind to the NKG2D receptor and CD16 receptor on natural killer cells, and the antigen FLT1, KDR, TNC, TNN, CSPG4, BST1, SELP, CD200, INSR (HHF5), ITGA6, MELTF, PECAM1, or SLC1A5. Table 4 lists some exemplary sequences of heavy chain variable domains and light chain variable domains that, in combination, can bind to FLT1, KDR, TNC, TNN, CSPG4, BST1, SELP, CD200, INSR (HHF5), ITGA6, MELTF, PECAM1, or SLC1A5.

TABLE 4

| | Heavy chain variable domain | Light chain variable domain |
|---|---|---|
| Clones | amino acid sequence | amino acid sequence |
| anti-FLT1 | QAQVVESGGGVVQSGRSLRLS | EIVLTQSPGTLSLSPGERATL |
| (Icrucumab) | CAASGFAFSSYGMHWVRQAP | SCRASQSVSSSYLAWYQQK |
| | GKGLEWVAVIWYDGSNKYY | PGQAPRLLIYGASSRATGIP |
| | ADSVRGRFTISRDNSENTLYL | DRFSGSGSGTDFTLTISRLEP |
| | QMNSLRAEDTAVYYCARDHY | EDFAVYYCQQYGSSPLTFG |
| | GSGVHHYFYYGLDVWGQGTT | GGTKVEIKR |
| | VTVSSA | (SEQ ID NO: 154) |
| | (SEQ ID NO: 150) | CDR1 (SEQ ID NO: 155)- |
| | CDR1 (SEQ ID NO: 151)- | QSVSSSYLA |
| | GFAFSSY | CDR2 (SEQ ID NO: 156)- |
| | CDR2 (SEQ ID NO: 152)- | GASSRAT |
| | WYDGSN | CDR3 (SEQ ID NO: 157)- |
| | CDR3 (SEQ ID NO: 153)- | QQYGSSPLT |
| | DHYGSGVHHYFYYGLDV | |
| anti-FLT1 | QVQLQQSGAELVGPGSSVKIS | DIVMTQSQKFMSTTVGDRV |
| | CKASGYAFSSYWMNWVKQR | SLTCKASQSVGTAVAWYQE |
| | PGQGLEWIGQIYPGDGDTNYN | KTGQSPKLLIYS |
| | GKFRGKVTLTADRSSSTADM | ASNRYTGVPDRFTGSGSGT |
| | QLSSLTSEDSAVYFCARDDGY | DFILTIRNMQSVDLADYFCQ |
| | EGFDYWGQGTTLTVSS | QYFTYPYTFGG |
| | (SEQ ID NO: 158) | GTKLEIQR (SEQ ID NO: 162) |
| | CDRH1: GYAFSSY (SEQ ID | CDRL1: QSVGTAVA (SEQ |
| | NO: 159) | ID NO: 163) |
| | CDRH2: YPGDGD (SEQ ID | CDRL2: SASNRYT (SEQ ID |
| | NO: 160) | NO: 164) |
| | CDRH3: DDGYEGFDY (SEQ ID | CDRL3: QQYFTYPYT (SEQ |
| | NO: 161) | ID NO: 165) |
| KDR | EVQLVQSGGGLVKPGGSLRLS | DIQMTQSPSSVSASIGDRVTI |
| (Amucirumab) | CAASGFTFSSYSMNWVRQAP | TCRASQGIDNWLGWYQQK |
| | GKGLEWVSSISSSSSYIYYADS | PGKAPKLLIYD |
| | VKGRFTISRDNAKNSLYLQMN | ASNLDTGVPSRFSGSGSGTY |
| | SLRAEDTAVYYCARVTDAFDI | FTLTISSLQAEDFAVYFCQQ |
| | WGQGTMVTVSSA (SEQ ID | AKAFPPTFGG |
| | NO: 166) | GTKVDIKG (SEQ ID NO: 170) |
| | CDRH1: GFTFSSY (SEQ ID | CDRL1: QGIDNWLG (SEQ ID |
| | NO: 167) | NO: 171) |
| | CDRH2: SSSSSY (SEQ ID | CDRL2: DASNLDT (SEQ ID |
| | NO: 168) | NO: 172) |
| | CDRH3: VTDAFDI (SEQ ID | CDRL3: QQAKAFPPT (SEQ |
| | NO: 169) | ID NO: 173) |
| KDR | KVQLQQSGTELVKPGASVKVS | DIVLTQSPASLAVSLGQRAT |
| | CKASGYIFTEYIIHWVKQRSG | ISCRASESVDSYGNSFMHW |
| | QGLEWIGWLYPESNIIKYNEK | YQQKPGQPPKL |
| | FKDKATLTADKSSSTVYMELS | LIYRASNLESGIPARFSGSGS |
| | RLTSEDSAVYFCTRHDGTNFD | RTDFTLTINPVEADDVATY |
| | YWGQGTTLTVSSA (SEQ ID | YCQQSNEDPL |
| | NO: 174) | TFGAGTKLELKR |
| | CDRH1: GYIFTEY (SEQ ID | (SEQ ID NO: 178) |
| | NO: 175) | CDRL1: ESVDSYGNSFMH |
| | CDRH2: YPESNI (SEQ ID | (SEQ ID NO: 179) |
| | NO: 176) | CDRL2: RASNLES (SEQ ID |
| | CDRH3: HDGTNFDY (SEQ ID | NO: 180) |
| | NO: 177) | CDRL3: QQSNEDPLT (SEQ |
| | | IDNO: 181) |
| TNC | EIQLQQSGPELVKPGASVKVS | DIVMTQAAPSVPVTPGESVS |
| (tenatumomab) | CKASGYAFTSYNMYWVKQSH | ISCRSSKSLLHSNGNTYLYW |
| | GKSLEWIGYIDPYNGVTSYNQ | FLQRPGQSPQLLIYRMSNLA |
| | KFKGKATLTVDKSSSTAYMH | SGVPDRFSGSGSGTAFTLRI |
| | LNSLTSEDSAVYYCARGGGSI | SRVEAEDVGVYYCMQHLE |
| | YYAMDYWGQGTSVTVSSA | YPLTFGAGTKLELKR |
| | (SEQ ID NO: 182) | (SEQ ID NO: 186) |
| | CDRH1: GYAFTSY (SEQ ID | CDRL1: KSLLHSNGNTYLY |
| | NO: 183) | (SEQ ID NO: 187) |
| | CDRH2: DPYNGV (SEQ ID | CDRL2: RMSNLAS (SEQ ID |
| | NO: 184) | NO: 188) |
| | CDRH3: GGGSIYYAMDY (SEQ | CDRL3: MQHLEYPLT (SEQ |
| | ID NO: 185) | ID NO: 189) |
| TNC | EVQLLESGGGLVQPGGSLRLS | SELTQDPAVSVALGQTVRIT |
| US7968685B2 | CAASGFTFSSYAASWVRQAPG | CQGDSLRSYYASWYQQKP |
| (D5) | KGLEWVSAISGSGGSTYYADS | GQAPVLVIYGKNNRPSGIPD |
| | VKGRFTISRDNSKNTLYLQMN | RFSGSSSGNTASLTITGAQA |
| | SLRAEDTAVYYCAKAHNAFD | EDEADYYCNSSVYTMPPVV |
| | YWGQGTLVTVSR (SEQ ID | FGGGTKLTVLG (SEQ ID |
| | NO: 190) | NO: 194) |
| | CDRH1: SYAAS (SEQ ID | CDRL1: QGDSLRSYYAS |
| | NO: 191) | (SEQ ID NO: 195) |
| | CDRH2: | CDRL2: GKNNRPS (SEQ ID |
| | AISGSGGSTYYADSVK (SEQ | NO: 196) |
| | ID NO: 192) | CDRL3: NSSVYTMPPVV |
| | CDRH3: AHNAFDY (SEQ ID | (SEQ ID NO: 197) |
| | NO: 193) | |
| CSPG4 | AEVQLVESGGGVVRPGGSLRL | EIELTQSPATLSLSPGERATL |
| (US20180072811 | SCAASGFTFDDYGMSWVRQA | SCRASQSVSSYLAWYQQKP |
| A1) | PGKGLEWVSGINWNGGSTGY | GQAPRLLIYDASNRATGIPA |
| | ADSVKGRFTISRDNAKNSLYL | RFSGSGSGTDFTLTISSLEPE |
| | QMNSLRAEDTAVYYCARGVL | DFAVYYCQQRSNWPPAFG |
| | SRYFDYWGQGTLVTVSS | GGTKVEIKR |
| | (SEQ ID NO: 198) | (SEQ ID NO: 202) |
| | CDRH1: GFTFDDYG | CDRL1: QSVSSY |
| | (SEQ ID NO: 199) | (SEQ ID NO: 203) |
| | CDRH2: INWNGGST | CDRL2: DAS |
| | (SEQ ID NO: 200) | (SEQ ID NO: 204) |
| | CDRH3: ARGVLSRYFDY | CDRL3: QQRSNWPPA |
| | (SEQ ID NO: 201) | (SEQ ID NO: 205) |
| CSPG4 | QVQLQESGPGLVKPSQTLSLT | DIQMTQSPSSLSASVGDRVT |
| (US20140242083 | CTVSGGSITSGYYWNWIRQHP | ITCRASQGIRNYLNWYQQK |
| A1 (LC007 M4-3 | GKGLEWIGYITFDGSNNYNPS | PGKAPKLLIYYTSSLHSGVP |
| ML2)) | LKSRVTISRDTSKNQFSLKLSS | SRFSGSGSGTDYTLTISSLQP |
| | VTAADTAVYYCADFDYWGQ | EDFATYYCQQYSALPWTFG |
| | GTLVTVSS | QGTKVEIK (SEQ ID NO: 210) |
| | (SEQ ID NO: 206) | CDRL1: RASQGIRNYLN |
| | CDRH1: SGYYWN | (SEQ ID NO: 211) |
| | (SEQ ID NO: 207) | CDRL2: YTSSLHS |
| | CDRH2: YITFDGSNNYNPSLKS | (SEQ ID NO: 212) |
| | (SEQ ID NO: 208) | CDRL3: QQYSALPWT (SEQ |
| | CDRH3: FDY (SEQ ID NO: 209) | ID NO: 213) |
| BST1 | QAYLQQSGPELVKAGASVKM | DIVMSQSPAIMSASPGEKVT |
| (MEN1112 | SCKASGYSFIEYTINWVKQ | MTCSASSSVTYMYWYQQK |
| (BST1-A2-NF | SHGKSLEWIGNIDPYYGTTYY | PGSSPRLLIYDTSNLASGVP |
| US20160002354 | NQMFTGKATLTVDQSSNTAY | VRFSGSGSGTSYSLTISRME |
| A1)) | MQLKSLTSEDSAVYFCARG | AEDTATYYCQQWSNYPLTF |
| | SAWFPYWGQGTLVTVSA | GAGTKLELK |
| | (SEQ ID NO: 214) | (SEQ ID NO: 218) |
| | CDRH1: GYSFIEYTINW | CDRL1: SASSSVTYMY |
| | (SEQ ID NO: 215) | (SEQ ID NO: 219) |
| | CDRH2: | CDRL2: DTSNLAS |
| | GNIDPYYGTTYYNQMFT | (SEQ ID NO: 220) |
| | (SEQ ID NO: 216) | CDRL3: QQWSNYPLT |
| | CDRH3: ARGSAWFPY | (SEQ ID NO: 221) |
| | (SEQ ID NO: 217) | |
| BST1 | QVQLQQSRAELVMPGASVKM | DIQLTQSPASLSASVGETVTI |
| (BST1-A3 | SCKTSGYTFSDYWVHWVRQR | TCRASENIYSYLAWYQQKQ |
| US20160002354 | PGQGLEWIGAIDGSDTFNDYS | GKSPQLLVYNTKTLGEGVP |
| A1) | QKFKGRATLTVDESSSTVYMQ | SRFSGSGSGTQFSLKINSLQP |
| | LSSLTSEDSAVYYCARG | EDFGSYYCQHHYGTPFTFG |
| | GLLQYWGQGTTLTVSS (SEQ | SGTKLEIK |
| | ID NO: 222) | (SEQ ID NO: 226) |
| | CDRH1: GYTFSDYWVHW | CDRL1: RASENIYSYLA |
| | (SEQ ID NO: 223) | (SEQ ID NO: 227) |
| | CDRH2: | CDRL2: NTKTLGE |
| | GAIDGSDTFNDYSQKFK | (SEQ ID NO: 228) |
| | (SEQ ID NO: 224) | CDRL3: QHHYGTPFT |
| | CDRH3: ARGGLLQY | (SEQ ID NO: 229) |
| | (SEQ ID NO: 225) | |
| SELP | EVQLVESGGGLVRPGGSLRLS | EIVLTQSPATLSLSPGERATL |
| (inclacumab) | CAASGFTFSNYDMHWVRQAT | SCRASQSVSSYLAWYQQKP |
| | GKGLEWVSAITAAGDIYYPGS | GQAPRLLIYDASNRATGIPA |
| | VKGRFTISRENAKNSLYLQMN | RFSGSGSGTDFTLTISSLEPE |
| | SLRAGDTAVYYCARGRYSGS | DFAVYYCQQRSNWPLTFGG |
| | GSYYNDWFDPWGQGTLVTVS | GTKVEIKR |
| | SA (SEQ ID NO: 230) | (SEQ ID NO: 234) |
| | CDRH1: GFTFSNY | CDRL1: QSVSSYLA (SEQ ID |
| | (SEQ ID NO: 231) | NO: 235) |
| | CDRH2: TAAGD | CDRL2: DASNRAT (SEQ ID |
| | (SEQ ID NO: 232) | NO: 236) |
| | CDRH3: | CDRL3: QQRSNWPLT (SEQ |
| | GRYSGSGSYYNDWFDP | ID NO: 237) |
| | (SEQ ID NO: 233) | |
| SELP | QVQLVQSGAEVKKPGASVKV | DIQMTQSPSSLSASVGDRVT |
| (crizanlizumab) | SCKVSGYTFTSYDINWVRQAP | ITCKASQSVDYDGHSYMN |
| | GKGLEWMGWIYPGDGSIKYN | WYQQKPGKAPKLLIYAASN |
| | EKFKGRVTMTVDKSTDTAYM | LESGVPSRFSGSGSGTDFTL |
| | ELSSLRSEDTAVYYCARRGEY | TISSLQPEDFATYYCQQSDE |
| | GNYEGAMDYWGQGTLVTVSS | NPLTFGGGTKVEIKR (SEQ |
| | A (SEQ ID NO: 238) | ID NO: 242) |
| | CDRH1: GYTFTSY (SEQ ID | CDRL1: |
| | NO: 239) | KASQSVDYDGHSYMN (SEQ |
| | CDRH2: YPGDGS (SEQ ID | ID NO: 243) |
| | NO: 240) | CDRL2: AASNLES (SEQ ID |
| | CDRH3: RGEYGNYEGAMDY | NO: 244) |
| | (SEQ ID NO: 241) | CDRL3: QQSDENPLT (SEQ |
| | | ID NO: 245) |
| CD200 | QVQLQQSGSELKKPGASVKIS | DIQMTQSPSSLSASIGDRVTI |
| (samalizumab) | CKASGYSFTDYIILWVRQNPG | TCKASQDINSYLSWFQQKP |
| | KGLEWIGHIDPYYGSSNYNLK | GKAPKLLIYRANRLVDGVP |
| | FKGRVTITADQSTTTAYMELS | SRFSGSGSGTDYTLTISSLQP |
| | SLRSEDTAVYYCGRSKRDYFD | EDFAVYYCLQYDEFPYTFG |
| | YWGQGTTLTVSSA (SEQ ID | GGTKLEIKR (SEQ ID |
| | NO: 246) | NO: 250) |
| | CDRH1: GYSFTDY (SEQ ID | CDRL1: QDINSYLS (SEQ ID |
| | NO: 247) | NO: 251) |
| | CDRH2: DPYYGS (SEQ ID | CDRL2: RANRLVD (SEQ ID |
| | NO: 248) | NO: 252) |
| | CDRH3: SKRDYFDY (SEQ ID | CDRL3: LQYDEFPYT (SEQ |
| | NO: 249) | ID NO: 253) |
| INSR | EVQLVETGGGVVQPGRSLRLS | DVVMTQSPLSLPVTLGQPA |
| (US20170037135 | CAASGFTFSSYAMHWVRQAP | SISCRSSQSLVYGDGNTYLN |
| A1) | GKGLEWVAVISYSGSNKYY | WFQQRPGQSPRRLIYKVSN |
| | ADSVKGRFTISRDNSKNTLYL | RDSGVPDRFSGSGSGTEFTL |
| | QMNSLRAEDTAVYYCARHEW | KISRVEAEDVGVYFCMQGT |
| | GFGFDYWGQGTTVTVSS | YWPGTFGGGTKLEIKRTVA |
| | (SEQ ID NO: 254) | APS (SEQ ID NO: 258) |
| | CDRH1: GFTFSSYA (SEQ ID | CDRL1: QSLVYGDGNTY |
| | NO: 255) | (SEQ ID NO: 259) |
| | CDRH2: ISYSGSNK (SEQ ID | CDRL2: KVS (SEQ ID |
| | NO: 256) | NO: 260) |
| | CDRH3: ARHEWGFGFDY (SEQ | CDRL3: MQGTYWP (SEQ ID |
| | ID NO: 257) | NO: 261) |
| INSR | QVQLQQSGPELVKPGALVKIS | DIQMTQSPSSLSASLGERVS |
| (US20170114152 | CKASGYTFTNYDIHWVKQRP | LTCRASQDIGGNLYWLQQG |
| A1) | GQGLEWIGWIYPGDGSTKYNE | PDGTIKRLIYATSSLDSGVP |
| | KFKGKATLTADKSSSTAYMH | KRFSGSRSGSDYSLTISSLES |
| | LSSLTSEKSAVYFCAREWA | EDFVDYYCLQYSSSPWTFG |
| | YWGQGTLVTVSA | GGTKMEIK |
| | (SEQ ID NO: 262) | (SEQ ID NO: 266) |
| | CDRH1: GYTFTNYDIH (SEQ ID | CDRL1: RASQDIGGNLY |
| | NO: 263) | (SEQ ID NO: 267) |
| | CDRH2: | CDRL2: ATSSLDS (SEQ ID |
| | WIYPGDGSTKYNEKFKG (SEQ | NO: 268) |
| | ID NO: 264) | CDRL3: LQYSSSPWT (SEQ |
| | CDRH3: EWAY (SEQ ID | ID NO: 269) |
| | NO: 265) | |
| ITGA6 | EVQLLESGGGLVQPGGSLRLS | DIQMTQSPSSLSASVGDRVT |
| (a6b4 | CAASGFTFSEYTMSWVRQAP | ITCRASQSISSYLNWYQQKP |
| WO 2008127655) | GKGLEWVSRIYSSGGHTEY | GKAPKLLIYAASSLQSGVPS |
| | ADSVKGRFTISRDNSKNTLYL | RFSGSGSGTDFTLTISSLQPE |
| | QMNSLRAEDTAVYYCAKGSG | DFATYYCQQSYSTPITFGQG |
| | YYHYYYGMDVWGQGTTVTV | TRLEIK (SEQ ID NO: 274) |
| | SS (SEQ ID NO: 270) | CDRL1: RASQSISSYLN (SEQ |
| | CDRH1: EYTMS (SEQ ID | ID NO: 275) |
| | NO: 271) | CDRL2: AASSLQS (SEQ ID |
| | CDRH2: | NO: 276) |
| | RIYSSGGHTEYADSVKG (SEQ | CDRL3: QQSYSTPIT (SEQ ID |
| | ID NO: 272) | NO: 277) |
| | CDRH3: GSGYYHYYYGMDV | |
| | (SEQ ID NO: 273) | |
| ITGA6 | CDRH1: GYYMEI (SEQ ID | CDRL1: RASQSISTWLA |
| (integrin a6b4 | NO: 278) | (SEQ ID NO: 281) |
| US20160194400 | CDRH2: INPSGGTTRLAQKFQ | CDRL2: QASTLTS (SEQ ID |
| A1) | (SEQ ID NO: 279) | NO: 282) |
| | CDRH3: EAHSSGSYFFDY (SEQ | CDRL3: QEYNSYSPWA |
| | ID NO: 280) | (SEQ ID NO: 283) |
| MELTF | QVQLVQSGAEVKKPGASVKV | DIQMTQSPSSLSASVGDRVT |
| US20170320960 | SCKASGYTFTNYRIEWVRQAP | ITCRASQDISNYLNWYQQK |
| A1 | GQGLEWMGEILPRGGNTNY | PGKAPKLLIYYTSRLHSGVP |
| (hSC57.32ss1) | NEKFKGRVTFTADTSTSTAYM | SRFSGSGSGTDYTLTISSLQP |
| | ELRSLRSDDTAVYYCARDDG | EDFATYYCQQGNTLPPTFG |
| | YYGRFAYWGQGTLVTVSS | GGTKVEIK |
| | (SEQ ID NO: 284) | (SEQ ID NO: 288) |
| | CDRH1: NYRIE (SEQ ID | CDRL1: RASQDISNYLN |
| | NO: 285) | (SEQ ID NO: 289) |
| | CDRH2: | CDRL2: YTSRLHS (SEQ ID |
| | EILPRGGNTNYNEKFKG (SEQ | NO: 290) |
| | ID NO: 286) | CDRL3: QQGNTLPPT (SEQ |
| | CDRH3: DDGYYGRFAY (SEQ | ID NO: 291) |
| | ID NO: 287) | |
| MELTF | QVQLVQSGAEVKKPGASVKV | DIQMTQSPSSLSASVGDRVT |
| US20170320960 | SCKASGYTFTNYRIEWVRQAP | ITCRASQDISNYLNWYQQK |
| A1 | GQGLEWMGEILPRGGNTNY | PGKAPKLLIYYTSRLHSGVP |
| (hSC57.32) | NEKFKGRVTFTADTSTSTAYM | SRFSGSGSGTDYTLTISSLQP |
| | ELRSLRSDDTAVYYCARDDG | EDFATYYCQQGNTLPPTFG |
| | YYGRFAYWGQGTLVTVSS | GGTKVEIK |
| | (SEQ ID NO: 292) | (SEQ ID NO: 296) |
| | CDRH1: NYRIE (SEQ ID | CDRL1: RASQDISNYLN |
| | NO: 293) | (SEQ ID NO: 297) |
| | CDRH2: | CDRL2: YTSRLHS (SEQ ID |
| | EILPRGGNTNYNEKFKG (SEQ | NO: 298) |
| | ID NO: 294) | CDRL3: QQGNTLPPT (SEQ |
| | CDRH3: DDGYYGRFAY (SEQ | ID NO: 299) |
| | ID NO: 295) | |
| SLC1A5 | QVQLVQSGSELKKPGAPVKVS | DIQMTQSPSSLSASLGDRVT |
| US20130323789 | CKASGYTFSTFGMSWVRQAP | ITCRASQDIRNYLNWYQQK |
| A1 | GQGLKWMGWIHTYAGVPIY | PGKAPKLLIYYTSRLHSGVP |
| (HV2LV3) | GDDFKGRFVFSLDTSVSTAYL | SRFSGSGSGTDYTLTISSLQP |
| | QISSLKAEDTAVYFCARRSDN | EDFATYFCQQGHTLPPTFG |
| | YRYFFDYWGQGTTVTVSS | QGTKLEIK (SEQ ID NO: 304) |
| | (SEQ ID NO: 300) | CDRL1: RASQDIRNYLN |
| | CDRH1: GYTFSTF (SEQ ID | (SEQ ID NO: 305) |
| | NO: 301) | CDRL2: YTSRLHS (SEQ ID |
| | CDRH2: HTYAGV (SEQ ID | NO: 306) |
| | NO: 302) | CDRL3: QQGHTLPPT (SEQ |
| | CDRH3: RSDNYRYFFDY (SEQ | ID NO: 307) |
| | ID NO: 303) | |
| SLC1A5 | QVQLQQWGAGLLKPSETLSLT | DIQMTQSPSTLSASVGDR |
| WO2018089393 | CAVYGGSFSGYYWSWIRQPP | VTITCRASQSIRSWLAWYQ |
| (germlined | GKGLEWIGEIHHSGGANYNPS | QKPGKAPKLLIYKASILKIG |
| 17cl0) | LKSRVTISVDTSKNQFSLKLSS | VPSRFSGSGSGTEFTLTISSL |
| | VTAADTAVYYCARGQGKNW | QPDDFATYYCQQYYSYSRT |
| | HYDYFDYWGQGTLVTVSSA | FGQGTKVEIK (SEQ ID |
| | (SEQ ID NO: 308) | NO: 312) |
| | CDRH1: GYYWS (SEQ ID | CDRL1: RASQSIRSWLA |
| | NO: 309) | (SEQ ID NO: 313) |
| | CDRH2: EIHHSGGANYNPS | CDRL2: KASILKI (SEQ ID |
| | LKS (SEQ ID NO: 310) | NO: 314) |
| | CDRH3: GQGKNWHYDYFDY | CDRL3: QQYYSYSRT (SEQ |
| | (SEQ ID NO: 311) | ID NO: 315) |

Alternatively, novel antigen-binding sites that can bind to FLT1, KDR, TNC, TNN, CSPG4, BST1, SELP, CD200, INSR (HHF5), ITGA6, MELTF, PECAM1, or SLC1A5 can be identified by screening for binding to the amino acid sequence defined by SEQ ID NO: 316, 317, 318, 319, 320, 321, 322, 323, 324, 325, 326, 327, or 328 respectively. Table 5 lists exemplary sequences of FLT1, KDR, TNC, TNN, CSPG4, BST1, SELP, CD200, INSR (HHF5), ITGA6, MELTF, PECAM1, and SLC1A5. In some embodiments, one or more of SEQ ID NOs: 316-328 are amino acid sequences of preproproteins. A skilled person in the art would appreciate that a preproprotein can be processed into a mature protein in a mammalian cell (e.g., by removing signal peptides and/or cleaving into two or more chains). Accordingly, novel antigen-binding sites that can bind to FLT1, KDR, TNC, TNN, CSPG4, BST1, SELP, CD200, INSR(HHF5), ITGA6, MELTF, PECAM1, or SLC1A5 can also be identified by screening for binding to a mature extracellular fragment of the amino acid sequence defined by SEQ ID NO: 316, 317, 318, 319, 320, 321, 322, 323, 324, 325, 326, 327, or 328, respectively.

TABLE 5

| | | SEQ |
|---|---|---|
| | | ID |
| Antigen | Amino Acid Sequence | NO: |
| FLT1 | MVSYWDTGVLLCALLSCLLLTGSSSGSKLKDPELSLKGTQHIMQA | 316 |
| | GQTLHLQCRGEAAHKWSLPEMVSKESERLSITKSACGRNGKQFCS | |
| | TLTLNTAQANHTGFYSCKYLAVPTSKKKETESAIYIFISDTGRPFVE | |
| | MYSEIPEIIHMTEGRELVIPCRVTSPNITVTLKKFPLDTLIPDGKRIIW | |
| | DSRKGFIISNATYKEIGLLTCEATVNGHLYKTNYLTHRQTNTIIDV | |
| | QISTPRPVKLLRGHTLVLNCTATTPLNTRVQMTWSYPDEKNKRAS | |
| | VRRRIDQSNSHANIFYSVLTIDKMQNKDKGLYTCRVRSGPSFKSV | |
| | NTSVHIYDKAFITVKHRKQQVLETVAGKRSYRLSMKVKAFPSPEV | |
| | VWLKDGLPATEKSARYLTRGYSLIIKDVTEEDAGNYTILLSIKQSN | |
| | VFKNLTATLIVNVKPQIYEKAVSSFPDPALYPLGSRQILTCTAYGIP | |
| | QPTIKWFWHPCNHNHSEARCDFCSNNEESFILDADSNMGNRIESIT | |
| | QRMAIIEGKNKMASTLVVADSRISGIYICIASNKVGTVGRNISFYIT | |
| | DVPNGFHVNLEKMPTEGEDLKLSCTVNKFLYRDVTWILLRTVNN | |
| | RTMHYSISKQKMAITKEHSITLNLTIMNVSLQDSGTYACRARNVY | |
| | TGEEILQKKEITIRDQEAPYLLRNLSDHTVAISSSTTLDCHANGVPE | |
| | PQITWFKNNHKIQQEPGIILGPGSSTLFIERVTEEDEGVYHCKATNQ | |
| | KGSVESSAYLTVQGTSDKSNLELITLTCTCVAATLFWLLLTLFIRK | |
| | MKRSSSEIKTDYLSIIMDPDEVPLDEQCERLPYDASKWEFARERLK | |
| | LGKSLGRGAFGKVVQASAFGIKKSPTCRTVAVKMLKEGATASEY | |
| | KALMTELKILTHIGHHLNVVNLLGACTKQGGPLMVIVEYCKYGN | |
| | LSNYLKSKRDLFFLNKDAALHMEPKKEKMEPGLEQGKKPRLDSV | |
| | TSSESFASSGFQEDKSLSDVEEEEDSDGFYKEPITMEDLISYSFQVA | |
| | RGMEFLSSRKCIHRDLAARNILLSENNVVKICDFGLARDIYKNPDY | |
| | VRKGDTRLPLKWMAPESIFDKIYSTKSDVWSYGVLLWEIFSLGGS | |
| | PYPGVQMDEDFCSRLREGMRMRAPEYSTPEIYQIMLDCWHRDPK | |
| | ERPRFAELVEKLGDLLQANVQQDGKDYIPINAILTGNSGFTYSTPA | |
| | FSEDFFKESISAPKFNSGSSDDVRYVNAFKFMSLERIKTFEELLPNA | |
| | TSMFDDYQGDSSTLLASPMLKRFTWTDSKPKASLKIDLRVTSKSK | |
| | ESGLSDVSRPSFCHSSCGHVSEGKRRFTYDHAELERKIACCSPPPD | |
| | YNSVVLYSTPPI | |
| KDR | MQSKVLLAVALWLCVETRAASVGLPSVSLDLPRLSIQKDILTIKAN | 317 |
| | TTLQITCRGQRDLDWLWPNNQSGSEQRVEVTECSDGLFCKTLTIP | |
| | KVIGNDTGAYKCFYRETDLASVIYVYVQDYRSPFIASVSDQHGVV | |
| | YITENKNKTVVIPCLGSISNLNVSLCARYPEKRFVPDGNRISWDSK | |
| | KGFTIPSYMISYAGMVFCEAKINDESYQSIMYIVVVVGYRIYDVVL | |
| | SPSHGIELSVGEKLVLNCTARTELNVGIDFNWEYPSSKHQHKKLV | |
| | NRDLKTQSGSEMKKFLSTLTIDGVTRSDQGLYTCAASSGLMTKKN | |
| | STFVRVHEKPFVAFGSGMESLVEATVGERVRIPAKYLGYPPPEIKW | |
| | YKNGIPLESNHTIKAGHVLTIMEVSERDTGNYTVILTNPISKEKQSH | |
| | VVSLVVYVPPQIGEKSLISPVDSYQYGTTQTLTCTVYAIPPPHHIH | |
| | WYWQLEEECANEPSQAVSVTNPYPCEEWRSVEDFQGGNKIEVNK | |
| | NQFALIEGKNKTVSTLVIQAANVSALYKCEAVNKVGRGERVISFH | |
| | VTRGPEITLQPDMQPTEQESVSLWCTADRSTFENLTWYKLGPQPL | |
| | PIHVGELPTPVCKNLDTLWKLNATMFSNSTNDILIMELKNASLQD | |
| | QGDYVCLAQDRKTKKRHCVVRQLTVLERVAPTITGNLENQTTSIG | |
| | ESIEVSCTASGNPPPQIMWFKDNETLVEDSGIVLKDGNRNLTIRRV | |
| | RKEDEGLYTCQACSVLGCAKVEAFFIIEGAQEKTNLEIIILVGTAVI | |
| | AMFFWLLLVIILRTVKRANGGELKTGYLSIVMDPDELPLDEHCER | |
| | LPYDASKWEFPRDRLKLGKPLGRGAFGQVIEADAFGIDKTATCRT | |
| | VAVKMLKEGATHSEHRALMSELKILIHIGHHLNVVNLLGACTKPG | |
| | GPLMVIVEFCKFGNLSTYLRSKRNEFVPYKTKGARFRQGKDYVG | |
| | AIPVDLKRRLDSITSSQSSASSGFVEEKSLSDVEEEEAPEDLYKDFL | |
| | TLEHLICYSFQVAKGMEFLASRKCIHRDLAARNILLSEKNVVKICD | |
| | FGLARDIYKDPDYVRKGDARLPLKWMAPETIFDRVYTIQSDVWSF | |
| | GVLLWEIFSLGASPYPGVKIDEEFCRRLKEGTRMRAPDYTTPEMY | |
| | QTMLDCWHGEPSQRPTFSELVEHLGNLLQANAQQDGKDYIVLPIS | |
| | ETLSMEEDSGLSLPTSPVSCMEEEEVCDPKFHYDNTAGISQYLQNS | |
| | KRKSRPVSVKTFEDIPLEEPEVKVIPDDNQTDSGMVLASEELKTLE | |
| | DRTKLSPSFGGMVPSKSRESVASEGSNQTSGYQSGYHSDDTDTTV | |
| | YSSEEAELLKLIEIGVQTGSTAQILQPDSGTTLSSPPV | |
| TNC | MGAMTQLLAGVFLAFLALATEGGVLKKVIRHKRQSGVNATLPEE | 318 |
| | NQPVVFNHVYNIKLPVGSQCSVDLESASGEKDLAPPSEPSESFQEH | |
| | TVDGENQIVFTHRINIPRRACGCAAAPDVKELLSRLEELENLVSSL | |
| | REQCTAGAGCCLQPATGRLDTRPFCSGRGNFSTEGCGCVCEPGW | |
| | KGPNCSEPECPGNCHLRGRCIDGQCICDDGFTGEDCSQLACPSDCN | |
| | DQGKCVNGVCICFEGYAGADCSREICPVPCSEEHGTCVDGLCVCH | |
| | DGFAGDDCNKPLCLNNCYNRGRCVENECVCDEGFTGEDCSELICP | |
| | NDCFDRGRCINGTCYCEEGFTGEDCGKPTCPHACHTQGRCEEGQC | |
| | VCDEGFAGVDCSEKRCPADCHNRGRCVDGRCECDDGFTGADCG | |
| | ELKCPNGCSGHGRCVNGQCVCDEGYTGEDCSQLRCPNDCHSRGR | |
| | CVEGKCVCEQGFKGYDCSDMSCPNDCHQHGRCVNGMCVCDDG | |
| | YTGEDCRDRQCPRDCSNRGLCVDGQCVCEDGFTGPDCAELSCPN | |
| | DCHGQGRCVNGQCVCHEGFMGKDCKEQRCPSDCHGQGRCVDG | |
| | QCICHEGFTGLDCGQHSCPSDCNNLGQCVSGRCICNEGYSGEDCS | |
| | EVSPPKDLVVTEVTEETVNLAWDNEMRVTEYLVVYTPTHEGGLE | |
| | MQFRVPGDQTSTIIQELEPGVEYFIRVFAILENKKSIPVSARVATYL | |
| | PAPEGLKFKSIKETSVEVEWDPLDIAFETWEIIFRNMNKEDEGEITK | |
| | SLRRPETSYRQTGLAPGQEYEISLHIVKNNTRGPGLKRVTTTRLDA | |
| | PSQIEVKDVTDTTALITWFKPLAEIDGIELTYGIKDVPGDRTTIDLT | |
| | EDENQYSIGNLKPDTEYEVSLISRRGDMSSNPAKETFTTGLDAPRN | |
| | LRRVSQTDNSITLEWRNGKAAIDSYRIKYAPISGGDHAEVDVPKSQ | |
| | QATTKTTLTGLRPGTEYGIGVSAVKEDKESNPATINAATELDTPKD | |
| | LQVSETAETSLTLLWKTPLAKFDRYRLNYSLPTGQWVGVQLPRNT | |
| | TSYVLRGLEPGQEYNVLLTAEKGRHKSKPARVKASTEQAPELENL | |
| | TVTEVGWDGLRLNWTAADQAYEHFIIQVQEANKVEAARNLTVPG | |
| | SLRAVDIPGLKAATPYTVSIYGVIQGYRTPVLSAEASTGETPNLGE | |
| | VVVAEVGWDALKLNWTAPEGAYEYFFIQVQEADTVEAAQNLTV | |
| | PGGLRSTDLPGLKAATHYTITIRGVTQDFSTTPLSVEVLTEEVPDM | |
| | GNLTVTEVSWDALRLNWTTPDGTYDQFTIQVQEADQVEEAHNLT | |
| | VPGSLRSMEIPGLRAGTPYTVTLHGEVRGHSTRPLAVEVVTEDLP | |
| | QLGDLAVSEVGWDGLRLNWTAADNAYEHFVIQVQEVNKVEAAQ | |
| | NLTLPGSLRAVDIPGLEAATPYRVSIYGVIRGYRTPVLSAEASTAK | |
| | EPEIGNLNVSDITPESFNLSWMATDGIFETFTIEIIDSNRLLETVEYNI | |
| | SGAERTAHISGLPPSTDFIVYLSGLAPSIRTKTISATATTEALPLLEN | |
| | LTISDINPYGFTVSWMASENAFDSFLVTVVDSGKLLDPQEFTLSGT | |
| | QRKLELRGLITGIGYEVMVSGFTQGHQTKPLRAEIVTEAEPEVDNL | |
| | LVSDATPDGFRLSWTADEGVFDNFVLKIRDTKKQSEPLEITLLAPE | |
| | RTRDITGLREATEYEIELYGISKGRRSQTVSAIATTAMGSPKEVIFS | |
| | DITENSATVSWRAPTAQVESFRITYVPITGGTPSMVTVDGTKTQTR | |
| | LVKLIPGVEYLVSIIAMKGFEESEPVSGSFTTALDGPSGLVTANITD | |
| | SEALARWQPAIATVDSYVISYTGEKVPEITRTVSGNTVEYALTDLE | |
| | PATEYTLRIFAEKGPQKSSTITAKFTTDLDSPRDLTATEVQSETALL | |
| | TWRPPRASVTGYLLVYESVDGTVKEVIVGPDTTSYSLADLSPSTH | |
| | YTAKIQALNGPLRSNMIQTIFTTIGLLYPFPKDCSQAMLNGDTTSG | |
| | LYTIYLNGDKAEALEVFCDMTSDGGGWIVFLRRKNGRENFYQNW | |
| | KAYAAGFGDRREEFWLGLDNLNKITAQGQYELRVDLRDHGETAF | |
| | AVYDKFSVGDAKTRYKLKVEGYSGTAGDSMAYHNGRSFSTFDK | |
| | DTDSAITNCALSYKGAFWYRNCHRVNLMGRYGDNNHSQGVNWF | |
| | HWKGHEHSIQFAEMKLRPSNFRNLEGRRKRA | |
| TNN | MSLQEMFRFPMGLLLGSVLLVASAPATLEPPGCSNKEQQVTVSHT | 319 |
| | YKIDVPKSALVQVDADPQPLSDDGASLLALGEAREEQNIIFRHNIR | |
| | LQTPQKDCELAGSVQDLLARVKKLEEEMVEMKEQCSAQRCCQG | |
| | VTDLSRHCSGHGTFSLETCSCHCEEGREGPACERLACPGACSGHG | |
| | RCVDGRCLCHEPYVGADCGYPACPENCSGHGECVRGVCQCHEDF | |
| | MSEDCSEKRCPGDCSGHGFCDTGECYCEEGFTGLDCAQVVTPQG | |
| | LQLLKNTEDSLLVSWEPSSQVDHYLLSYYPLGKELSGKQIQVPKE | |
| | QHSYEILGLLPGTKYIVTLRNVKNEVSSSPQHLLATTDLAVLGTA | |
| | WVTDETENSLDVEWENPSTEVDYYKLRYGPMTGQEVAEVTVPKS | |
| | SDPKSRYDITGLHPGTEYKITVVPMRGELEGKPILLNGRTEIDSPTN | |
| | VVTDRVTEDTATVSWDPVQAVIDKYVVRYTSADGDTKEMAVHK | |
| | DESSTVLTGLKPGEAYKVYVWAERGNQGSKKADTNALTEIDSPA | |
| | NLVTDRVTENTATISWDPVQATIDKYVVRYTSADDQETREVLVG | |
| | KEQSSTVLTGLRPGVEYTVHVWAQKGDRESKKADTNAPTDIDSP | |
| | KNLVTDRVTENMATVSWDPVQAAIDKYVVRYTSAGGETREVPV | |
| | GKEQSSTVLTGLRPGMEYMVHVWAQKGDQESKKADTKAQTDID | |
| | SPQNLVTDRVTENMATVSWDPVRATIDRYVVRYTSAKDGETREV | |
| | PVGKEQSSTVLTGLRPGVEYTVHVWAQKGAQESKKADTKAQTDI | |
| | DSPQNLVTDWVTENTATVSWDPVQATIDRYVVHYTSANGETREV | |
| | PVGKEQSSTVLTGLRPGMEYTVHVWAQKGNQESKKADTKAQTEI | |
| | DGPKNLVTDWVTENMATVSWDPVQATIDKYMVRYTSADGETRE | |
| | VPVGKEHSSTVLTGLRPGMEYMVHVWAQKGAQESKKADTKAQT | |
| | ELDPPRNLRPSAVTQSGGILTWTPPSAQIHGYILTYQFPDGTVKEM | |
| | QLGREDQRFALQGLEQGATYPVSLVAFKGGRRSRNVSTTLSTVGA | |
| | RFPHPSDCSQVQQNSNAASGLYTIYLHGDASRPLQVYCDMETDG | |
| | GGWIVFQRRNTGQLDFFKRWRSYVEGFGDPMKEFWLGLDKLHN | |
| | LTTGTPARYEVRVDLQTANESAYAIYDFFQVASSKERYKLTVGKY | |
| | RGTAGDALTYHNGWKFTTFDRDNDIALSNCALTHHGGWWYKNC | |
| | HLANPNGRYGETKHSEGVNWEPWKGHEFSIPYVELKIRPHGYSRE | |
| | PVLGRKKRTLRGRLRTF | |
| CSPG4 | MQSGPRPPLPAPGLALALTLTMLARLASAASFFGENHLEVPVATA | 320 |
| | LTDIDLQLQFSTSQPEALLLLAAGPADHLLLQLYSGRLQVRLVLGQ | |
| | EELRLQTPAETLLSDSIPHTVVLTVVEGWATLSVDGFLNASSAVPG | |
| | APLEVPYGLFVGGTGTLGLPYLRGTSRPLRGCLHAATLNGRSLLR | |
| | PLTPDVHEGCAEEFSASDDVALGFSGPHSLAAFPAWGTQDEGTLE | |
| | FTLTTQSRQAPLAFQAGGRRGDFIYVDIFEGHLRAVVEKGQGTVL | |
| | LHNSVPVADGQPHEVSVHINAHRLEISVDQYPTHTSNRGVLSYLEP | |
| | RGSLLLGGLDAEASRHLQEHRLGLTPEATNASLLGCMEDLSVNGQ | |
| | RRGLREALLTRNMAAGCRLEEEEYEDDAYGHYEAFSTLAPEAWP | |
| | AMELPEPCVPEPGLPPVFANFTQLLTISPLVVAEGGTAWLEWRHV | |
| | QPTLDLMEAELRKSQVLFSVTRGARHGELELDIPGAQARKMFTLL | |
| | DVVNRKARFIHDGSEDTSDQLVLEVSVTARVPMPSCLRRGQTYLL | |
| | PIQVNPVNDPPHIIFPHGSLMVILEHTQKPLGPEVFQAYDPDSACEG | |
| | LTFQVLGTSSGLPVERRDQPGEPATEFSCRELEAGSLVYVHRGGPA | |
| | QDLTFRVSDGLQASPPATLKVVAIRPAIQIHRSTGLRLAQGSAMPIL | |
| | PANLSVETNAVGQDVSVLFRVTGALQFGELQKQGAGGVEGAEW | |
| | WATQAFHQRDVEQGRVRYLSTDPQHHAYDTVENLALEVQVGQEI | |
| | LSNLSFPVTIQRATVWMLRLEPLHTQNTQQETLTTAHLEATLEEA | |
| | GPSPPTFHYEVVQAPRKGNLQLQGTRLSDGQGFTQDDIQAGRVTY | |
| | GATARASEAVEDTFRFRVTAPPYFSPLYTFPIHIGGDPDAPVLTNV | |
| | LLVVPEGGEGVLSADHLFVKSLNSASYLYEVMERPRHGRLAWRG | |
| | TQDKTTMVTSFTNEDLLRGRLVYQHDDSETTEDDIPFVATRQGES | |
| | SGDMAWEEVRGVFRVAIQPVNDHAPVQTISRIFHVARGGRRLLTT | |
| | DDVAFSDADSGFADAQLVLTRKDLLFGSIVAVDEPTRPIYRFTQED | |
| | LRKRRVLFVHSGADRGWIQLQVSDGQHQATALLEVQASEPYLRV | |
| | ANGSSLVVPQGGQGTIDTAVLHLDTNLDIRSGDEVHYHVTAGPR | |
| | WGQLVRAGQPATAFSQQDLLDGAVLYSHNGSLSPRDTMAFSVEA | |
| | GPVHTDATLQVTIALEGPLAPLKLVRHKKIYVFQGEAAEIRRDQLE | |
| | AAQEAVPPADIVFSVKSPPSAGYLVMVSRGALADEPPSLDPVQSFS | |
| | QEAVDTGRVLYLHSRPEAWSDAFSLDVASGLGAPLEGVLVELEV | |
| | LPAAIPLEAQNFSVPEGGSLTLAPPLLRVSGPYFPTLLGLSLQVLEP | |
| | PQHGALQKEDGPQARTLSAFSWRMVEEQLIRYVHDGSETLTDSFV | |
| | LMANASEMDRQSHPVAFTVTVLPVNDQPPILTTNTGLQMWEGAT | |
| | APIPAEALRSTDGDSGSEDLVYTIEQPSNGRVVLRGAPGTEVRSFT | |
| | QAQLDGGLVLFSHRGTLDGGFRFRLSDGEHTSPGHFFRVTAQKQV | |
| | LLSLKGSQTLTVCPGSVQPLSSQTLRASSSAGTDPQLLLYRVVRGP | |
| | QLGRLFHAQQDSTGEALVNFTQAEVYAGNILYEHEMPPEPFWEA | |
| | HDTLELQLSSPPARDVAATLAVAVSFEAACPQRPSHLWKNKGLW | |
| | VPEGQRARITVAALDASNLLASVPSPQRSEHDVLFQVTQFPSRGQL | |
| | LVSEEPLHAGQPHFLQSQLAAGQLVYAHGGGGTQQDGFHFRAHL | |
| | QGPAGASVAGPQTSEAFAITVRDVNERPPQPQASVPLRLTRGSRAP | |
| | ISRAQLSVVDPDSAPGEIEYEVQRAPHNGFLSLVGGGLGPVTRFTQ | |
| | ADVDSGRLAFVANGSSVAGIFQLSMSDGASPPLPMSLAVDILPSAI | |
| | EVQLRAPLEVPQALGRSSLSQQQLRVVSDREEPEAAYRLIQGPQY | |
| | GHLLVGGRPTSAFSQFQIDQGEVVFAFTNFSSSHDHFRVLALARG | |
| | VNASAVVNVTVRALLHVWAGGPWPQGATLRLDPTVLDAGELAN | |
| | RTGSVPRFRLLEGPRHGRVVRVPRARTEPGGSQLVEQFTQQDLED | |
| | GRLGLEVGRPEGRAPGPAGDSLTLELWAQGVPPAVASLDFATEPY | |
| | NAARPYSVALLSVPEAARTEAGKPESSTPTGEPGPMASSPEPAVAK | |
| | GGFLSFLEANMFSVIIPMCLVLLLLALILPLLFYLRKRNKTGKHDV | |
| | QVLTAKPRNGLAGDTETFRKVEPGQAIPLTAVPGQGPPPGGQPDP | |
| | ELLQFCRTPNPALKNGQYWV | |
| BST1 | MAAQGCAASRLLQLLLQLLLLLLLLAAGGARARWRGEGTSAHLR | 321 |
| | DIFLGRCAEYRALLSPEQRNKNCTAIWEAFKVALDKDPCSVLPSD | |
| | YDLFINLSRHSIPRDKSLFWENSHLLVNSFADNTRRFNIPLSDVLYG | |
| | RVADFLSWCRQKNDSGLDYQSCPTSEDCENNPVDSFWKRASIQYS | |
| | KDSSGVIHVMLNGSEPTGAYPIKGFFADYEIPNLQKEKITRIEIWV | |
| | MHEIGGPNVESCGEGSMKVLEKRLKDMGFQYSCINDYRPVKLLQ | |
| | CVDHSTHPDCALKSAAAATQRKAPSLYTEQRAGLIIPLFLVLASRT | |
| | QL | |
| SELP | MANCQIAILYQRFQRVVFGISQLLCFSALISELTNQKEVAAWTYHY | 322 |
| | STKAYSWNISRKYCQNRYTDLVAIQNKNEIDYLNKVLPYYSSYY | |
| | WIGIRKNNKTWTWVGTKKALTNEAENWADNEPNNKRNNEDCVE | |
| | IYIKSPSAPGKWNDEHCLKKKHALCYTASCQDMSCSKQGECLETI | |
| | GNYTCSCYPGFYGPECEYVRECGELELPQHVLMNCSHPLGNFSFN | |
| | SQCSFHCTDGYQVNGPSKLECLASGIWTNKPPQCLAAQCPPLKIPE | |
| | RGNMTCLHSAKAFQHQSSCSFSCEEGFALVGPEVVQCTASGVWT | |
| | APAPVCKAVQCQHLEAPSEGTMDCVHPLTAFAYGSSCKFECQPG | |
| | YRVRGLDMLRCIDSGHWSAPLPTCEAISCEPLESPVHGSMDCSPSL | |
| | RAFQYDTNCSFRCAEGFMLRGADIVRCDNLGQWTAPAPVCQALQ | |
| | CQDLPVPNEARVNCSHPFGAFRYQSVCSFTCNEGLLLVGASVLQC | |
| | LATGNWNSVPPECQAIPCTPLLSPQNGTMTCVQPLGSSSYKSTCQF | |
| | ICDEGYSLSGPERLDCTRSGRWTDSPPMCEAIKCPELFAPEQGSLD | |
| | CSDTRGEFNVGSTCHFSCDNGFKLEGPNNVECTTSGRWSATPPTC | |
| | KGIASLPTPGLQCPALTTPGQGTMYCRHHPGTFGFNTTCYFGCNA | |
| | GFTLIGDSTLSCRPSGQWTAVTPACRAVKCSELHVNKPIAMNCSN | |
| | LWGNFSYGSICSFHCLEGQLLNGSAQTACQENGHWSTTVPTCQA | |
| | GPLTIQEALTYFGGAVASTIGLIIVIGGTLLALLRKRFRQKDDGKCPL | |
| | NPHSHLGTYGVFTNAAFDPSP | |
| CD200 | MERLVIRMPFSHLSTYSLVWVMAAVVLCTAQVQVVTQDEREQLY | 323 |
| | TPASLKCSLQNAQEALIVTWQKKKAVSPENMVTFSENHGVVIQPA | |
| | YKDKINITQLGLQNSTITFWNITLEDEGCYMCLFNTFGFGKISGTA | |
| | CLTVYVQPIVSLHYKFSEDHLNITCSATARPAPMVFWKVPRSGIEN | |
| | STVTLSHPNGTTSVTSILHIKDPKNQVGKEVICQVLHLGTVTDFKQ | |
| | TVNKGYWFSVPLLLSIVSLVILLVLISILLYWKRHRNQDRGELSQG | |
| | VQKMT | |
| INSR | MATGGRRGAAAAPLLVAVAALLLGAAGHLYPGEVCPGMDIRNN | 324 |
| | LTRLHELENCSVIEGHLQILLNIFKTRPEDFRDLSFPKLIIVIITDYLLL | |
| | FRVYGLESLKDLFPNLTVIRGSRLFFNYALVIFEMVHLKELGLYNL | |
| | MNITRGSVRIEKNNELCYLATIDWSRILDSVEDNYIVLNKDDNEEC | |
| | GDICPGTAKGKTNCPATVINGQFVERCWTHSHCQKVCPTICKSHG | |
| | CTAEGLCCHSECLGNCSQPDDPTKCVACRNFYLDGRCVETCPPPY | |
| | YHFQDWRCVNFSFCQDLHHKCKNSRRQGCHQYVIHNNKCIPECP | |
| | SGYTMNSSNLLCTPCLGPCPKVCHLLEGEKTIDSVTSAQELRGCTV | |
| | INGSLIINIRGGNNLAAELEANLGLIEEISGYLKIRRSYALVSLSFFR | |
| | KLRLIRGETLEIGNYSFYALDNQNLRQLWDWSKHNLTITQGKLFF | |
| | HYNPKLCLSEIHKMEEVSGTKGRQERNDIALKTNGDQASCENELL | |
| | KFSYIRTSFDKILLRWEPYWPPDFRDLLGFMLFYKEAPYQNVTEFD | |
| | GQDACGSNSWTVVDIDPPLRSNDPKSQNHPGWLMRGLKPWTQY | |
| | AIFVKTLVTFSDERRTYGAKSDIIYVQTDATNPSVPLDPISVSNSSS | |
| | QIILKWKPPSDPNGNITHYLVFWERQAEDSELFELDYCLKGLKLPS | |
| | RTWSPPFESEDSQKHNQSEYEDSAGECCSCPKTDSQILKELEESSFR | |
| | KTFEDYLHNVVFVPRKTSSGTGAEDPRPSRKRRSLGDVGNVTVAV | |
| | PTVAAFPNTSSTSVPTSPEEHRPFEKVVNKESLVISGLRHFTGYRIE | |
| | LQACNQDTPEERCSVAAYVSARTMPEAKADDIVGPVTHEIFENNV | |
| | VHLMWQEPKEPNGLIVLYEVSYRRYGDEELHLCVSRKHFALERG | |
| | CRLRGLSPGNYSVRIRATSLAGNGSWTEPTYFYVTDYLDVPSNIA | |
| | KIIIGPLIFVFLFSVVIGSIYLFLRKRQPDGPLGPLYASSNPEYLSASD | |
| | VFPCSVYVPDEWEVSREKITLLRELGQGSFGMVYEGNARDIIKGE | |
| | AETRVAVKTVNESASLRERIEFLNEASVMKGFTCHHVVRLLGVVS | |
| | KGQPTLVVMELMAHGDLKSYLRSLRPEAENNPGRPPPTLQEMIQ | |
| | MAAEIADGMAYLNAKKFVHRDLAARNCMVAHDFTVKIGDFGMT | |
| | RDIYETDYYRKGGKGLLPVRWMAPESLKDGVFTTSSDMWSFGVV | |
| | LWEITSLAEQPYQGLSNEQVLKFVMDGGYLDQPDNCPERVTDLM | |
| | RMCWQFNPKMRPTFLEIVNLLKDDLHPSFPEVSFFHSEENKAPESE | |
| | ELEMEFEDMENVPLDRSSHCQREEAGGRDGGSSLGFKRSYEEHIP | |
| | YTHMNGGKKNGRILTLPRSNPS | |
| ITGA6 | MAAAGQLCLLYLSAGLLSRLGAAFNLDTREDNVIRKYGDPGSLFG | 325 |
| | FSLAMHWQLQPEDKRLLLVGAPRAEALPLQRANRTGGLYSCDIT | |
| | ARGPCTRIEFDNDADPTSESKEDQWMGVTVQSQGPGGKVVTCAH | |
| | RYEKRQHVNTKQESRDIFGRCYVLSQNLRIEDDMDGGDWSFCDG | |
| | RLRGHEKFGSCQQGVAATFTKDFHYIVFGAPGTYNWKGIVRVEQ | |
| | KNNTFFDMNIFEDGPYEVGGETEHDESLVPVPANSYLGLLFLTSVS | |
| | YTDPDQFVYKTRPPREQPDTFPDVMMNSYLGFSLDSGKGIVSKDE | |
| | ITFVSGAPRANHSGAVVLLKRDMKSAHLLPEHIFDGEGLASSFGY | |
| | DVAVVDLNKDGWQDIVIGAPQYFDRDGEVGGAVYVYMNQQGR | |
| | WNNVKPIRLNGTKDSMFGIAVKNIGDINQDGYPDIAVGAPYDDLG | |
| | KVFIYHGSANGINTKPTQVLKGISPYFGYSIAGNMDLDRNSYPDVA | |
| | VGSLSDSVTIFRSRPVINIQKTITVTPNRIDLRQKTACGAPSGICLQV | |
| | KSCFEYTANPAGYNPSISIVGTLEAEKERRKSGLSSRVQFRNQGSE | |
| | PKYTQELTLKRQKQKVCMEETLWLQDNIRDKLRPIPITASVEIQEP | |
| | SSRRRVNSLPEVLPILNSDEPKTAHIDVHFLKEGCGDDNVCNSNLK | |
| | LEYKFCTREGNQDKFSYLPIQKGVPELVLKDQKDIALEITVTNSPS | |
| | NPRNPTKDGDDAHEAKLIATFPDTLTYSAYRELRAFPEKQLSCVA | |
| | NQNGSQADCELGNPFKRNSNVTFYLVLSTTEVTFDTPDLDINLKLE | |
| | TTSNQDNLAPITAKAKVVIELLLSVSGVAKPSQVYFGGTVVGEQA | |
| | MKSEDEVGSLIEYEFRVINLGKPLTNLGTATLNIQWPKEISNGKWL | |
| | LYLVKVESKGLEKVTCEPQKEINSLNLTESHNSRKKREITEKQIDD | |
| | NRKFSLFAERKYQTLNCSVNVNCVNIRCPLRGLDSKASLILRSRLW | |
| | NSTFLEEYSKLNYLDILMRAFIDVTAAAENIRLPNAGTQVRVTVFP | |
| | SKTVAQYSGVPWWIILVAILAGILMLALLVFILWKCGFFKRSRYD | |
| | DSVPRYHAVRIRKEEREIKDEKYIDNLEKKQWITKWNENESYS | |
| MELTF | MRGPSGALWLLLALRTVLGGMEVRWCATSDPEQHKCGNMSEAF | 326 |
| | REAGIQPSLLCVRGTSADHCVQLIAAQEADAITLDGGAIYEAGKEH | |
| | GLKPVVGEVYDQEVGTSYYAVAVVRRSSHVTIDTLKGVKSCHTGI | |
| | NRTVGWNVPVGYLVESGRLSVMGCDVLKAVSDYFGGSCVPGAG | |
| | ETSYSESLCRLCRGDSSGEGVCDKSPLERYYDYSGAFRCLAEGAG | |
| | DVAFVKHSTVLENTDGKTLPSWGQALLSQDFELLCRDGSRADVT | |
| | EWRQCHLARVPAHAVVVRADTDGGLIFRLLNEGQRLFSHEGSSFQ | |
| | MFSSEAYGQKDLLFKDSTSELVPIATQTYEAWLGHEYLHAMKGL | |
| | LCDPNRLPPYLRWCVLSTPEIQKCGDMAVAFRRQRLKPEIQCVSA | |
| | KSPQHCMERIQAEQVDAVTLSGEDIYTAGKTYGLVPAAGEHYAPE | |
| | DSSNSYYVVAVVRRDSSHAFTLDELRGKRSCHAGFGSPAGWDVP | |
| | VGALIQRGFIRPKDCDVLTAVSEFFNASCVPVNNPKNYPSSLCALC | |
| | VGDEQGRNKCVGNSQERYYGYRGAFRCLVENAGDVAFVRHTTV | |
| | FDNTNGHNSEPWAAELRSEDYELLCPNGARAEVSQFAACNLAQIP | |
| | PHAVMVRPDTNIFTVYGLLDKAQDLFGDDHNKNGFKMFDSSNYH | |
| | GQDLLFKDATVRAVPVGEKTTYRGWLGLDYVAALEGMSSQQCS | |
| | GAAAPAPGAPLLPLLLPALAARLLPPAL | |
| PECAM1 | MQPRWAQGATMWLGVLLTLLLCSSLEGQENSFTINSVDMKSLPD | 327 |
| | WTVQNGKNLTLQCFADVSTTSHVKPQHQMLFYKDDVLFYNISSM | |
| | KSTESYFIPEVRIYDSGTYKCTVIVNNKEKTTAEYQVLVEGVPSPR | |
| | VTLDKKEAIQGGIVRVNCSVPEEKAPIHFTIEKLELNEKMVKLKRE | |
| | KNSRDQNFVILEFPVEEQDRVLSFRCQARIISGIHMQTSESTKSELV | |
| | TVTESFSTPKFHISPTGMIMEGAQLHIKCTIQVTHLAQEFPEIIIQKD | |
| | KAIVAHNRHGNKAVYSVMAMVEHSGNYTCKVESSRISKVSSIVV | |
| | NITELFSKPELESSFTHLDQGERLNLSCSIPGAPPANFTIQKEDTIVS | |
| | QTQDFTKIASKSDSGTYICTAGIDKVVKKSNTVQIVVCEMLSQPRI | |
| | SYDAQFEVIKGQTIEVRCESISGTLPISYQLLKTSKVLENSTKNSND | |
| | PAVFKDNPTEDVEYQCVADNCHSHAKMLSEVLRVKVIAPVDEVQ | |
| | ISILSSKVVESGEDIVLQCAVNEGSGPITYKFYREKEGKPFYQMTSN | |
| | ATQAFWTKQKASKEQEGEYYCTAFNRANHASSVPRSKILTVRVIL | |
| | APWKKGLIAVVIIGVIIALLIIAAKCYFLRKAKAKQMPVEMSRPAV | |
| | PLLNSNNEKMSDPNMEANSHYGHNDDVRNHAMKPINDNKEPLNS | |
| | DVQYTEVQVSSAESHKDLGKKDTETVYSEVRKAVPDAVESRYSR | |
| | TEGSLDGT | |
| SLC1A5 | MVADPPRDSKGLAAAEPTANGGLALASIEDQGAAAGGYCGSRDQ | 328 |
| | VRRCLRANLLVLLTVVAVVAGVALGLGVSGAGGALALGPERLSA | |
| | FVFPGELLLRLLRMIILPLVVCSLIGGAASLDPGALGRLGAWALLF | |
| | FLVTTLLASALGVGLALALQPGAASAAINASVGAAGSAENAPSKE | |
| | VLDSFLDLARNIFPSNLVSAAFRSYSTTYEERNITGTRVKVPVGQE | |
| | VEGMNILGLVVFAIVFGVALRKLGPEGELLIRFFNSFNEATMVLVS | |
| | WIMWYAPVGIMFLVAGKIVEMEDVGLLFARLGKYILCCLLGHAIH | |
| | GLLVLPLIYFLFTRKNPYRFLWGIVTPLATAFGTSSSSATLPLMMK | |
| | CVEENNGVAKHISRFILPIGATVNMDGAALFQCVAAVFIAQLSQQS | |
| | LDFVKIITILVTATASSVGAAGIPAGGVLTLAIILEAVNLPVDHISLIL | |
| | AVDWLVDRSCTVLNVEGDALGAGLLQNYVDRTESRSTEPELIQV | |
| | KSELPLDPLPVPTEEGNPLLKHYRGPAGDATVASEKESVM | |

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as a human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, 5354, Q352, E356, E357, K360, Q362, 5364, T366, L368, K370, N390, K392, T394, D399, 5400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, Q347R, S354C, E356K, E357Q, E357L, E357W, K360E, K360W, Q362E, S364K, S364E, S364H, S364D, T366V, T366, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, F405L, Y407A, Y4071, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 6.

TABLE 6

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 7.

TABLE 7

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of sub situations shown in Table 8.

TABLE 8

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 9.

TABLE 9

| First Polypeptide | Second Polypeptide |
|---|---|
| L351Y, D399R, D399K, | T366V, T3661, T366L, |
| S400K, S400R, Y407A, | T366M, N390D, N390E, |
| Y4071, Y407V | K392L, K392M, K392V, |
| | K392F K392D, K392E, |
| | K409F, K409W, T411D and |
| | T411E |

Alternatively, at least one amino acid substitutions could be selected from the following set of substitutions in Table 10, where the position(s) indicated in the First Polypeptide column is replaced by any known negatively-charged amino acid, and the position(s) indicated in the Second Polypeptide Column is replaced by any known positively-charged amino acid.

TABLE 10

| First Polypeptide | Second Polypeptide |
|---|---|
| K392, K370, K409, or K439 | D399, E356, or E357 |

Alternatively, at least one amino acid substitutions could be selected from the following set of in Table 11, where the position(s) indicated in the First Polypeptide column is replaced by any known positively-charged amino acid, and the position(s) indicated in the Second Polypeptide Column is replaced by any known negatively-charged amino acid.

TABLE 11

| First Polypeptide | Second Polypeptide |
|---|---|
| D399, E356, or E357 | K409, K439, K370, or K392 |

Alternatively, amino acid substitutions could be selected from the following set in Table 12.

TABLE 12

| First Polypeptide | Second Polypeptide |
|---|---|
| T350V, L351Y, F405A, and | T350V, T366L, K392L, and |
| Y407V | T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitution and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

### Exemplary Multi-Specific Binding Proteins

A TriNKET of the present disclosure is A49MI-F3′-TriNKET-Enoblituzumab, sequences of which are provided below (CDRs (Chothia numbering) are underlined).

A49MI-F3′-TriNKET-Enoblituzumab includes a single-chain variable fragment (scFv) (SEQ ID NO:329) derived from enoblituzumab that binds B7-H3, linked to an Fc domain via a hinge comprising Ala-Ser (the sequence of the scFv-Fc polypeptide is represented by SEQ ID NO:330); and an NKG2D-binding Fab fragment derived from A49MI including a heavy chain portion comprising a heavy chain variable domain (SEQ ID NO:351) and a CH1 domain, and a light chain portion comprising a light chain variable domain (SEQ ID NO:86) and a light chain constant domain, wherein the heavy chain variable domain is connected to the CH1 domain, and the CH1 domain is connected to an Fc domain that forms a dimer with the Fc domain that is linked to the B7-H3 binding scFv.

The B7-H3-binding scFv includes a heavy chain variable domain of enoblituzumab connected to a light chain variable domain of enoblituzumab with a (G₄S)₄ linker. The heavy and the light variable domains of the scFv (SEQ ID NO:329) are connected as VL-(G4S)₄-VH; VL and VH contain 100VL-44VH S-S bridge as a result of Q100C and G44C substitutions, respectively. In the amino acid sequences of SEQ ID NO:329 and SEQ ID NO:330 below, the cysteine residues are bold-italics-underlined, and the (G₄S)₄ linker (GGGGSGGGGSGGGGSGGGGS, SEQ ID NO:126) is bold-underlined.

Enoblituzumab scFv
(SEQ ID NO: 329)
DIQLTQSPSFLSASVGDRVTITCKASQNVDTNVAWYQQKPGKAPKALIYSA
SYRYSGVPSRFSGSGSGTDFTLTISSLQPEDFATYYCQQYNNYPFTFG [symbol] GT
KLEIKGGGGSGGGGSGGGGSGGGGSEVQLVESGGGLVQPGGSLRLSCAASG
FTFSSFGMHWVRQAPGK [symbol] LEWVAYISSDSSAIYYADTVKGRFTISRDNAKN
SLYLQMNSLRDEDTAVYYCGRGRENIYYGSRLDYWGQGTTVTVSS

SEQ ID NO:330 represents the full sequence of a B7-H3-binding scFv linked to an Fe domain via a hinge comprising Ala-Ser (scFv-Fc). The Fe domain linked to the scFv includes Q347R, D399V, and F405T substitutions, which are bold-underlined in the sequence below. This Fc domain further includes an S354C substitution, which is bold-italics-underlined.

Enoblituzumab scFv-Fc
(SEQ ID NO: 330)
DIQLTQSPSFLSASVGDRVTITCKASQNVDTNVAWYQQKPGKAPKALIYSA
SYRYSGVPSRFSGSGSGTDFTLTISSLQPEDFATYYCQQYNNYPFTFG [symbol] GT
KLEIKGGGGSGGGGSGGGGSGGGGSEVQLVESGGGLVQPGGSLRLSCAASG
FTFSSFGMHWVRQAPGK [symbol] LEWVAYISSDSSAIYYADTVKGRFTISRDNAKN
SLYLQMNSLRDEDTAVYYCGRGRENIYYGSRLDYWGQGTTVTVSSASDKTH
TCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNK
ALPAPIEKTISKAKGQPREPRVYTLPP [symbol] RDELTKNQVSLTCLVKGFYPSDI
AVEWESNGQPENNYKTTPPVLVSDGSFTLYSKLTVDKSRWQQGNVFSCSVM
HEALHNHYTQKSLSLSPG

SEQ ID NO:331 represents the heavy chain portion of an Fab fragment derived from A49MI, which comprises a heavy chain variable domain (SEQ ID NO:351) of an NKG2D-binding site and a CH1 domain, connected to an Fc domain. The Fc domain in SEQ ID NO:331 includes a Y349C substitution in the CH3 domain, which forms a disulfide bond with the S354C substitution on the Fc linked to the B7-H3-binding scFv (SEQ ID NO:330). In SEQ ID NO:331, the Fc domain also includes K360E and K409W substitutions.

A49MI V_{H}
(SEQ ID NO: 351)
EVQLVESGGGLVKPGGSLRLSCAASGFTFSSYSMNWVRQAPGKGLEWVSSI
SSSSSYIYYADSVKGRFTISRDNAKNSLYLQMNSLRAEDTAVYYCARGAPI
GAAAGWFDPWGQGTLVTVSS
A49MI V_{H}-CH1-Fc
(SEQ ID NO: 331)
EVQLVESGGGLVKPGGSLRLSCAASGFTFSSYSMNWVRQAPGKGLEWVSSI
SSSSSYIYYADSVKGRFTISRDNAKNSLYLQMNSLRAEDTAVYYCARGAPI
GAAAGWFDPWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQV [symbol] TLP
PSRDELTENQVSLTCLVKGFYPSDIAVEWESNGQPENNYKTTPPVLDSDGS
FFLYSWLTVDKSRWQQGNVFSCSVMHEALHNHYTQKSLSLSPG

SEQ ID NO:332 represents the light chain portion of an Fab fragment derived from A49MI, the sequence comprising a light chain variable domain (SEQ ID NO:86) of an NKG2D-binding site and a light chain constant domain.

A49MI V_{L}
(SEQ ID NO: 86)
DIQMTQSPSSVSASVGDRVTITCRASQGISSWLAWYQQKPGKAPKLLIYAA
SSLQSGVPSRFSGSGSGTDFTLTISSLQPEDFATYYCQQGVSFPRTFGGGT
KVEIK
A49MI V_{L}-CL
(SEQ ID NO: 332)
DIQMTQSPSSVSASVGDRVTITCRASQGISSWLAWYQQKPGKAPKLLIYAA
SSLQSGVPSRFSGSGSGTDFTLTISSLQPEDFATYYCQQGVSFPRTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

In certain embodiments, a TriNKET of the present disclosure is identical to A49MI-F3′-TriNKET-Enoblituzumab, except that the Fc domain linked to the NKG2D-binding Fab fragment comprises the substitutions of Q347R, D399V, and F405T, and the Fc domain linked to the Enoblituzumab scFv comprises matching substitutions K360E and K409W for forming a heterodimer. In certain embodiments, a TriNKET of the present disclosure is identical to A49MI-F3′-TriNKET-Enoblituzumab, except that the Fc domain linked to the NKG2D-binding Fab fragment includes an S354C substitution in the CH3 domain, and the Fc domain linked to the Enoblituzumab scFv includes a matching Y349C substitution in the CH3 domain for forming a disulfide bond.

Another TriNKET of the present disclosure is A49MI-F3′-TriNKET-huM30, sequences of which are provided below (CDRs (Chothia numbering) are underlined).

A49MI-F3′-TriNKET-huM30 includes an scFv (SEQ ID NO:335) derived from huM30 that binds B7-H3, linked to an Fc domain via a hinge comprising Ala-Ser (the sequence of the scFv-Fc polypeptide is represented by SEQ ID NO:336); and an NKG2D-binding Fab fragment derived from A49MI including a heavy chain portion comprising a heavy chain variable domain (SEQ ID NO:351) and a CH1 domain, and a light chain portion comprising a light chain variable domain (SEQ ID NO:86) and a light chain constant domain, wherein the heavy chain variable domain is connected to the CH1 domain, and the CH1 domain is connected to an Fe domain that forms a dimer with the Fe domain that is linked to the B7-H3 binding scFv.

The B7-H3-binding scFv includes a heavy chain variable domain of huM30 connected to a light chain variable domain of huM30 with a (G₄S)₄ linker. The heavy and the light variable domains of the scFv (SEQ ID NO:335) are connected as VL-(G4S)₄—VH; VL and VH contain 100VL-44VH S-S bridge as a result of Q100C and G44C substitutions, respectively. In the amino acid sequences of SEQ ID NO:335 and SEQ ID NO:336 below, the cysteine residues are bold-italics-underlined, and the (G₄S)₄ linker (GGGGSGGGGSGGGGSGGGGS, SEQ ID NO:126) is bold-underlined.

huM30 scFv
(SEQ ID NO: 335)
EIVLTQSPATLSLSPGERATLSCRASSRLIYMHWYQQKPGQAPRPLIYATS
NLASGIPARFSGSGSGTDFTLTISSLEPEDFAVYYCQQWNSNPPTFG [symbol] GTK
VEIKGGGGSGGGGSGGGGSGGGGSQVQLVQSGAEVKKPGSSVKVSCKASGY
TFTNYVMHWVRQAPGQCLEWMGYINPYNDDVKYNEKFKGRVTITADESTST
AYMELSSLRSEDTAVYYCARWGYYGSPLYYFDYWGQGTLVTVSS

SEQ ID NO:336 represents the full sequence of a B7-H3-binding scFv linked to an Fc domain via a hinge comprising Ala-Ser (scFv-Fc). The Fc domain linked to the scFv includes Q347R, D399V, and F405T substitutions, which are bold-underlined in the sequence below. This Fc domain further includes an S354C substitution, which is bold-italics-underlined.

huM30 scFv-Fc
(SEQ ID NO: 336)
EIVLTQSPATLSLSPGERATLSCRASSRLIYMHWYQQKPGQAPRPLIYATS
NLASGIPARFSGSGSGTDFTLTISSLEPEDFAVYYCQQWNSNPPTFG [symbol] GTK
VEIKGGGGSGGGGSGGGGSGGGGSQVQLVQSGAEVKKPGSSVKVSCKASGY
TFTNYVMHWVRQAPGQ [symbol] LEWMGYINPYNDDVKYNEKFKGRVTITADESTST
AYMELSSLRSEDTAVYYCARWGYYGSPLYYFDYWGQGTLVTVSSASDKTHT
CPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKA
LPAPIEKTISKAKGQPREPRVYTLPP [symbol] RDELTKNQVSLTCLVKGFYPSDIA
VEWESNGQPENNYKTTPPVLVSDGSFTLYSKLTVDKSRWQQGNVFSCSVMH
EALHNHYTQKSLSLSPG

SEQ ID NO:331, as described above, represents the heavy chain portion of an Fab fragment derived from A49MI, which comprises a heavy chain variable domain (SEQ ID NO:351) of an NKG2D-binding site and a CH1 domain, connected to an Fc domain. The Fc domain in SEQ ID NO:331 includes a Y349C substitution in the CH3 domain, which forms a disulfide bond with the S354C substitution on the Fc linked to the B7-H3-binding scFv (SEQ ID NO:336). In SEQ ID NO:331, the Fe domain also includes K360E and K409W substitutions.

SEQ ID NO:332, as described above, represents the light chain portion of an Fab fragment derived from A49MI comprising a light chain variable domain (SEQ ID NO:86) of an NKG2D-binding site and a light chain constant domain.

In certain embodiments, a TriNKET of the present disclosure is identical to A49MI-F3′-TriNKET-huM30, except that the Fc domain linked to the NKG2D-binding Fab fragment comprises the substitutions of Q347R, D399V, and F405T, and the Fc domain linked to the huM30 scFv comprises matching substitutions K360E and K409W for forming a heterodimer. In certain embodiments, a TriNKET of the present disclosure is identical to A49MI-F3′-huM30, except that the Fc domain linked to the NKG2D-binding Fab fragment includes an S354C substitution in the CH3 domain, and the Fc domain linked to the huM30 scFv includes a matching Y349C substitution in the CH3 domain for forming a disulfide bond.

Another TriNKET of the present disclosure is A49MI-F3′-TriNKET-huAb13v1, sequences of which are provided below (CDRs (Chothia numbering) are underlined).

A49MI-F3′-TriNKET-huAb13v1 includes an scFv (SEQ ID NO:333) derived from huAb13v1 that binds B7-H3, linked to an Fc domain via a hinge comprising Ala-Ser (the sequence of the scFv-Fc polypeptide is represented by SEQ ID NO:334); and an NKG2D-binding Fab fragment derived from A49MI including a heavy chain portion comprising a heavy chain variable domain (SEQ ID N:351) and a CH1 domain, and a light chain portion comprising a light chain variable domain (SEQ ID NO:86) and a light chain constant domain, wherein the heavy chain variable domain is connected to the CH1 domain, and the CH1 domain is connected to an Fc domain that forms a dimer with the Fc domain that is linked to the B7-H3 binding scFv.

The B7-H3-binding scFv includes a heavy chain variable domain of huAb13v1 connected to a light chain variable domain of huAb13v1 with a (G₄S)₄ linker. The heavy and the light variable domains of the scFv (SEQ ID NO:333) are connected as VL-(G₄S)₄—VH; VL and VH contain 100VL-44VH S-S bridge as a result of G100C and G44C substitutions, respectively. In the amino acid sequences of SEQ ID NO:333 and SEQ ID N:334 below, the cysteine residues are bold-italics-underlined, and the (G₄S)₄ linker (GGGGSGGGGSGGGGSGGGGS, SEQ ID NO:126) is bold-underlined.

huAb13v1 scFv
(SEQ ID NO: 333)
DIQMTQSPSSLSASVGDRVTITCKASQNVGFNVAWYQQKPGKSPKALIYSA
SYRYSGVPSRFSGSGSGTDFTLTISSLQPEDFAEYFCQQYNWYPFTFG [symbol] GT
KLEIKGGGGSGGGGSGGGGSGGGGSEVQLQESGPGLVKPSETLSLTCAVTG
YSITSGYSWHWIRQFPGN [symbol] LEWMGYIHSSGSTNYNPSLKSRISISRDTSKN
QFFLKLSSVTAADTAVYYCAGYDDYFEYWGQGTTVTVSS

SEQ ID NO:334 represents the full sequence of a B7-H3-binding scFv linked to an Fe domain via a hinge comprising Ala-Ser (scFv-Fc). The Fe domain linked to the scFv includes Q347R, D399V, and F405T substitutions, which are bold-underlined in the sequence below. This Fe domain further includes an S354C substitution, which is bold-italics-underlined.

huAb13v1 scFv-Fc
(SEQ ID NO: 334)
DIQMTQSPSSLSASVGDRVTITCKASQNVGFNVAWYQQKPGKSPKALIYSA
SYRYSGVPSRFSGSGSGTDFTLTISSLQPEDFAEYFCQQYNWYPFTFG [symbol] GT
KLEIKGGGGSGGGGSGGGGSGGGGSEVQLQESGPGLVKPSETLSLTCAVTG
YSITSGYSWHWIRQFPGN [symbol] LEWMGYIHSSGSTNYNPSLKSRISISRDTSKN
QFFLKLSSVTAADTAVYYCAGYDDYFEYWGQGTTVTVSSASDKTHTCPPCP
APELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPI
EKTISKAKGQPREPRVYTLPP [symbol] RDELTKNQVSLTCLVKGFYPSDIAVEWES
NGQPENNYKTTPPVLVSDGSFTLYSKLTVDKSRWQQGNVFSCSVMHEALHN
HYTQKSLSLSPG

SEQ ID NO:331, as described above, represents the heavy chain portion of an Fab fragment derived from A49MI, which comprises a heavy chain variable domain (SEQ ID NO:351) of an NKG2D-binding site and a CH1 domain, connected to an Fc domain. The Fc domain in SEQ ID NO:331 includes a Y349C substitution in the CH3 domain, which forms a disulfide bond with the S354C substitution on the Fc linked to the B7-H3-binding scFv (SEQ ID NO:334). In SEQ ID NO:331, the Fc domain also includes K360E and K409W substitutions.

SEQ ID NO:332, as described above, represents the light chain portion of an Fab fragment comprising a light chain variable domain (SEQ ID NO:86) of an NKG2D-binding site and a light chain constant domain.

In certain embodiments, a TriNKET of the present disclosure is identical to A49MI-F3′-TriNKET-huAb13v1, except that the Fc domain linked to the NKG2D-binding Fab fragment comprises the substitutions of Q347R, D399V, and F405T, and the Fc domain linked to the huAb13v1 scFv comprises matching substitutions K360E and K409W for forming a heterodimer. In certain embodiments, a TriNKET of the present disclosure is identical to A49MI-F3′-TriNKET-huAb13v1, except that the Fc domain linked to the NKG2D-binding Fab fragment includes an S354C substitution in the CH3 domain, and the Fc domain linked to the huAb13v1 scFv includes a matching Y349C substitution in the CH3 domain for forming a disulfide bond.

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

The multi-specific proteins described herein contain an antigen-binding site that binds NKG2D-binding site, an antigen-binding site that binds B7-H3, and an antibody Fc domain or a portion thereof sufficient to bind CD16, or an antigen-binding site that binds CD16. In some embodiments, the multi-specific proteins contain an additional antigen-binding site that binds to B7-H3 as exemplified in the F4-TriNKET format.

In some embodiments, the multi-specific proteins display similar thermal stability to the corresponding B7-H3 monoclonal antibody, i.e., a monoclonal antibody containing the same B7-H3-binding site as the one incorporated in the multi-specific proteins.

In some embodiments, the multi-specific proteins bind to cells expressing NKG2D and/or CD16, such as NK cells, and tumor cells expressing B7-H3 simultaneously. Binding of the multi-specific proteins to NK cells can enhance the activity of the NK cells toward destruction of the tumor cells.

In some embodiments, the multi-specific proteins bind to B7-H3 with a similar affinity to the corresponding B7-H3 monoclonal antibody (i.e., a monoclonal antibody containing the same B7-H3-binding site as the one incorporated in the multi-specific proteins). In some embodiments, the multi-specific proteins are more effective in killing the tumor cells expressing B7-H3 than the corresponding B7-H3 monoclonal antibodies.

In some embodiments, the multi-specific proteins activate primary human NK cells when co-culturing with cells expressing B7-H3. NK cell activation is marked by the increase in CD107a degranulation and IFN-γ cytokine production. Furthermore, compared to the corresponding B7-H3 monoclonal antibody, the multi-specific proteins can show superior activation of human NK cells in the presence of cells expressing B7-H3.

In some embodiments, the multi-specific proteins enhance the activity of rested and IL-2-activated human NK cells when co-culturing with cells expressing B7-H3.

In some embodiments, compared to the corresponding monoclonal antibody that binds to B7-H3, the multi-specific proteins offer an advantage in targeting tumor cells that express medium and low levels of B7-H3.

In some embodiments, the bivalent F4 format of the TriNKETs (i.e., TriNKETs include an additional antigen-binding site that binds to B7-H3) improve the avidity with which the TriNKETs binds to B7-H3, which in effect stabilize expression and maintenance of high levels of B7-H3 on tumor cell surface. In some embodiments, the F4-TriNKETs mediate more potent killing of B7-H3-expressing tumor cells than the corresponding F3-TriNKETs or F3′-TriNKETs.

In some embodiments, the multi-specific proteins described herein contain an antigen-binding site that binds NKG2D-binding site, an antigen-binding site that binds L1CAM, and an antibody Fc domain or a portion thereof sufficient to bind CD16, or an antigen-binding site that binds CD16. In some embodiments, the multi-specific proteins contains an additional antigen-binding site that binds to L1CAM as exemplified in the F4-TriNKET format.

In some embodiments, the multi-specific proteins display similar thermal stability to the corresponding L1CAM monoclonal antibody, i.e., a monoclonal antibody containing the same L1CAM-binding site as the one incorporated in the multi-specific proteins.

In some embodiments, the multi-specific proteins bind to cells expressing NKG2D and/or CD16, such as NK cells, and tumor cells expressing L1CAM simultaneously. Binding of the multi-specific proteins to NK cells can enhance the activity of the NK cells toward destruction of the tumor cells.

In some embodiments, the multi-specific proteins bind to L1CAM with a similar affinity to the corresponding L1CAM monoclonal antibody (i.e., a monoclonal antibody containing the same L1CAM-binding site as the one incorporated in the multi-specific proteins). In some embodiments, the multi-specific proteins are more effective in killing the tumor cells expressing L1CAM than the corresponding L1CAM monoclonal antibodies.

In some embodiments, the multi-specific proteins activate primary human NK cells when co-culturing with cells expressing L1CAM. NK cell activation is marked by the increase in CD107a degranulation and IFN-γ cytokine production. Furthermore, compared to the corresponding L1CAM monoclonal antibody, the multi-specific proteins can show superior activation of human NK cells in the presence of cells expressing L1CAM.

In some embodiments, the multi-specific proteins enhance the activity of rested and IL-2-activated human NK cells when co-culturing with cells expressing L1CAM.

In some embodiments, compared to the corresponding monoclonal antibody that binds to L1CAM, the multi-specific proteins offer an advantage in targeting tumor cells that express medium and low levels of L1CAM.

In some embodiments, the bivalent F4 format of the TriNKETs (i.e., TriNKETs include an additional antigen-binding site that binds to L1CAM) improve the avidity with which the TriNKETs binds to L1CAM, which in effect stabilize expression and maintenance of high levels of L1CAM on tumor cell surface. In some embodiments, the F4-TriNKETs mediate more potent killing of L1CAM expressing tumor cells than the corresponding F3-TriNKETs or F3′-TriNKETs.

In some embodiments, the multi-specific proteins described herein include an NKG2D-binding site, a CD16-binding site, and an FLT1, KDR, TNC, TNN, CSPG4, BST1, SELP, CD200, INSR (HHF5), ITGA6, MELTF, PECAM1, or SLC1A5-binding site. In some embodiments, the multi-specific proteins bind to cells expressing NKG2D and/or CD16, such as NK cells, and tumor cells expressing FLT1, KDR, TNC, TNN, CSPG4, BST1, SELP, CD200, INSR (HHF5), ITGA6, MELTF, PECAM1, or SLC1A5 simultaneously. Binding of the multi-specific proteins to NK cells can enhance the activity of the NK cells toward destruction of the tumor cells.

In some embodiments, the multi-specific proteins bind to FLT1, KDR, TNC, TNN, CSPG4, BST1, SELP, CD200, INSR (HHF5), ITGA6, MELTF, PECAM1, or SLC1A5 with a similar affinity to the corresponding FLT1, KDR, TNC, TNN, CSPG4, BST1, SELP, CD200, INSR (HHF5), ITGA6, MELTF, PECAM1, or SLC1A5 monoclonal antibody (i.e., a monoclonal antibody containing the same antigen-binding site as the one incorporated in the multi-specific proteins). In some embodiments, the multi-specific proteins are more effective in killing the tumor cells expressing FLT1, KDR, TNC, TNN, CSPG4, BST1, SELP, CD200, INSR (HHF5), ITGA6, MELTF, PECAM1, or SLC1A5 than the corresponding FLT1, KDR, TNC, TNN, CSPG4, BST1, SELP, CD200, INSR (HHF5), ITGA6, MELTF, PECAM1, or SLC1A5 monoclonal antibodies.

In certain embodiments, the multi-specific proteins described herein, which include an NKG2D-binding site and a binding site for FLT1, KDR, TNC, TNN, CSPG4, BST1, SELP, CD200, INSR (HHF5), ITGA6, MELTF, PECAM1, or SLC1A5, activate primary human NK cells when co-culturing with cells expressing FLT1, KDR, TNC, TNN, CSPG4, BST1, SELP, CD200, INSR (HHF5), ITGA6, MELTF, PECAM1, or SLC1A5, respectively. NK cell activation is marked by the increase in CD107a degranulation and IFN-γ cytokine production. Furthermore, compared to a corresponding FLT1, KDR, TNC, TNN, CSPG4, BST1, SELP, CD200, INSR (HHF5), ITGA6, MELTF, PECAM1, or SLC1A5 monoclonal antibody, the multi-specific proteins may show superior activation of human NK cells in the presence of cells expressing FLT1, KDR, TNC, TNN, CSPG4, BST1, SELP, CD200, INSR (HHF5), ITGA6, MELTF, PECAM1, or SLC1A5, respectively.

In certain embodiments, the multi-specific proteins described herein, which include an NKG2D-binding site and a binding site for FLT1, KDR, TNC, TNN, CSPG4, BST1, SELP, CD200, INSR (HHF5), ITGA6, MELTF, PECAM1, or SLC1A5, enhance the activity of rested and TL-2-activated human NK cells co-culturing with cells expressing FLT1, KDR, TNC, TNN, CSPG4, BST1, SELP, CD200, INSR (HHF5), ITGA6, MELTF, PECAM1, or SLC1A5, respectively.

In certain embodiments, compared to a corresponding monoclonal antibody that binds to FLT1, KDR, TNC, TNN, CSPG4, BST1, SELP, CD200, INSR (HHF5), ITGA6, MELTF, PECAM1, or SLC1A5, the multi-specific proteins offer an advantage in targeting tumor cells that express medium and low levels of FLT1, KDR, TNC, TNN, CSPG4, BST1, SELP, CD200, INSR (HHF5), ITGA6, MELTF, PECAM1, or SLC1A5, respectively.

### III. Therapeutic Applications

The invention provides methods for treating cancer using a multi-specific binding protein described herein and/or a pharmaceutical composition described herein. The methods may be used to treat a variety of cancers expressing B7-H3. In some embodiments, the cancer is bladder cancer, breast cancer, cervical cancer, glioblastoma, head and neck cancer, lung cancer, liver cancer, melanoma, ovarian cancer, pancreatic cancer, prostate cancer, sarcoma, renal cancer, colorectal cancer, gastric cancer, neuroblastoma, squamous cell carcinoma, or acute myeloid leukemia (AML).

In some other embodiments, the cancer to be treated is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The invention provides methods for treating cancer using a multi-specific binding protein described herein and/or a pharmaceutical composition described herein. The methods may be used to treat a variety of cancers expressing L1CAM. In some embodiments, the cancer is bladder cancer, renal cancer, breast cancer, cervical cancer, sarcoma, lung cancer, head and neck cancer, glioblastoma, neuroblastoma, melanoma, ovarian cancer, endometrial cancer, esophageal cancer, gastric cancer, gastrointestinal stromal tumor (GIST), cholangiocarcinoma, colorectal cancer, pancreatic cancer, or prostate cancer.

The invention provides methods for treating cancer using a multi-specific binding protein described herein and/or a pharmaceutical composition described herein. In some embodiments, the invention provides methods for targeting cancers and/or neovasculature using a multi-specific binding protein described herein and/or a pharmaceutical composition described herein. The methods may be used to treat a variety of cancers expressing FLT1, KDR, TNC, TNN, CSPG4, BST1, SELP, CD200, INSR (HHF5), ITGA6, MELTF, PECAM1, or SLC1A5 (e.g., by tumor cells and/or by neovasculature).

In some embodiments, the method comprises administering to a patient in need thereof a FLT1-targeting multi-specific binding protein. Any FLT1-targeting multi-specific binding protein disclosed herein can be used in the method. Exemplary cancers to be treated by the FLT1-targeting multi-specific binding protein include but are not limited to renal cancer, gastric cancer, glioma, colorectal cancer, biliary tract cancer, prostate cancer, sarcoma, and breast cancer.

In some embodiments, the method comprises administering to a patient in need thereof a KDR-targeting multi-specific binding protein. Any KDR-targeting multi-specific binding protein disclosed herein can be used in the method. Exemplary cancers to be treated by the KDR-targeting multi-specific binding protein include but are not limited to renal cancer, gastric cancer, glioma, colorectal cancer, biliary tract cancer, lung cancer, melanoma, liver cancer, sarcoma, breast cancer, mesothelioma, and thyroid cancer.

In some embodiments, the method comprises administering to a patient in need thereof a TNC-targeting multi-specific binding protein. Any TNC-targeting multi-specific binding protein disclosed herein can be used in the method. Exemplary cancers to be treated by the TNC-targeting multi-specific binding protein include but are not limited to cervical cancer, breast cancer, pancreatic cancer, lung cancer, non-Hodgkin lymphoma, head and neck cancer, colorectal cancer, esophageal cancer, glioma, and prostate cancer.

In some embodiments, the method comprises administering to a patient in need thereof a TNN-targeting multi-specific binding protein. Any TNN-targeting multi-specific binding protein disclosed herein can be used in the method. Exemplary cancers to be treated by the TNN-targeting multi-specific binding protein include but are not limited to cervical cancer, breast cancer, pancreatic cancer, lung cancer, non-Hodgkin lymphoma, head and neck cancer, colorectal cancer, esophageal cancer, glioma, and prostate cancer.

In some embodiments, the method comprises administering to a patient in need thereof a CSPG4-targeting multi-specific binding protein. Any CSPG4-targeting multi-specific binding protein disclosed herein can be used in the method. Exemplary cancers to be treated by the CSPG4-targeting multi-specific binding protein include but are not limited to melanoma, renal cancer, sarcoma, glioma, head and neck cancer, breast cancer, bladder cancer, lung cancer, and cervical cancer.

In some embodiments, the method comprises administering to a patient in need thereof a BST1-targeting multi-specific binding protein. Any BST1-targeting multi-specific binding protein disclosed herein can be used in the method. Exemplary cancers to be treated by the BST1-targeting multi-specific binding protein include but are not limited to acute myeloid leukemia, mesothelioma, bladder cancer, and sarcoma.

In some embodiments, the method comprises administering to a patient in need thereof a SELP-targeting multi-specific binding protein. Any SELP-targeting multi-specific binding protein disclosed herein can be used in the method. Exemplary cancers to be treated by the SELP-targeting multi-specific binding protein include but are not limited to myeloproliferative neoplasms, acute myeloid leukemia, breast cancer, bladder cancer, thyroid cancer, renal cancer, and pancreatic cancer.

In some embodiments, the method comprises administering to a patient in need thereof a CD200-targeting multi-specific binding protein. Any CD200-targeting multi-specific binding protein disclosed herein can be used in the method. Exemplary cancers to be treated by the CD200-targeting multi-specific binding protein include but are not limited to breast cancer, colorectal cancer, B cell malignancies, multiple myeloma, acute myeloid leukemia, lymphoma, and mesothelioma.

In some embodiments, the method comprises administering to a patient in need thereof a INSR-targeting multi-specific binding protein. Any INSR-targeting multi-specific binding protein disclosed herein can be used in the method. Exemplary cancers to be treated by the INSR-targeting multi-specific binding protein include but are not limited to prostate cancer, gastric cancer, colorectal cancer, glioblastoma, breast cancer, prostate cancer, endometrial cancer, liver cancer, and renal cancer.

In some embodiments, the method comprises administering to a patient in need thereof a ITGA6-targeting multi-specific binding protein. Any ITGA6-targeting multi-specific binding protein disclosed herein can be used in the method. Exemplary cancers to be treated by the ITGA6-targeting multi-specific binding protein include but are not limited to breast cancer, leukemia, prostate cancer, colorectal cancer, renal cancer, head and neck cancer, ovarian cancer, gastric cancer, and lung cancer.

In some embodiments, the method comprises administering to a patient in need thereof a MELTF-targeting multi-specific binding protein. Any MELTF-targeting multi-specific binding protein disclosed herein can be used in the method. Exemplary cancers to be treated by the MELTF-targeting multi-specific binding protein include but are not limited to breast cancer, lung cancer, melanoma, bladder cancer, renal cancer, sarcoma, head and neck cancer, mesothelioma, pancreatic cancer.

In some embodiments, the method comprises administering to a patient in need thereof a PECAM1-targeting multi-specific binding protein. Any PECAM1-targeting multi-specific binding protein disclosed herein can be used in the method. Exemplary cancers to be treated by the PECAM1-targeting multi-specific binding protein include but are not limited to solid tumors. In some embodiments, these solid tumors are associated with significant neovasculature, for example, pancreatic cancer, prostate cancer, breast cancer, lung cancer, head and neck cancer, glioblastoma, and colorectal cancer.

In some embodiments, the method comprises administering to a patient in need thereof a SLC1A5-targeting multi-specific binding protein. Any SLC1A5-targeting multi-specific binding protein disclosed herein can be used in the method. Exemplary cancers to be treated by the SLC1A5-targeting multi-specific binding protein include but are not limited to lung cancer, colorectal cancer, breast cancer, prostate cancer, renal cancer, head and neck cancer, neuroblastoma, gastric cancer, and ovarian cancer.

In some embodiments, the cancer is a solid tumor. In some embodiments, the cancer is breast, ovarian, esophageal, bladder or gastric cancer, salivary duct carcinomas, adenocarcinoma of the lung or aggressive forms of uterine cancer, such as uterine serous endometrial carcinoma. In some other embodiments, the cancer is brain cancer, breast cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some embodiments, the cancer is leukemia, for example acute myeloid leukemia, T-cell leukemia, acute lymphocytic leukemia, chronic lymphocytic leukemia, chronic myeloid leukemia, or hairy cell leukemia. In some other embodiments, the cancer to be treated is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

### IV. Combination Therapy

Another aspect of the invention provides for combination therapy. A multi-specific binding protein described herein can be used in combination with additional therapeutic agents to treat cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H4, (vi) FLT1, KDR, TNC, TNN, CSPG4, BST1, SELP, CD200, INSR (HHF5), ITGA6, MELTF, PECAM1, or SLC1A5, (vii) B7-H3 and (viii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg—about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/mL of disodium phosphate dihydrate, 0.7 to 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10-14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of N₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The protein of the present disclosure could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 g to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 g/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 g to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1—NKG2D Binding Domains Bind to NKG2D

### NKG2D-Binding Domains Bind to Purified Recombinant NKG2D

The nucleic acid sequences of human, mouse or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D-binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D-binding domain clone, an isotype control or a positive control (comprising heavy chain and light chain variable domains selected from SEQ ID NOs:101-104, or anti-mouse NKG2D clones MI-6 and CX-5 available at eBioscience) was added to each well.

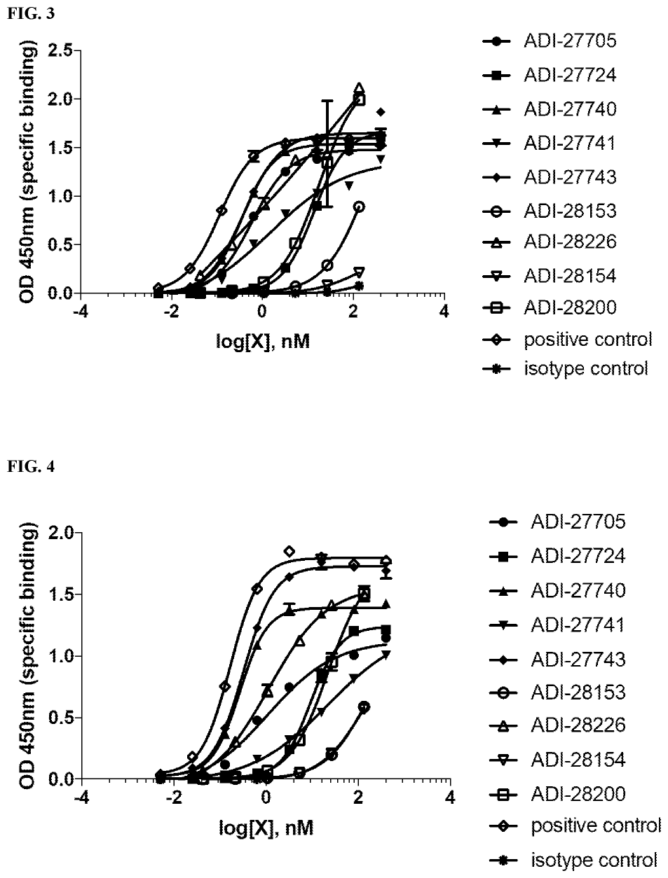

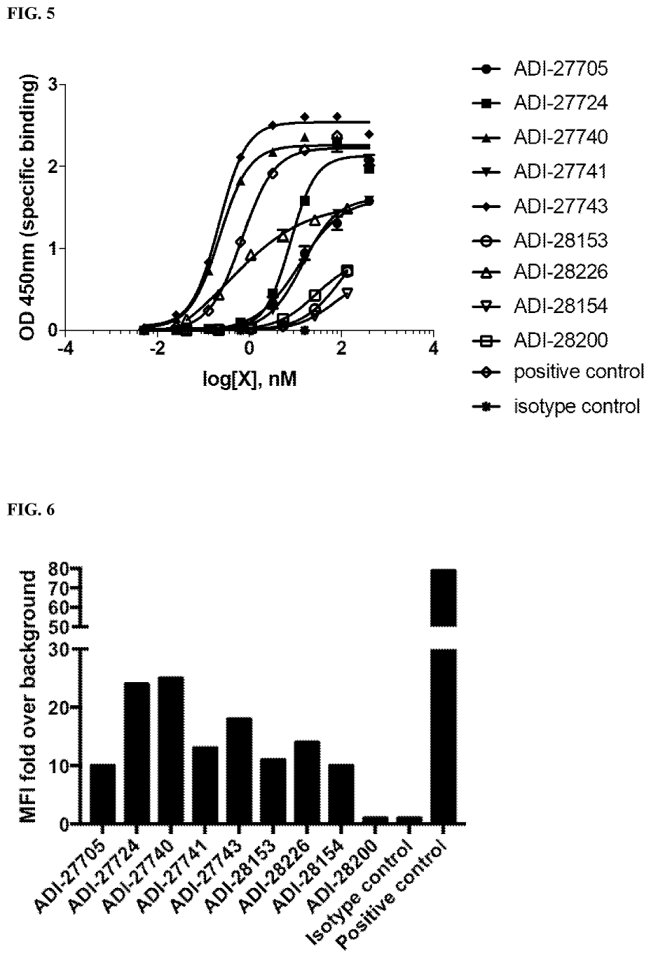

The isotype control showed minimal binding to recombinant NKG2D-Fc proteins, while the positive control bound strongest to the recombinant antigens. NKG2D-binding domains produced by all clones demonstrated binding across human, mouse, and cynomolgus recombinant NKG2D-Fc proteins, although with varying affinities from clone to clone. Generally, each anti-NKG2D clone bound to human (FIG. 3) and cynomolgus (FIG. 4) recombinant NKG2D-Fc with similar affinity, but with lower affinity to mouse (FIG. 5) recombinant NKG2D-Fc.

### NKG2D-Binding Domains Bind to Cells Expressing NKG2D

EL4 mouse lymphoma cell lines were engineered to express human or mouse NKG2D-CD3 zeta signaling domain chimeric antigen receptors. An NKG2D-binding clone, an isotype control or a positive control was used at a 100 nM concentration to stain extracellular NKG2D expressed on the EL4 cells. The antibody binding was detected using fluorophore-conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

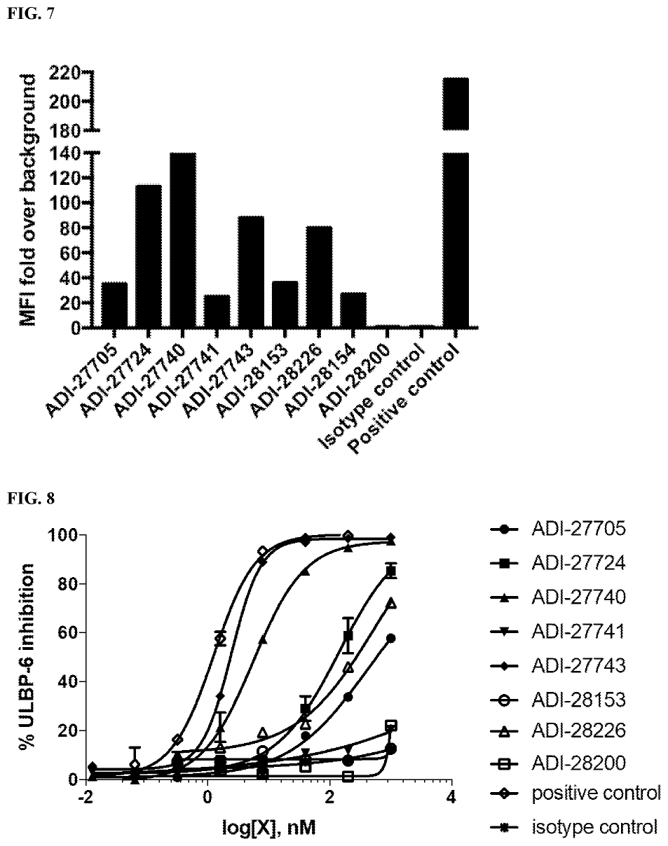

NKG2D-binding domains produced by all clones bound to EL4 cells expressing human and mouse NKG2D. Positive control antibodies (comprising heavy chain and light chain variable domains selected from SEQ ID NOs:101-104, or anti-mouse NKG2D clones MI-6 and CX-5 available at eBioscience) gave the best FOB binding signal. The NKG2D-binding affinity for each clone was similar between cells expressing human NKG2D (FIG. 6) and mouse (FIG. 7) NKG2D.

### Example 2—NKG2D-Binding Domains Block Natural Ligand Binding to NKG2D

Competition with ULBP-6

Recombinant human NKG2D-Fc proteins were adsorbed to wells of a microplate, and the wells were blocked with bovine serum albumin to reduce non-specific binding. A saturating concentration of ULBP-6-His-biotin was added to the wells, followed by addition of the NKG2D-binding domain clones. After a 2-hour incubation, wells were washed and ULBP-6-His-biotin that remained bound to the NKG2D-Fc coated wells was detected by streptavidin-conjugated to horseradish peroxidase and TMB substrate. Absorbance was measured at 450 nM and corrected at 540 nM. After subtracting background, specific binding of NKG2D-binding domains to the NKG2D-Fc proteins was calculated from the percentage of ULBP-6-His-biotin that was blocked from binding to the NKG2D-Fc proteins in wells. The positive control antibody (comprising heavy chain and light chain variable domains selected from SEQ ID NOs:101-104) and various NKG2D-binding domains blocked ULBP-6 binding to NKG2D, while isotype control showed little competition with ULBP-6 (FIG. 8).

ULBP-6 sequence is represented by SEQ ID NO:108.

(SEQ ID NO: 108)
MAAAAIPALLLCLPLLFLLFGWSRARRDDPHSLCYDITVIPKFRPGPRWCA
VQGQVDEKTFLHYDCGNKTVTPVSPLGKKLNVTMAWKAQNPVLREVVDILT
EQLLDIQLENYTPKEPLTLQARMSCEQKAEGHSSGSWQFSIDGQTFLLFDS
EKRMWTTVHPGARKMKEKWENDKDVAMSFHYISMGDCIGWLEDFLMGMDST
LEPSAGAPLAMSSGTTQLRATATTLILCCLLIILPCFILPGI

Competition with MICA

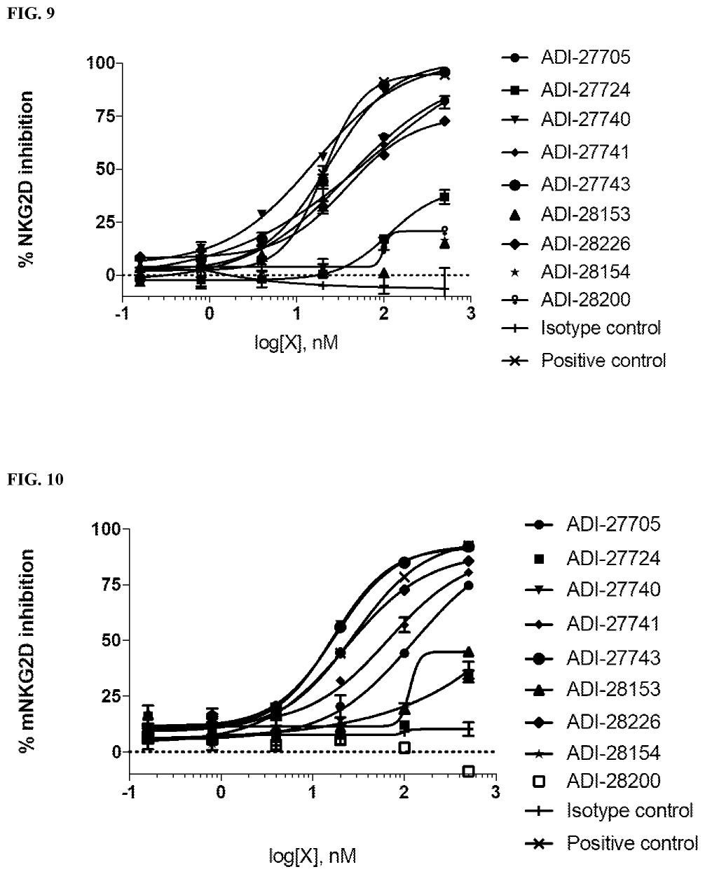

Recombinant human MICA-Fc proteins were adsorbed to wells of a microplate, and the wells were blocked with bovine serum albumin to reduce non-specific binding. NKG2D-Fc-biotin was added to wells followed by NKG2D-binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the MICA-Fc coated wells. The positive control antibody (comprising heavy chain and light chain variable domains selected from SEQ ID NOs:101-104) and various NKG2D-binding domains blocked MICA binding to NKG2D, while isotype control showed little competition with MICA (FIG. 9).

Competition with Rae-1 Delta

Recombinant mouse Rae-1delta-Fc (purchased from R&D Systems) was adsorbed to wells of a microplate, and the wells were blocked with bovine serum albumin to reduce non-specific binding. Mouse NKG2D-Fc-biotin was added to the wells followed by NKG2D-binding domains. After incubation and washing, NKG2D-Fc-biotin that remained bound to Rae-1delta-Fc coated wells was detected using streptavidin-RP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the Rae-1delta-Fc coated wells. The positive control (comprising heavy chain and light chain variable domains selected from SEQ ID NOs:101-104, or anti-mouse NKG2D clones MI-6 and CX-5 available at eBioscience) and various NKG2D-binding domain clones blocked Rae-1delta binding to mouse NKG2D, while the isotype control antibody showed little competition with Rae-1delta (FIG. 10).

### Example 3—NKG2D-Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

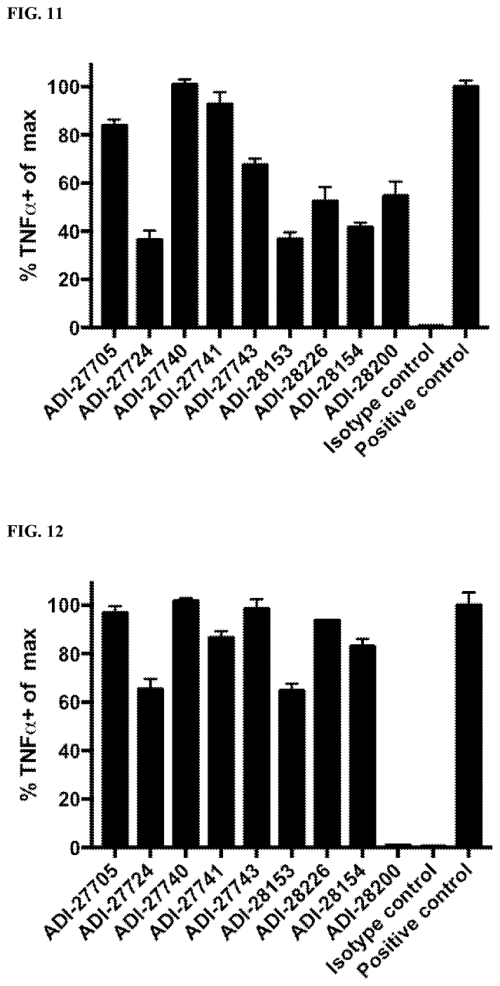

To determine whether NKG2D-binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-α production, an indicator for NKG2D activation, was assayed by flow cytometry. The percentage of TNF-α positive cells was normalized to the cells treated with the positive control. All NKG2D-binding domains activated both human NKG2D (FIG. 11) and mouse NKG2D (FIG. 12).

### Example 4—NKG2D-Binding Domains Activate NK Cells

### Primary Human NK Cells

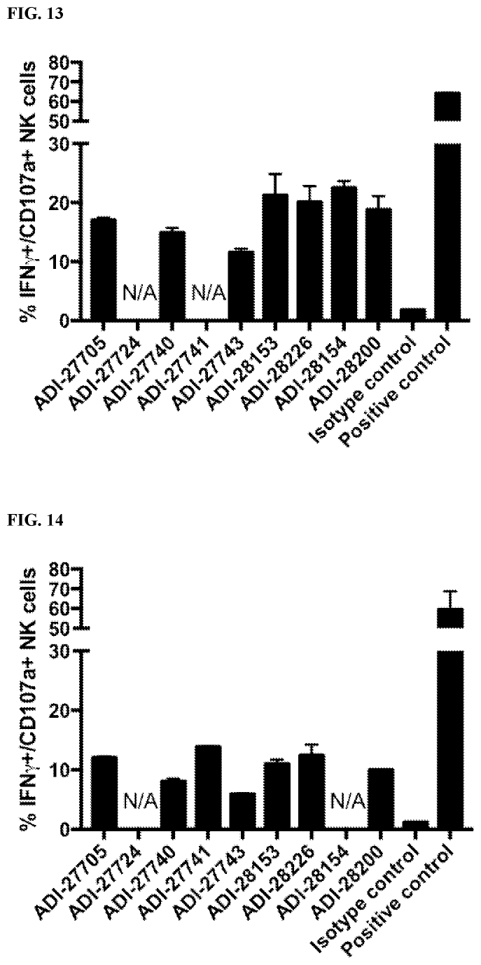

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL TL-2 for 24-48 hours before they were transferred to the wells of a microplate to which the NKG2D-binding domains were adsorbed, and cultured in the media containing fluorophore-conjugated anti-CD107a antibody, brefeldin-A, and monensin. Following culture, NK cells were assayed by flow cytometry using fluorophore-conjugated antibodies against CD3, CD56 and IFN-γ. CD107a and IFN-γ staining were analyzed in CD3⁻CD56⁺ cells to assess NK cell activation. The increase in CD107a/IFN-γ double-positive cells is indicative of better NK cell activation through engagement of two activating receptors rather than one receptor. NKG2D-binding domains and the positive control (e.g., heavy chain variable domain represent by SEQ ID NO:101 or SEQ ID NO:103, and light chain variable domain represented by SEQ ID NO:102 or SEQ ID NO:104) showed a higher percentage of NK cells becoming CD107a⁺ and IFN-γ⁺ than the isotype control (FIG. 13 & FIG. 14 represent data from two independent experiments, each using a different donor's PBMC for NK cell preparation).

### Primary Mouse NK Cells

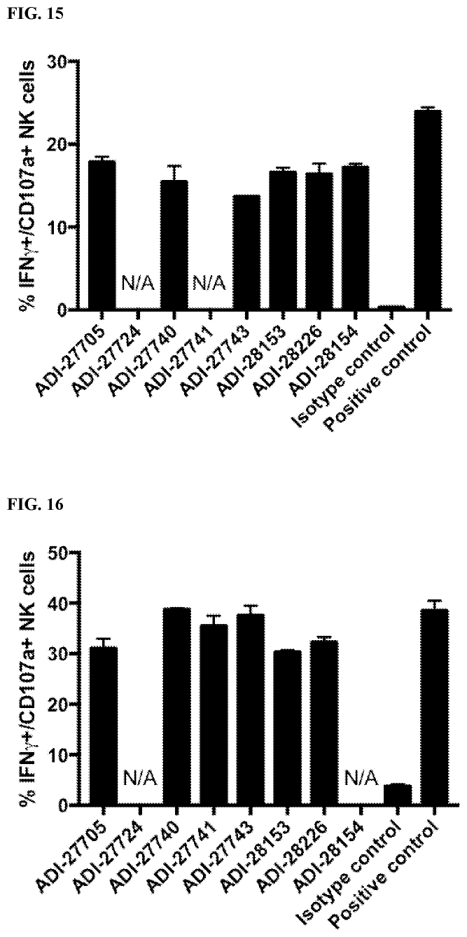

Spleens were obtained from C57B1/6 mice and crushed through a 70 μm cell strainer to obtain single cell suspension. Cells were pelleted and resuspended in ACK lysis buffer (purchased from Thermo Fisher Scientific #A1049201; 155 mM ammonium chloride, 10 mM potassium bicarbonate, 0.01 mM EDTA) to remove red blood cells. The remaining cells were cultured with 100 ng/mL hL-2 for 72 hours before being harvested and prepared for NK cell isolation. NK cells (CD3⁻NK1.1⁺) were then isolated from spleen cells using a negative depletion technique with magnetic beads with typically >90% purity. Purified NK cells were cultured in media containing 100 ng/mL mIL-15 for 48 hours before they were transferred to the wells of a microplate to which the NKG2D-binding domains were adsorbed, and cultured in the media containing fluorophore-conjugated anti-CD107a antibody, brefeldin-A, and monensin. Following culture in NKG2D-binding domain-coated wells, NK cells were assayed by flow cytometry using fluorophore-conjugated antibodies against CD3, NK1.1 and IFN-γ. CD107a and IFN-γ staining were analyzed in CD3⁻NK1.1⁺ cells to assess NK cell activation. The increase in CD107a/IFN-γ double-positive cells is indicative of better NK cell activation through engagement of two activating receptors rather than one receptor. NKG2D-binding domains and the positive control (selected from anti-mouse NKG2D clones MI-6 and CX-5 available at eBioscience) showed a higher percentage of NK cells becoming CD107a⁺ and IFN-γ⁺ than the isotype control (FIG. 15 & FIG. 16 represent data from two independent experiments, each using a different mouse for NK cell preparation).

### Example 5—NKG2D-Binding Domains Enable Cytotoxicity of Target Tumor Cells

Human and mouse primary NK cell activation assays demonstrated increased cytotoxicity markers on NK cells after incubation with NKG2D-binding domains. To address whether this translates into increased tumor cell lysis, a cell-based assay was utilized where each NKG2D-binding domain was developed into a monospecific antibody. The Fc region was used as one targeting arm, while the Fab fragment (NKG2D-binding domain) acted as another targeting arm to activate NK cells. THP-1 cells, which are of human origin and express high levels of Fc receptors, were used as a tumor target and a Perkin Elmer DELFIA Cytotoxicity Kit was used. THP-1 cells were labeled with BATDA reagent, and resuspended at 10⁵/mL in culture media. Labeled THP-1 cells were then combined with NKG2D antibodies and isolated mouse NK cells in wells of a microtiter plate at 37° C. for 3 hours. After incubation, 20 μL of the culture supernatant were removed, mixed with 200 μL of Europium solution and incubated with shaking for 15 minutes in the dark. Fluorescence was measured over time by a PheraStar plate reader equipped with a time-resolved fluorescence module (Excitation 337 nm, Emission 620 nm) and specific lysis was calculated according to the kit instructions.

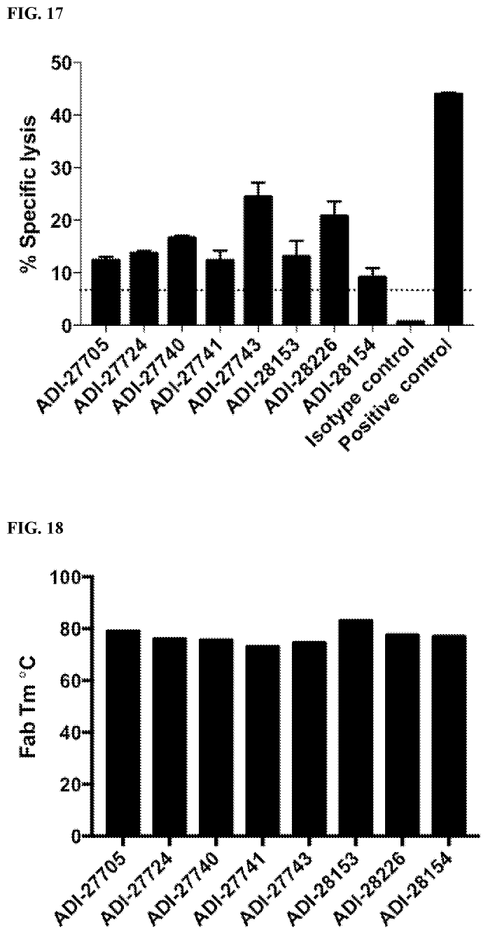

The positive control, ULBP-6—a natural ligand for NKG2D, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 17).

### Example 6—NKG2D Antibodies Show High Thermostability

Melting temperatures of NKG2D-binding domains were assayed using differential scanning fluorimetry. The extrapolated apparent melting temperatures are high relative to typical IgG1 antibodies (FIG. 18).

### Example 7—Synergistic Activation of Human NK Cells by Cross-Linking NKG2D and CD16

### Primary Human NK Cell Activation Assay

Peripheral blood mononuclear cells (PBMCs) were isolated from peripheral human blood buffy coats using density gradient centrifugation. NK cells were purified from PBMCs using negative magnetic beads (StemCell #17955). NK cells were >90% CD3⁻CD56⁺ as determined by flow cytometry. Cells were then expanded 48 hours in media containing 100 ng/mL hIL-2 (Peprotech #200-02) before use in activation assays. Antibodies were coated onto a 96-well flat-bottom plate at a concentration of 2 μg/mL (anti-CD16, Biolegend #302013) and 5 μg/mL (anti-NKG2D, R&D #MAB139) in 100 μL sterile PBS overnight at 4° C. followed by washing the wells thoroughly to remove excess antibody. For the assessment of degranulation IL-2-activated NK cells were resuspended at 5×10⁵ cells/mL in culture media supplemented with 100 ng/mL human IL-2 (hIL2) and 1 μg/mL APC-conjugated anti-CD107a mAb (Biolegend #328619). 1×10⁵ cells/well were then added onto antibody coated plates. The protein transport inhibitors Brefeldin A (BFA, Biolegend #420601) and Monensin (Biolegend #420701) were added at a final dilution of 1:1000 and 1:270, respectively. Plated cells were incubated for 4 hours at 37° C. in 5% CO₂. For intracellular staining of IFN-γ, NK cells were labeled with anti-CD3 (Biolegend #300452) and anti-CD56 mAb (Biolegend #318328), and subsequently fixed, permeabilized and labeled with anti-IFN-γ mAb (Biolegend #506507). NK cells were analyzed for expression of CD107a and IFN-γ by flow cytometry after gating on live CD56⁺CD3⁻ cells.

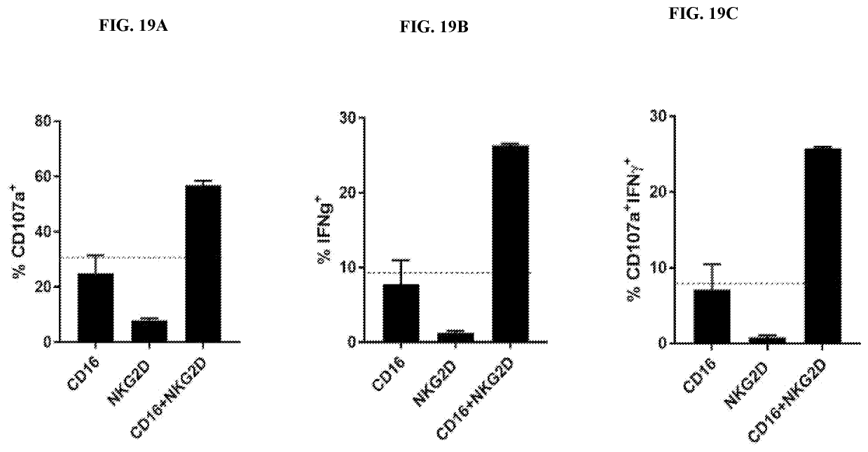

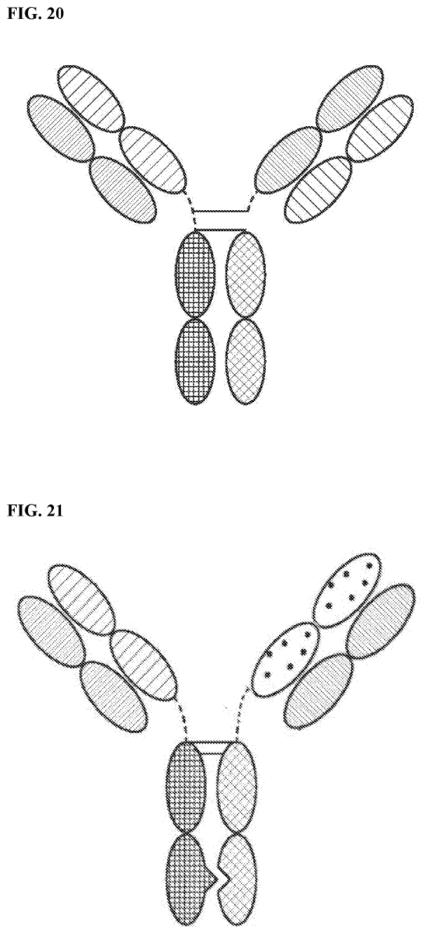

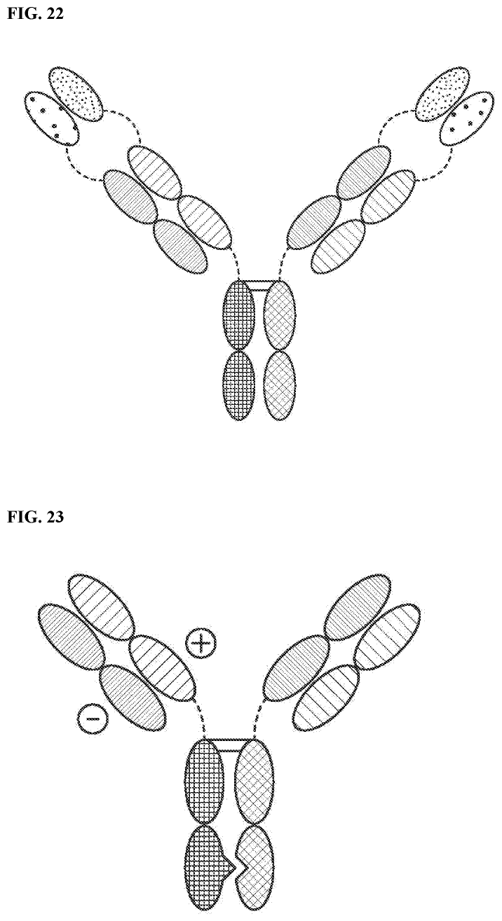

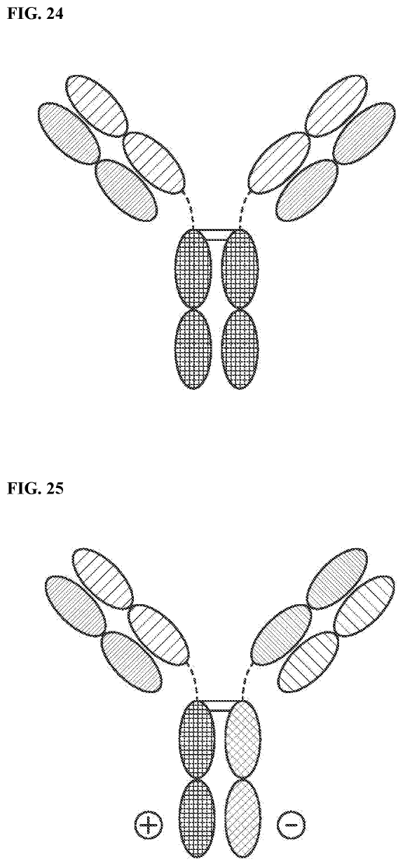

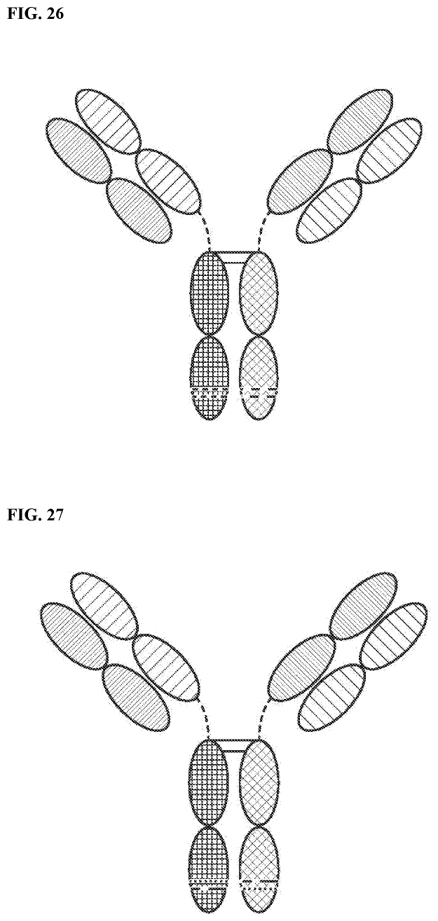

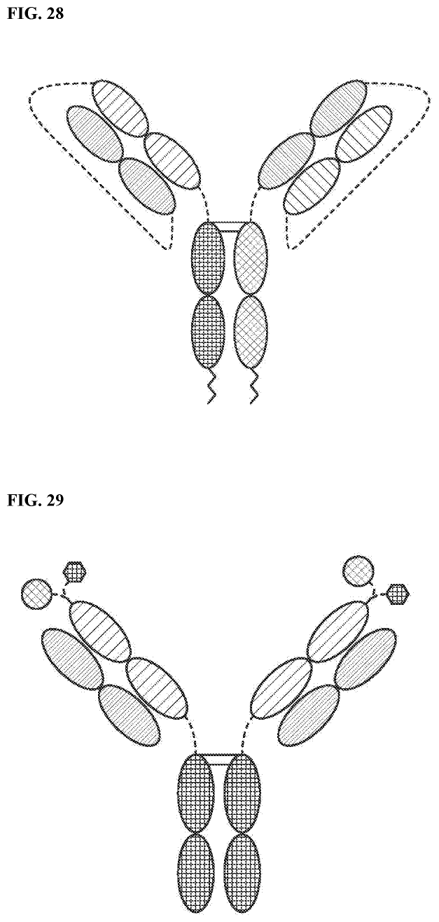

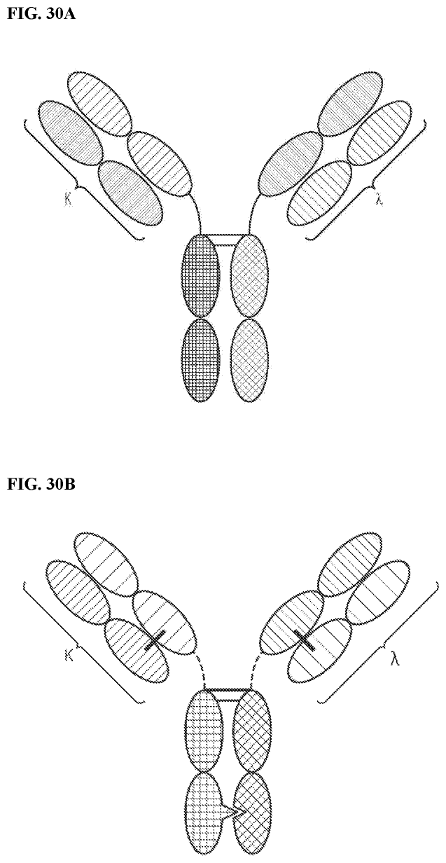

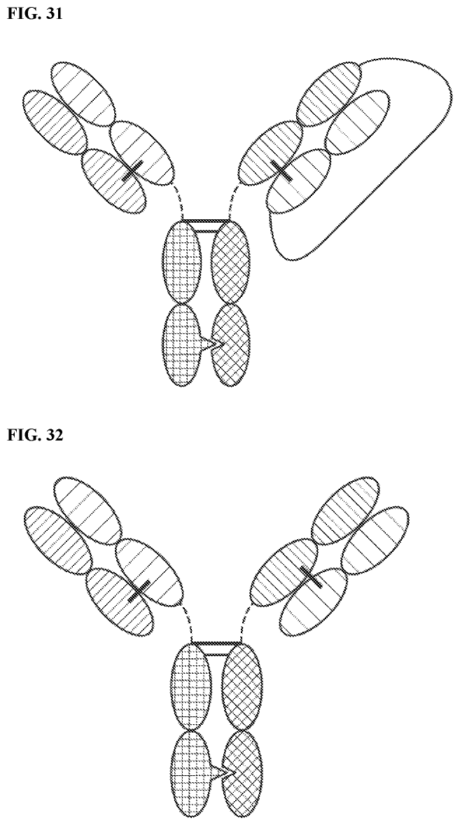

To investigate the relative potency of receptor combination, crosslinking of NKG2D or CD16, and co-crosslinking of both receptors by plate-bound stimulation was performed. As shown in FIG. 19 (FIGS. 19A-19C), combined stimulation of CD16 and NKG2D resulted in highly elevated levels of CD107a (degranulation) (FIG. 19A) and/or IFN-γ production (FIG. 19B). Dotted lines represent an additive effect of individual stimulations of each receptor.

CD107a levels and intracellular IFN-γ production of IL-2-activated NK cells were analyzed after 4 hours of plate-bound stimulation with anti-CD16, anti-NKG2D or a combination of both monoclonal antibodies. Graphs indicate the mean (n=2) SD. FIG. 19A demonstrates levels of CD107a; FIG. 19B demonstrates levels of IFN-γ; FIG. 19C demonstrates levels of CD107a and IFN-γ. Data shown in FIGS. 19A-19C are representative of five independent experiments using five different healthy donors.

### Example 8—Assessment of TriNKET or mAb Binding to Cell Expressed Human Cancer Antigens

Human cancer cell lines expressing B7-H3 were used to assess tumor antigen binding of TriNKETs derived from B7-H3 targeting clones. Human breast cancer cell lines BT-474 and HCC1954 and renal cancer cell line 786-O were used to assess binding of TriNKETs to cell expressed B7-H3. Varying concentrations of either TriNKET or monoclonal antibody were allowed to bind cells for 20 minutes on ice, after which cells were washed and the amount of bound TriNKET/monoclonal antibody was measured using fluorophore conjugated anti-human IgG secondary antibody. Cells were then analyzed by flow cytometry and binding MFI to cell expressed B7-H3 was plotted against varying concentration.

FIG. 35 shows binding of B7-H3-targeted TriNKETs and their parental monoclonal antibodies to B7-H3-expressing human cancer cell lines (A) 786-O, (B) BT-474 and (C) HCC1954. Three different B7-H3 binding domains were converted to single-chain variable fragments and expressed as TriNKETs with the same NKG2D binding domain. TriNKETs bearing 13v1, M30 and Enoblituzumab B7-H3 targeting domains showed positive binding to B7-H3-expressing human cancer cell lines. However, on all three lines, B7-H3 TriNKETs bind more weakly compared to their parental monoclonal antibodies. Reduced binding affinity can be attributed to conversion from Fab to scFv and/or monovalent binding of F3′ TriNKETs to B7-H3 compared to parental mAbs, which has two B7-H3 binding domains per molecule.

### Example 9—Primary Human NK Cell Cytotoxicity Assay

PBMCs were isolated from human peripheral blood buffy coats using density gradient centrifugation, washed, and prepared for NK cell isolation. NK cells were isolated using a negative selection technique with magnetic beads. Purity of isolated NK cells was typically >90% CD3−CD56+. Isolated NK cells were rested overnight and used the following day in cytotoxicity assays.

### DELFIA Cytotoxicity Assay

Human cancer cell lines expressing a target of interest, B7-H3, were harvested from culture, cells were washed with HBS, and resuspended in growth media at 10⁶/mL for labeling with BATDA reagent (Perkin Elmer C136-100). Manufacturer instructions were followed for labeling of the target cells. After labeling, cells were washed 3× with HBS, and were resuspended at 0.5−1.0×10⁵/mL in culture media. To prepare the background wells an aliquot of the labeled cells was put aside, and the cells were spun out of the media. 100 μl of media were carefully added to wells in triplicate to avoid disturbing the pelleted cells. 100 μl of BATDA labeled cells were added to each well of the 96-well plate. Wells were saved for spontaneous release from target cells prepared for maximum lysis of target cells adding 1% Triton-X. Monoclonal antibodies or TriNKETs against B7-H3 were diluted in culture media and 50 μl of diluted mAb or TriNKET were added to each well. Rested and/or activated NK cells were harvested from culture; cells were then washed and resuspended at concentrations of 10⁵−2.0×10⁶/mL in culture media for an E:T ratio of 5:1. 50 μl of NK cells were added to each well of the plate for a total of 200 μl culture volume. The plate was incubated at 37° C. with 5% CO₂ for 2-3 hours before developing the assay.

After culturing for 2-3 hours, the plate was removed from the incubator and the cells were pelleted by centrifugation at 200×g for 5 minutes. 20 μl of culture supernatant were transferred to a clean microplate and 200 μl of room temperature europium solution (Perkin Elmer C135-100) were added to each well. The plate was protected from light and incubated on a plate shaker at 250 rpm for 15 minutes, then read using SpectraMax i3X instruments. % Specific lysis was calculated as follows:

% Specific lysis=((Experimental release−Spontaneous release)/(Maximum release−Spontaneous release))*100%.

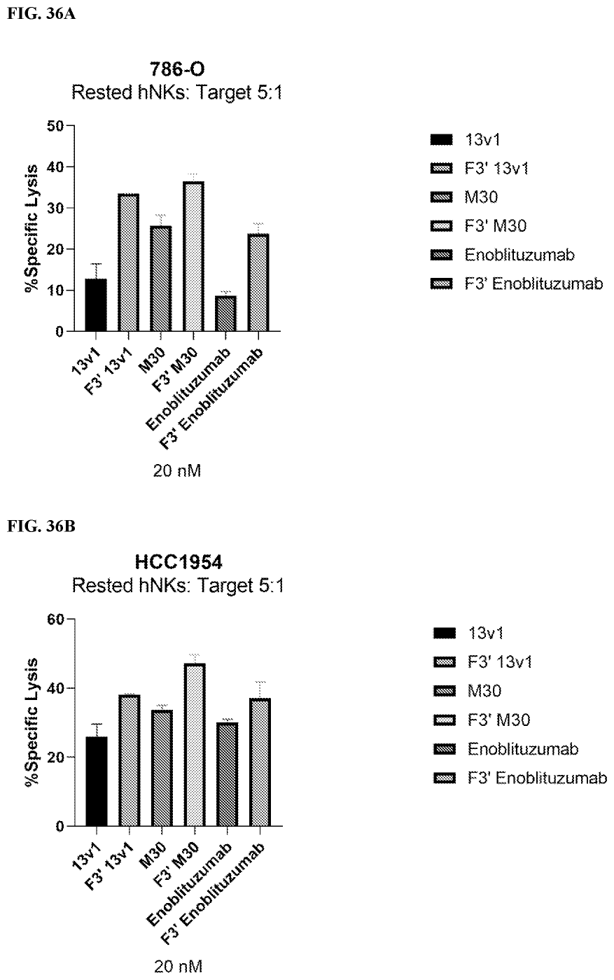

FIG. 36 shows the activity of 20 nM B7-H3 targeted TriNKET or parental mAb in enhancing primary NK cell-mediated killing of 786-O (FIG. 36A) and HCC1954 (FIG. 36B) cancer cell lines. Despite TriNKETs having weakened binding to B7-H3-expressing cancer cells compared to parental monoclonal antibodies, TriNKETs enhance NK cell-mediated lysis of 786-O and HCC1954 cancer cells better than parental mAbs.

### Example 10—KHYG-1 CD16V Cell Cytotoxicity Assay

KHYG-1 cells are a highly cytotoxic NK leukemia cell line obtained from DSMZ (DSMZ #ACC725). Parental KHYG-1 cells express NKG2D but not CD16 on their cell surface. KHYG-1 cells transduced with high affinity human CD16 were used for a cell cytotoxicity assay. KHYG-1 CD16V cells were rested overnight and used the following day in cytotoxicity assays as effector cells.

### DELFIA Cytotoxicity Assay

Human cancer cell lines expressing B7-H3 were harvested from culture, washed with HBS, and resuspended in growth media at 10⁶/mL for labeling with BATDA reagent (Perkin Elmer C136-100) in accordance with the manufacturer's instructions. After labeling, cells were washed 3× with HBS and resuspended at 0.5−1.0×10⁵/mL in culture media. To prepare the background wells, an aliquot of the labeled cells was put aside, and the cells were spun out of the media. 100 μl of the media were carefully added to wells in triplicate to avoid disturbing the pelleted cells. 100 μl of BATDA labeled cells were added to each well of the 96-well plate. Wells were saved for spontaneous release from target cells, and prepared for maximum lysis of target cells by adding 1% Triton-X. Monoclonal antibodies or TriNKETs against B7-H3 were diluted in culture media and 50 μl of diluted mAb or TriNKET were added to each well. Rested KHYG-1 CD16V cells were harvested from culture, washed, and resuspended at 10⁵−2.0×10⁶/mL in culture media for an E:T ratio of 10:1. 50 μl of KHYG-1 CD16V cells were added to each well of the plate to make a total of 200 μl culture volume. The plate was incubated at 37° C. with 5% CO₂ for 2-3 hours before developing the assay.

After culturing for 2-3 hours, the plate was removed from the incubator and the cells were pelleted by centrifugation at 200×g for 5 minutes. 20 μl of culture supernatant were transferred to a clean microplate, 200 μl of room temperature europium solution (Perkin Elmer C135-100) were added to each well. The plate was protected from light and incubated on a plate shaker at 250 rpm for 15 minutes, then read using SpectraMax i3X instruments. Specific lysis was calculated as follows:

% Specific lysis=((Experimental release−Spontaneous release)/(Maximum release−Spontaneous release))*100%.

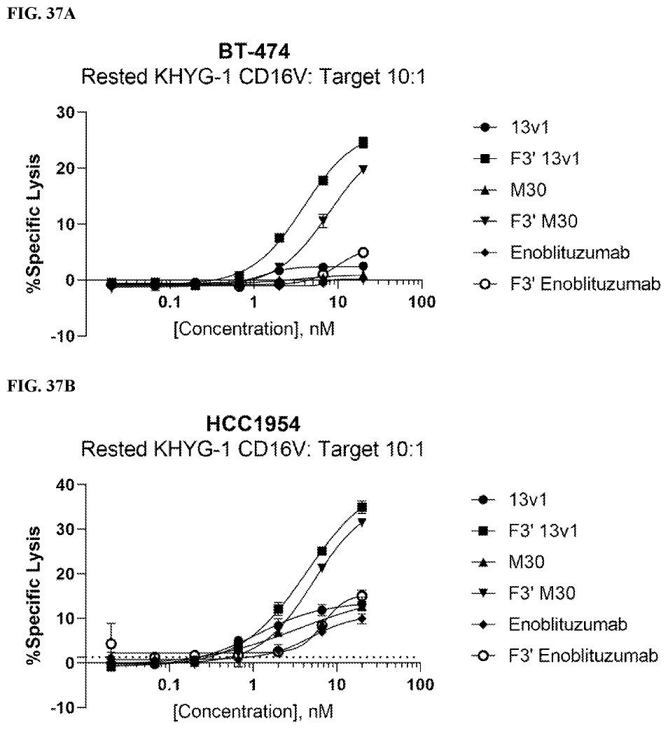

FIG. 37A and FIG. 37B show that B7-H3 targeted TriNKETs significantly enhance KHYG-1-CD16V cell killing of the BT-474 and HCC1954 cancer cell lines, respectively. TriNKETs are more potent with lower EC₅₀ values and reach higher maximum lysis than parental mAbs.

### Example 11—Co-Culture Activation Assay

PBMCs were isolated from human peripheral blood buffy coats using density gradient centrifugation. Isolated PBMCs were washed and rested overnight at 1×10⁶/mL n primary culture media. Human cancer cell lines expressing B7-H3 were harvested from culture, and cells were adjusted to 2×10⁶/mL. B7-H3 TriNKETs, B7-H3 parental monoclonal antibodies, or hIgG1 isotype control were diluted in culture media. Rested PBMCs were harvested from culture, washed, and resuspended at 4×10⁶/mL in culture media. IL-2 and fluorophore conjugated anti-CD107a were added to the PBMCs for the activation culture. Brefeldin-A and monensin were diluted into culture media to block protein transport out of the cell for intracellular cytokine staining. 50 μl of tumor targets, mAbs/TriNKETs, BFA/monensin, and PBMCs were added in a 96-well plate for a total culture volume of 200 μl. The plate was cultured for 4 hours before samples were prepared for FACS analysis.

Following the 4-hour activation culture, cells were prepared for analysis by flow cytometry using fluorophore conjugated antibodies against CD3, CD56 and IFNγ (Table 13). CD107a and IFNγ staining was analyzed in CD3−CD56+ populations to assess NK cell activation.

TABLE 13

| Channel | FITC | PE | PerCP | APC | APC-Cy7 | 421 |
|---|---|---|---|---|---|---|
| Marker | CD3 | IFNγ | CD45 | CD107a | L/D | CD56 |

Cells of interest were identified using an FSC vs. SSC plot and an appropriately shaped gate was drawn around the cells. Within the gated cells, doublet cells were removed by viewing the FSC-H vs. FSC-A plot. Within the single cell population, live cells were gated. Within live cells, NK cells were identified as CD56+CD3⁻. CD107a degranulation and IC IFNγ were analyzed within the NK cell population.

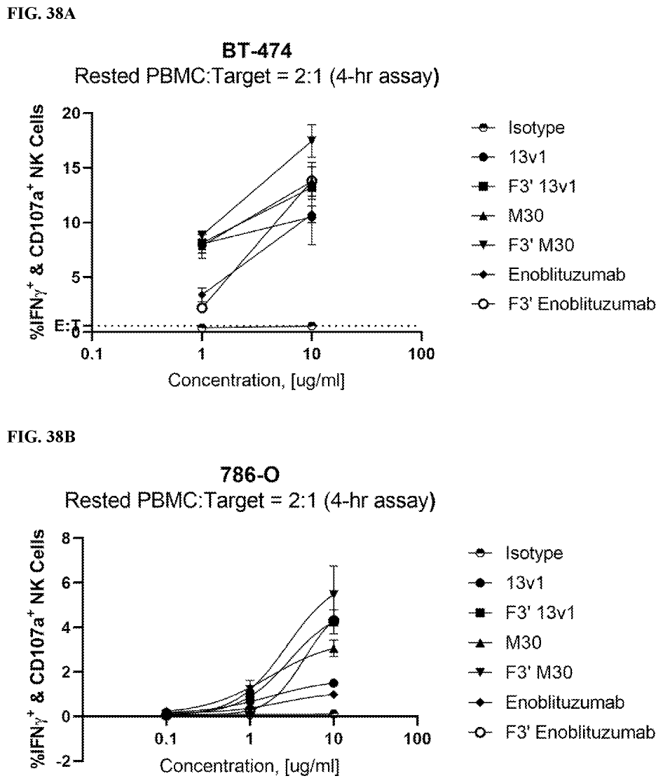

PBMCs were co-cultured with BT-474 and 786-O cells in the presence of B7-H3 TriNKET, monoclonal antibody or isotype hIgG1 isotype control. FIG. 38A and FIG. 38B show the percentage of NK cells expressing both IFNγ and CD107a after co-culture with B7-H3-expressing cancer cells BT-474 and 786-O, respectively. All B7-H3 TriNKETs and parental monoclonal antibodies induced intracellular IFNγ and CD107a degranulation in human NK cells. While isotype control treatment did not activate NK cells at all, the percentage of IFNγ and CD107a double-positive NK cells were higher in co-cultures treated with 10 μg/mL of B7-H3 TriNKETs compared to their respective parental mAbs, indicating that TriNKETs stimulate NK cells better than their parental mAbs.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A protein comprising:
(a) a first antigen-binding site comprising an Fab fragment that binds NKG2D;
(b) a second antigen-binding site comprising a single-chain variable fragment (scFv) that binds B7-H3; and
(c) an antibody Fe domain or a portion thereof sufficient to bind CD16, or a third antigen-binding site that binds CD16.

2. The protein of claim 1, wherein the scFv is linked to the antibody Fc domain or a portion thereof sufficient to bind CD16, or the third antigen-binding site that binds CD16, via a hinge comprising Ala-Ser or Gly-Ala-Ser, wherein the scFv comprises a heavy chain variable domain and a light chain variable domain.

3. The protein according to claim 2, wherein the scFv is linked to the antibody Fc domain.

4. The protein according to claim 2 or 3, wherein the heavy chain variable domain of the scFv forms a disulfide bridge with the light chain variable domain of the scFv.

5. The protein according to claim 4, wherein the disulfide bridge is formed between C44 from the heavy chain variable domain and C100 from the light chain variable domain.

6. The protein according to claim 5, wherein the scFv is linked to the antibody Fc domain, wherein the light chain variable domain of the scFv is positioned at the N-terminus of the heavy chain variable domain of the scFv, and is linked to the heavy chain variable domain of the scFv via a flexible linker (GyGyGlyGlySer)₄ ((G4S)₄) (SEQ ID NO:126), and the Fab is linked to the antibody Fc domain.

7. The protein according to any one of claims 2-6, wherein the heavy chain variable domain of the scFv is linked to the light chain variable domain of the scFv via a flexible linker.

8. The protein according to claim 7, wherein the flexible linker comprises (GlyGlyGlyGlySer)₄ ((G4S)₄) (SEQ ID NO:126).

9. The protein according to any one of claims 2-8, wherein the heavy chain variable domain of the scFv is positioned at the N-terminus or the C-terminus of the light chain variable domain of the scFv.

10. The protein according to claim 9, wherein the light chain variable domain of the scFv is positioned at the N-terminus of the heavy chain variable domain of the scFv.

11. The protein according to any one of claims 1 to 10, wherein the Fab fragment is linked to the antibody Fc domain or a portion thereof sufficient to bind CD16 or the third antigen-binding site that binds CD16.

12. The protein according claim 11, wherein the heavy chain portion of the Fab fragment comprises a heavy chain variable domain and a CH1 domain, and wherein the heavy chain variable domain is linked to the CH1 domain.

13. The protein according claim 11 or 12, wherein the Fab is linked to the antibody Fc domain.

14. A protein comprising:
(a) a first antigen-binding site that binds NKG2D;
(b) a second antigen-binding site that binds a tumor-associated antigen B7-H3; and
(c) an antibody Fc domain or a portion thereof sufficient to bind CD16, or a third antigen-binding site that binds CD16.

15. The protein according any one of claims 1-14, wherein the first antigen-binding site that binds NKG2D comprises:
(1) a heavy chain variable domain comprising complementarity-determining region 1 (CDR1), complementarity-determining region 2 (CDR2), and complementarity-determining region 3 (CDR3) sequences represented by the amino acid sequences of SEQ ID NOs: 347, 88, and 352, respectively; and a light chain variable domain comprising CDR1, CDR2, and CDR3 sequences represented by the amino acid sequences of SEQ ID NOs: 90, 91, and 92, respectively;
(2) a heavy chain variable domain comprising CDR1, CDR2, and CDR3 sequences represented by the amino acid sequences of SEQ ID NOs: 347, 88, and 348, respectively; and a light chain variable domain comprising CDR1, CDR2, and CDR3 sequences represented by the amino acid sequences of SEQ ID NOs: 90, 91, and 92, respectively;
(3) a heavy chain variable domain comprising CDR1, CDR2, and CDR3 sequences represented by the amino acid sequences of SEQ ID NOs: 341, 64, and 342, respectively; and a light chain variable domain comprising CDR1, CDR2, and CDR3 sequences represented by the amino acid sequences of SEQ ID NOs: 66, 67, and 68, respectively;
(4) a heavy chain variable domain comprising CDR1, CDR2, and CDR3 sequences represented by the amino acid sequences of SEQ ID NOs: 343, 72, and 344, respectively; and a light chain variable domain comprising CDR1, CDR2, and CDR3 sequences represented by the amino acid sequences of SEQ ID NOs: 74, 75, and 76, respectively;
(5) a heavy chain variable domain comprising CDR1, CDR2, and CDR3 sequences represented by the amino acid sequences of SEQ ID NOs: 345, 80, and 346, respectively; and a light chain variable domain comprising CDR1, CDR2, and CDR3 sequences represented by the amino acid sequences of SEQ ID NOs: 82, 83, and 84, respectively;
(6) a heavy chain variable domain comprising CDR1, CDR2, and CDR3 sequences represented by the amino acid sequences of SEQ ID NOs: 87, 88, and 89, respectively; and a light chain variable domain comprising CDR1, CDR2, and CDR3 sequences represented by the amino acid sequences of SEQ ID NOs: 90, 91, and 92, respectively;
(7) a heavy chain variable domain comprising CDR1, CDR2, and CDR3 sequences represented by the amino acid sequences of SEQ ID NOs: 349, 96, and 350, respectively; and a light chain variable domain comprising CDR1, CDR2, and CDR3 sequences represented by the amino acid sequences of SEQ ID NOs: 98, 99, and 100, respectively;
(8) a heavy chain variable domain comprising CDR1, CDR2, and CDR3 sequences represented by the amino acid sequences of SEQ ID NOs: 347, 88, and 355, respectively; and a light chain variable domain comprising CDR1, CDR2, and CDR3 sequences represented by the amino acid sequences of SEQ ID NOs: 90, 91, and 92, respectively;
(9) a heavy chain variable domain comprising CDR1, CDR2, and CDR3 sequences represented by the amino acid sequences of SEQ ID NOs: 347, 88, and 358, respectively; and a light chain variable domain comprising CDR1, CDR2, and CDR3 sequences represented by the amino acid sequences of SEQ ID NOs: 90, 91, and 92, respectively;
(10) a heavy chain variable domain comprising CDR1, CDR2, and CDR3 sequences represented by the amino acid sequences of SEQ ID NOs: 347, 88, and 361, respectively; and a light chain variable domain comprising CDR1, CDR2, and CDR3 sequences represented by the amino acid sequences of SEQ ID NOs: 90, 91, and 92, respectively;
(11) a heavy chain variable domain comprising CDR1, CDR2, and CDR3 sequences represented by the amino acid sequences of SEQ ID NOs: 347, 88, and 364, respectively; and a light chain variable domain comprising CDR1, CDR2, and CDR3 sequences represented by the amino acid sequences of SEQ ID NOs: 90, 91, and 92, respectively;
(12) a heavy chain variable domain comprising CDR1, CDR2, and CDR3 sequences represented by the amino acid sequences of SEQ ID NOs: 347, 88, and 367, respectively; and a light chain variable domain comprising CDR1, CDR2, and CDR3 sequences represented by the amino acid sequences of SEQ ID NOs: 90, 91, and 92, respectively; or
(13) a heavy chain variable domain comprising CDR1, CDR2, and CDR3 sequences represented by the amino acid sequences of SEQ ID NOs: 87, 88, and 354, respectively; and a light chain variable domain comprising CDR1, CDR2, and CDR3 sequences represented by the amino acid sequences of SEQ ID NOs: 90, 91, and 92, respectively.

16. The protein according any one of claims 1-14, wherein the first antigen-binding site that binds NKG2D comprises:
(1) a heavy chain variable domain comprising an amino acid sequence at least 90% identical to SEQ ID NO:351 and a light chain variable domain comprising an amino acid sequence at least 90% identical to SEQ ID NO:86;
(2) a heavy chain variable domain comprising an amino acid sequence at least 90% identical to SEQ ID NO:85 and a light chain variable domain comprising an amino acid sequence at least 90% identical to SEQ ID NO:86;
(3) a heavy chain variable domain comprising an amino acid sequence at least 90% identical to SEQ ID NO:77 and a light chain variable domain comprising an amino acid sequence at least 90% identical to SEQ ID NO:78;
(4) a heavy chain variable domain comprising an amino acid sequence at least 90% identical to SEQ ID NO:69 and a light chain variable domain comprising an amino acid sequence at least 90% identical to SEQ ID NO:70;
(5) a heavy chain variable domain comprising an amino acid sequence at least 90% identical to SEQ ID NO:61 and a light chain variable domain comprising an amino acid sequence at least 90% identical to SEQ ID NO:62;
(6) a heavy chain variable domain comprising an amino acid sequence at least 90% identical to SEQ ID NO:93 and a light chain variable domain comprising an amino acid sequence at least 90% identical to SEQ ID NO:94;
(7) a heavy chain variable domain comprising an amino acid sequence at least 90% identical to SEQ ID NO:353 and a light chain variable domain comprising an amino acid sequence at least 90% identical to SEQ ID NO:86;
(8) a heavy chain variable domain comprising an amino acid sequence at least 90% identical to SEQ ID NO:356 and a light chain variable domain comprising an amino acid sequence at least 90% identical to SEQ ID NO:86;
(9) a heavy chain variable domain comprising an amino acid sequence at least 90% identical to SEQ ID NO:359 and a light chain variable domain comprising an amino acid sequence at least 90% identical to SEQ ID NO:86;
(10) a heavy chain variable domain comprising an amino acid sequence at least 90% identical to SEQ ID NO:362 and a light chain variable domain comprising an amino acid sequence at least 90% identical to SEQ ID NO:86; or
(11) a heavy chain variable domain comprising an amino acid sequence at least 90% identical to SEQ ID NO:365 and a light chain variable domain comprising an amino acid sequence at least 90% identical to SEQ ID NO:86.

17. The protein according any one of claims 1-16, wherein the second antigen-binding site that binds B7-H3 comprises a heavy chain variable domain comprising heavy chain CDR1 (CDRH1), heavy chain CDR2 (CDRH2), and heavy chain CDR3 (CDRH3), and a light chain variable domain comprising light chain CDR1 (CDRL1), light chain CDR2 (CDRL2), and light chain CDR3 (CDRL3), wherein the amino acid sequences of CDRH1, CDRH2, CDRH3, CDRL1, CDRL2, and CDRL3 are set forth in SEQ ID NOs: 110, 111, 112, 114, 115, and 116; 118, 119, 120, 122, 123, and 124; 371, 372, 373, 374, 375, and 376; or 379, 380, 381, 382, 383, and 384, respectively.

18. The protein according any one of claims 1-17, wherein the second antigen-binding site that binds B7-H3 comprises:
(a) a heavy chain variable domain comprising an amino acid sequence at least 90% identical to SEQ ID NO:109 and a light chain variable domain comprising an amino acid sequence at least 90% identical to SEQ ID NO:113;
(b) a heavy chain variable domain comprising an amino acid sequence at least 90% identical to SEQ ID NO:117 and a light chain variable domain comprising an amino acid sequence at least 90% identical to SEQ ID NO:121;
(c) a heavy chain variable domain comprising an amino acid sequence at least 90% identical to SEQ ID NO:369 and a light chain variable domain comprising an amino acid sequence at least 90% identical to SEQ ID NO:370;
(d) a heavy chain variable domain comprising an amino acid sequence at least 90% identical to SEQ ID NO:377 and a light chain variable domain comprising an amino acid sequence at least 90% identical to SEQ ID NO:378;
(e) a heavy chain variable domain comprising an amino acid sequence at least 90% identical to SEQ ID NO:386 and a light chain variable domain comprising an amino acid sequence at least 90% identical to SEQ ID NO:387;
(f) a heavy chain variable domain comprising an amino acid sequence at least 90% identical to SEQ ID NO:388 and a light chain variable domain comprising an amino acid sequence at least 90% identical to SEQ ID NO:389; or
(g) a heavy chain variable domain comprising an amino acid sequence at least 90% identical to SEQ ID NO:390 and a light chain variable domain comprising an amino acid sequence at least 90% identical to SEQ ID NO:391.

19. A protein according to any one of claims 1-13 or 15-18 comprising a sequence selected from SEQ ID NOs: 329, 330, 333, 334, 335, and 336.

20. A protein according to any one of claims 1-13 comprising an scFv linked to an antibody Fc domain, wherein the scFv linked to the antibody Fc domain is represented by a sequence selected from SEQ ID NO:330, SEQ ID NO:334, and SEQ ID NO:336.

21. A protein according to any one of claims 1-13 comprising a sequence of SEQ ID NO:329, SEQ ID NO:333, or SEQ ID NO:335.

22. A protein according comprising a sequence at least 90% identical to an amino acid sequence of SEQ ID NO:329, SEQ ID NO:333, or SEQ ID NO:335.

23. A protein according comprising a sequence at least 95% identical to an amino acid sequence of SEQ ID NO:329, SEQ ID NO:333, or SEQ ID NO:335.

24. A protein according comprising a sequence at least 99% identical to an amino acid sequence of SEQ ID NO:329, SEQ ID NO:333, or SEQ ID NO:335.

25. A protein comprising a sequence at least 90% identical to an amino acid sequence selected from SEQ ID NO:330, SEQ ID NO:334, or SEQ ID NO:336.

26. A protein comprising a sequence at least 95% identical to an amino acid sequence selected from SEQ ID NO:330, SEQ ID NO:334, or SEQ ID NO:336.

27. A protein comprising a sequence at least 99% identical to an amino acid sequence selected from SEQ ID NO:330, SEQ ID NO:334, or SEQ ID NO:336.

28. A protein comprising:
(a) a first antigen-binding site that binds NKG2D;
(b) a second antigen-binding site that binds a tumor-associated antigen L1CAM; and
(c) an antibody Fc domain or a portion thereof sufficient to bind CD16, or a third antigen-binding site that binds CD16.

29. A protein comprising:
(a) a first antigen-binding site that binds NKG2D;
(b) a second antigen-binding site that binds a tumor-associated antigen selected from the group consisting of FLT1, KDR, TNC, TNN, CSPG4, BST1, SELP, CD200, INSR (HHF5), ITGA6, MELTF, PECAM1, and SLC1A5; and
(c) an antibody Fc domain or a portion thereof sufficient to bind CD16, or a third antigen-binding site that binds CD16.

30. A protein of claim 14, 28, or 29 further comprising an additional antigen-binding site that binds the same tumor-associated antigen as the second antigen-binding site.

31. A protein of claim 14, 28, 29, or 30, wherein the first antigen-binding site that binds NKG2D is a single-chain variable fragment (scFv), and the second and/or the additional antigen-binding site that binds a tumor-associated antigen is an Fab fragment.

32. A protein of claim 14, 28, 29, or 30, wherein the first antigen-binding site that binds NKG2D is an scFv, and the second and/or the additional antigen-binding site that binds a tumor-associated antigen is an scFv.

33. A protein of claim 14, 28, or 29, wherein the first antigen-binding site that binds NKG2D is an Fab fragment, and the second antigen-binding site that binds a tumor-associated antigen is an scFv.

34. A protein of claim 14, 28, or 29, wherein the first antigen-binding site that binds NKG2D is an scFv, and the second antigen-binding site that binds a tumor-associated antigen is an Fab fragment.

35. The protein of any one of claims 14 and 28-34, wherein the first antigen-binding site binds to NKG2D in humans.

36. The protein of any one of claims 14 and 28-35, wherein the first, the second, and/or the additional antigen-binding site comprises a heavy chain variable domain and a light chain variable domain.

37. The protein of any one of claims 31-34, wherein the scFv the scFv that binds the tumor-associated antigen and/or the scFv that binds NKG2D is linked to an antibody constant domain or a portion thereof sufficient to bind CD16, via a hinge comprising Ala-Ser or Gly-Ala-Ser, wherein the scFv comprises a heavy chain variable domain and a light chain variable domain.

38. The protein according to claim 36 or 37, wherein the heavy chain variable domain forms a disulfide bridge with the light chain variable domain.

39. The protein according to claim 38, wherein the disulfide bridge is formed between C44 from the heavy chain variable domain and C100 from the light chain variable domain.

40. The protein according to any one of claims 37-39, wherein within the scFv the heavy chain variable domain is linked to the light chain variable domain via a flexible linker.

41. The protein according to claim 40, wherein within in the scFv the flexible linker comprises (GlyGlyGlyGlySer)₄ ((G4S)₄) (SEQ ID NO:126).

42. The protein according to any one of claims 37-41, wherein within the scFv the heavy chain variable domain is positioned at the N-terminus or the C-terminus of the light chain variable domain.

43. The protein according to any one of claims 37-42, wherein within the scFv the hinge further comprises amino acid sequence Thr-Lys-Gly.

44. The protein according any one of claims 14 and 28-43, wherein the first antigen-binding site that binds NKG2D comprises a heavy chain variable domain at least 90% identical to an amino acid sequence selected from: SEQ ID NO:1, SEQ ID NO:41, SEQ ID NO:49, SEQ ID NO:57, SEQ ID NO:59, SEQ ID NO:61, SEQ ID NO:69, SEQ ID NO:77, SEQ ID NO:85, SEQ ID NO:351, SEQ ID NO:353, SEQ ID NO:356, SEQ ID NO:359, SEQ ID NO:362, SEQ ID NO:365, and SEQ ID NO:93.

45. The protein according any one of claims 14 and 28-43, wherein the first antigen-binding site that binds NKG2D comprises a heavy chain variable domain at least 90% identical to SEQ ID NO:351 and a light chain variable domain at least 90% identical to SEQ ID NO:86.

46. The protein according any one of claims 14 and 28-43, wherein the first antigen-binding site that binds NKG2D comprises a heavy chain variable domain at least 90% identical to SEQ ID NO:365 and a light chain variable domain at least 90% identical to SEQ ID NO:86.

47. The protein according any one of claims 14 and 28-43, wherein the first antigen-binding site that binds NKG2D comprises a heavy chain variable domain at least 90% identical to SEQ ID NO:41 and a light chain variable domain at least 90% identical to SEQ ID NO:42.

48. The protein according any one of claims 14 and 28-43, wherein the first antigen-binding site that binds NKG2D comprises a heavy chain variable domain at least 90% identical to SEQ ID NO:49 and a light chain variable domain at least 90% identical to SEQ ID NO:50.

49. The protein according any one of claims 14 and 28-43, wherein the first antigen-binding site that binds NKG2D comprises a heavy chain variable domain at least 90% identical to SEQ ID NO:57 and a light chain variable domain at least 90% identical to SEQ ID NO:58.

50. The protein according any one of claims 14 and 28-43, wherein the first antigen-binding site that binds NKG2D comprises a heavy chain variable domain at least 90% identical to SEQ ID NO:59 and a light chain variable domain at least 90% identical to SEQ ID NO:60.

51. The protein according any one of claims 14 and 28-43, wherein the first antigen-binding site that binds NKG2D comprises a heavy chain variable domain at least 90% identical to SEQ ID NO:61 and a light chain variable domain at least 90% identical to SEQ ID NO:62.

52. The protein according any one of claims 14 and 28-43, wherein the first antigen-binding site that binds NKG2D comprises a heavy chain variable domain at least 90% identical to SEQ ID NO:69 and a light chain variable domain at least 90% identical to SEQ ID NO:70.

53. The protein according any one of claims 14 and 28-43, wherein the first antigen-binding site that binds NKG2D comprises a heavy chain variable domain at least 90% identical to SEQ ID NO:77 and a light chain variable domain at least 90% identical to SEQ ID NO:78.

54. The protein according any one of claims 14 and 28-43, wherein the first antigen-binding site that binds NKG2D comprises a heavy chain variable domain at least 90% identical to SEQ ID NO:85 and a light chain variable domain at least 90% identical to SEQ ID NO:86.

55. The protein according any one of claims 14 and 28-43, wherein the first antigen-binding site that binds NKG2D comprises a heavy chain variable domain at least 90% identical to SEQ ID NO:93 and a light chain variable domain at least 90% identical to SEQ ID NO:94.

56. The protein according any one of claims 14 and 28-43, wherein the first antigen-binding site that binds NKG2D comprises a heavy chain variable domain at least 90% identical to SEQ ID NO:101 and alight chain variable domain at least 90% identical to SEQ ID NO:102.

57. The protein according any one of claims 14 and 28-43, wherein the first antigen-binding site that binds NKG2D comprises a heavy chain variable domain at least 90% identical to SEQ ID NO:103 and a light chain variable domain at least 90% identical to SEQ ID NO:104.

58. The protein according to any one of claims 14 and 30-57, wherein the second antigen-binding site binds B7-H3, the heavy chain variable domain of the second antigen-binding site comprises an amino acid sequence at least 90% identical to SEQ ID NO:109 or 386 and the light chain variable domain of the second antigen-binding site comprises an amino acid sequence at least 90% identical to SEQ ID NO:113 or 387.

59. The protein according to any one of claims 14 and 30-57, wherein the second antigen-binding site binds B7-H3, the heavy chain variable domain of the second antigen-binding site comprises an amino acid sequence at least 90% identical to SEQ ID NO:117 and the light chain variable domain of the second antigen-binding site comprises an amino acid sequence at least 90% identical to SEQ ID NO:121.

60. The protein according to any one of claims 14 and 30-57, wherein the second antigen-binding site binds B7-H3, the heavy chain variable domain of the second antigen-binding site comprises an amino acid sequence at least 90% identical to SEQ ID NO:369 or 388 and the light chain variable domain of the second antigen-binding site comprises an amino acid sequence at least 90% identical to SEQ ID NO:370 or 389.

61. The protein according to any one of claims 14 and 30-57, wherein the second antigen-binding site binds B7-H3, the heavy chain variable domain of the second antigen-binding site comprises an amino acid sequence at least 90% identical to SEQ ID NO:377 or 390 and the light chain variable domain of the second antigen-binding site comprises an amino acid sequence at least 90% identical to SEQ ID NO:378 or 391.

62. The protein according to any one of claims 28 and 30-57, wherein the second antigen-binding site binds L1CAM, the heavy chain variable domain of the second antigen-binding site comprises an amino acid sequence at least 90% identical to SEQ ID NO:133 and the light chain variable domain of the second antigen-binding site comprises an amino acid sequence at least 90% identical to SEQ ID NO:137.

63. The protein according to any one of claims 28 and 30-57, wherein the second antigen-binding site binds L1CAM, the heavy chain variable domain of the second antigen-binding site comprises an amino acid sequence at least 90% identical to SEQ ID NO:141 and the light chain variable domain of the second antigen-binding site comprises an amino acid sequence at least 90% identical to SEQ ID NO:145.

64. The protein according to any one of claims 29-57, wherein the second antigen-binding site binds FLT1, the heavy chain variable domain of the second antigen-binding site comprises an amino acid sequence at least 90% identical to SEQ ID NO:150 and the light chain variable domain of the second antigen-binding site comprises an amino acid sequence at least 90% identical to SEQ ID NO:154.

65. The protein according to any one of claims 29-57, wherein the second antigen-binding site binds FLT1, the heavy chain variable domain of the second antigen-binding site comprises an amino acid sequence at least 90% identical to SEQ ID NO:158 and the light chain variable domain of the second antigen-binding site comprises an amino acid sequence at least 90% identical to SEQ ID NO:162.

66. The protein according to any one of claims 29-57, wherein the second antigen-binding site binds KDR, the heavy chain variable domain of the second antigen-binding site comprises an amino acid sequence at least 90% identical to SEQ ID NO:166 and the light chain variable domain of the second antigen-binding site comprises an amino acid sequence at least 90% identical to SEQ ID NO:170.

67. The protein according to any one of claims 29-57, wherein the second antigen-binding site binds KDR, the heavy chain variable domain of the second antigen-binding site comprises an amino acid sequence at least 90% identical to SEQ ID NO:174 and the light chain variable domain of the second antigen-binding site comprises an amino acid sequence at least 90% identical to SEQ ID NO:178.

68. The protein according to any one of claims 29-57, wherein the second antigen-binding site binds TNC, the heavy chain variable domain of the second antigen-binding site comprises an amino acid sequence at least 90% identical to SEQ ID NO:182 and the light chain variable domain of the second antigen-binding site comprises an amino acid sequence at least 90% identical to SEQ ID NO:186.

69. The protein according to any one of claims 29-57, wherein the second antigen-binding site binds TNC, the heavy chain variable domain of the second antigen-binding site comprises an amino acid sequence at least 90% identical to SEQ ID NO:190 and the light chain variable domain of the second antigen-binding site comprises an amino acid sequence at least 90% identical to SEQ ID NO:194.

70. The protein according to any one of claims 29-57, wherein the second antigen-binding site binds CSPG4, the heavy chain variable domain of the second antigen-binding site comprises an amino acid sequence at least 90% identical to SEQ ID NO:198 and the light chain variable domain of the second antigen-binding site comprises an amino acid sequence at least 90% identical to SEQ ID NO:202.

71. The protein according to any one of claims 29-57, wherein the second antigen-binding site binds CSPG4, the heavy chain variable domain of the second antigen-binding site comprises an amino acid sequence at least 90% identical to SEQ ID NO:206 and the light chain variable domain of the second antigen-binding site comprises an amino acid sequence at least 90% identical to SEQ ID NO:210.

72. The protein according to any one of claims 29-57, wherein the second antigen-binding site binds BST1, the heavy chain variable domain of the second antigen-binding site comprises an amino acid sequence at least 90% identical to SEQ ID NO:214 and the light chain variable domain of the second antigen-binding site comprises an amino acid sequence at least 90% identical to SEQ ID NO:218.

73. The protein according to any one of claims 29-57, wherein the second antigen-binding site binds BST1, the heavy chain variable domain of the second antigen-binding site comprises an amino acid sequence at least 90% identical to SEQ ID NO:222 and the light chain variable domain of the second antigen-binding site comprises an amino acid sequence at least 90% identical to SEQ ID NO:226.

74. The protein according to any one of claims 29-57, wherein the second antigen-binding site binds SELP, the heavy chain variable domain of the second antigen-binding site comprises an amino acid sequence at least 90% identical to SEQ ID NO:230 and the light chain variable domain of the second antigen-binding site comprises an amino acid sequence at least 90% identical to SEQ ID NO:234.

75. The protein according to any one of claims 29-57, wherein the second antigen-binding site binds SELP, the heavy chain variable domain of the second antigen-binding site comprises an amino acid sequence at least 90% identical to SEQ ID NO:238 and the light chain variable domain of the second antigen-binding site comprises an amino acid sequence at least 90% identical to SEQ ID NO:242.

76. The protein according to any one of claims 29-57, wherein the second antigen-binding site binds CD200, the heavy chain variable domain of the second antigen-binding site comprises an amino acid sequence at least 90% identical to SEQ ID NO:246 and the light chain variable domain of the second antigen-binding site comprises an amino acid sequence at least 90% identical to SEQ ID NO:250.

77. The protein according to any one of claims 29-57, wherein the second antigen-binding site binds INSR, the heavy chain variable domain of the second antigen-binding site comprises an amino acid sequence at least 90% identical to SEQ ID NO:254 and the light chain variable domain of the second antigen-binding site comprises an amino acid sequence at least 90% identical to SEQ ID NO:258.

78. The protein according to any one of claims 29-57, wherein the second antigen-binding site binds INSR, the heavy chain variable domain of the second antigen-binding site comprises an amino acid sequence at least 90% identical to SEQ ID NO:262 and the light chain variable domain of the second antigen-binding site comprises an amino acid sequence at least 90% identical to SEQ ID NO:266.

79. The protein according to any one of claims 29-57, wherein the second antigen-binding site binds ITGA6, the heavy chain variable domain of the second antigen-binding site comprises an amino acid sequence at least 90% identical to SEQ ID NO:270 and the light chain variable domain of the second antigen-binding site comprises an amino acid sequence at least 90% identical to SEQ ID NO:274.

80. The protein according to any one of claims 29-57, wherein the second antigen-binding site binds MELTF, the heavy chain variable domain of the second antigen-binding site comprises an amino acid sequence at least 90% identical to SEQ ID NO:284 and the light chain variable domain of the second antigen-binding site comprises an amino acid sequence at least 90% identical to SEQ ID NO:288.

81. The protein according to any one of claims 29-57, wherein the second antigen-binding site binds MELTF, the heavy chain variable domain of the second antigen-binding site comprises an amino acid sequence at least 90% identical to SEQ ID NO:292 and the light chain variable domain of the second antigen-binding site comprises an amino acid sequence at least 90% identical to SEQ ID NO:296.

82. The protein according to any one of claims 29-57, wherein the second antigen-binding site binds SLC1A5, the heavy chain variable domain of the second antigen-binding site comprises an amino acid sequence at least 90% identical to SEQ ID NO:300 and the light chain variable domain of the second antigen-binding site comprises an amino acid sequence at least 90% identical to SEQ ID NO:304.

83. The protein according to any one of claims 29-57, wherein the second antigen-binding site binds SLC1A5, the heavy chain variable domain of the second antigen-binding site comprises an amino acid sequence at least 90% identical to SEQ ID NO:308 and the light chain variable domain of the second antigen-binding site comprises an amino acid sequence at least 90% identical to SEQ ID NO:312.

84. The protein according to any one of claims 29-57 and 62, wherein the second antigen-binding site binds L1CAM, and wherein the heavy chain variable domain of the second antigen-binding site comprises an amino acid sequence including:
(a) a heavy chain CDR1 sequence identical to the amino acid sequence of SEQ ID NO:134;
(b) a heavy chain CDR2 sequence identical to the amino acid sequence of SEQ ID NO:135; and
(c) a heavy chain CDR3 sequence identical to the amino acid sequence of SEQ ID NO:136,
and wherein the light chain variable domain of the second antigen-binding site comprises an amino acid sequence including:
(d) a light chain CDR1 sequence identical to the amino acid sequence of SEQ ID NO:138;
(e) a light chain CDR2 sequence identical to the amino acid sequence of SEQ ID NO:139; and
(f) a light chain CDR3 sequence identical to the amino acid sequence of SEQ ID NO:140.

85. The protein according to any one of claims 29-57 and 63, wherein the second antigen-binding site binds L1CAM, and wherein the heavy chain variable domain of the second antigen-binding site comprises an amino acid sequence including:
(a) a heavy chain CDR1 sequence identical to the amino acid sequence of SEQ ID NO:142;
(b) a heavy chain CDR2 sequence identical to the amino acid sequence of SEQ ID NO:143; and
(c) a heavy chain CDR3 sequence identical to the amino acid sequence of SEQ ID NO:144,
and wherein the light chain variable domain of the second antigen-binding site comprises an amino acid sequence including:
(d) a light chain CDR1 sequence identical to the amino acid sequence of SEQ ID NO:146;
(e) a light chain CDR2 sequence identical to the amino acid sequence of SEQ ID NO:147; and
(f) a light chain CDR3 sequence identical to the amino acid sequence of SEQ ID NO:148.

86. The protein according to any one of claims 29-57 and 64, wherein the second antigen-binding site binds FLT1, and wherein the heavy chain variable domain of the second antigen-binding site comprises an amino acid sequence including:
(a) a heavy chain CDR1 sequence identical to the amino acid sequence of SEQ ID NO:151;
(b) a heavy chain CDR2 sequence identical to the amino acid sequence of SEQ ID NO:152; and
(c) a heavy chain CDR3 sequence identical to the amino acid sequence of SEQ ID NO:153,
and wherein the light chain variable domain of the second antigen-binding site comprises an amino acid sequence including:
(d) a light chain CDR1 sequence identical to the amino acid sequence of SEQ ID NO:155;
(e) a light chain CDR2 sequence identical to the amino acid sequence of SEQ ID NO:156; and
(f) a light chain CDR3 sequence identical to the amino acid sequence of SEQ ID NO:157.

87. The protein according to any one of claims 29-57 and 65, wherein the second antigen-binding site binds FLT1, and wherein the heavy chain variable domain of the second antigen-binding site comprises an amino acid sequence including:
(a) a heavy chain CDR1 sequence identical to the amino acid sequence of SEQ ID NO:159;
(b) a heavy chain CDR2 sequence identical to the amino acid sequence of SEQ ID NO:160; and
(c) a heavy chain CDR3 sequence identical to the amino acid sequence of SEQ ID NO:161,
and wherein the light chain variable domain of the second antigen-binding site comprises an amino acid sequence including:
(d) a light chain CDR1 sequence identical to the amino acid sequence of SEQ ID NO:163;
(e) a light chain CDR2 sequence identical to the amino acid sequence of SEQ ID NO:164; and
(f) a light chain CDR3 sequence identical to the amino acid sequence of SEQ ID NO:165.

88. The protein according to any one of claims 29-57 and 66, wherein the second antigen-binding site binds KDR, and wherein the heavy chain variable domain of the second antigen-binding site comprises an amino acid sequence including:
(a) a heavy chain CDR1 sequence identical to the amino acid sequence of SEQ ID NO:167;
(b) a heavy chain CDR2 sequence identical to the amino acid sequence of SEQ ID NO:168; and
(c) a heavy chain CDR3 sequence identical to the amino acid sequence of SEQ ID NO:169,
and wherein the light chain variable domain of the second antigen-binding site comprises an amino acid sequence including:
(d) a light chain CDR1 sequence identical to the amino acid sequence of SEQ ID NO:171;
(e) a light chain CDR2 sequence identical to the amino acid sequence of SEQ ID NO:172; and
(f) a light chain CDR3 sequence identical to the amino acid sequence of SEQ ID NO:173.

89. The protein according to any one of claims 29-57 and 67, wherein the second antigen-binding site binds KDR, and wherein the heavy chain variable domain of the second antigen-binding site comprises an amino acid sequence including:
(a) a heavy chain CDR1 sequence identical to the amino acid sequence of SEQ ID NO:175;
(b) a heavy chain CDR2 sequence identical to the amino acid sequence of SEQ ID NO:176; and
(c) a heavy chain CDR3 sequence identical to the amino acid sequence of SEQ ID NO:177,
and wherein the light chain variable domain of the second antigen-binding site comprises an amino acid sequence including:
(d) a light chain CDR1 sequence identical to the amino acid sequence of SEQ ID NO:179;
(e) a light chain CDR2 sequence identical to the amino acid sequence of SEQ ID NO:180; and
(f) a light chain CDR3 sequence identical to the amino acid sequence of SEQ ID NO:181.

90. The protein according to any one of claims 29-57 and 68, wherein the second antigen-binding site binds TNC, and wherein the heavy chain variable domain of the second antigen-binding site comprises an amino acid sequence including:
(a) a heavy chain CDR1 sequence identical to the amino acid sequence of SEQ ID NO:183;
(b) a heavy chain CDR2 sequence identical to the amino acid sequence of SEQ ID NO:184; and
(c) a heavy chain CDR3 sequence identical to the amino acid sequence of SEQ ID NO:185,
and wherein the light chain variable domain of the second antigen-binding site comprises an amino acid sequence including:
(d) a light chain CDR1 sequence identical to the amino acid sequence of SEQ ID NO:187;
(e) a light chain CDR2 sequence identical to the amino acid sequence of SEQ ID NO:188; and
(f) a light chain CDR3 sequence identical to the amino acid sequence of SEQ ID NO:189.

91. The protein according to any one of claims 29-57 and 69, wherein the second antigen-binding site binds TNC, and wherein the heavy chain variable domain of the second antigen-binding site comprises an amino acid sequence including:
(a) a heavy chain CDR1 sequence identical to the amino acid sequence of SEQ ID NO:191;
(b) a heavy chain CDR2 sequence identical to the amino acid sequence of SEQ ID NO:192; and
(c) a heavy chain CDR3 sequence identical to the amino acid sequence of SEQ ID NO:193,
and wherein the light chain variable domain of the second antigen-binding site comprises an amino acid sequence including:
(d) a light chain CDR1 sequence identical to the amino acid sequence of SEQ ID NO:195;
(e) a light chain CDR2 sequence identical to the amino acid sequence of SEQ ID NO:196; and
(f) a light chain CDR3 sequence identical to the amino acid sequence of SEQ ID NO:197.

92. The protein according to any one of claims 29-57 and 70, wherein the second antigen-binding site binds CSPG4, and wherein the heavy chain variable domain of the second antigen-binding site comprises an amino acid sequence including:
(a) a heavy chain CDR1 sequence identical to the amino acid sequence of SEQ ID NO:199;
(b) a heavy chain CDR2 sequence identical to the amino acid sequence of SEQ ID NO:200; and
(c) a heavy chain CDR3 sequence identical to the amino acid sequence of SEQ ID NO:201,
and wherein the light chain variable domain of the second antigen-binding site comprises an amino acid sequence including:
(d) a light chain CDR1 sequence identical to the amino acid sequence of SEQ ID NO:203;
(e) a light chain CDR2 sequence identical to the amino acid sequence of SEQ ID NO:204; and
(f) a light chain CDR3 sequence identical to the amino acid sequence of SEQ ID NO:205.

93. The protein according to any one of claims 29-57 and 71, wherein the second antigen-binding site binds CSPG4, and wherein the heavy chain variable domain of the second antigen-binding site comprises an amino acid sequence including:
(a) a heavy chain CDR1 sequence identical to the amino acid sequence of SEQ ID NO:207;
(b) a heavy chain CDR2 sequence identical to the amino acid sequence of SEQ ID NO:208; and
(c) a heavy chain CDR3 sequence identical to the amino acid sequence of SEQ ID NO:209,
and wherein the light chain variable domain of the second antigen-binding site comprises an amino acid sequence including:
(d) a light chain CDR1 sequence identical to the amino acid sequence of SEQ ID NO:211;
(e) a light chain CDR2 sequence identical to the amino acid sequence of SEQ ID NO:212; and
(f) a light chain CDR3 sequence identical to the amino acid sequence of SEQ ID NO:213.

94. The protein according to any one of claims 29-57 and 72, wherein the second antigen-binding site binds BST1, and wherein the heavy chain variable domain of the second antigen-binding site comprises an amino acid sequence including:
(a) a heavy chain CDR1 sequence identical to the amino acid sequence of SEQ ID NO:215;
(b) a heavy chain CDR2 sequence identical to the amino acid sequence of SEQ ID NO:216; and
(c) a heavy chain CDR3 sequence identical to the amino acid sequence of SEQ ID NO:217,
and wherein the light chain variable domain of the second antigen-binding site comprises an amino acid sequence including:
(d) a light chain CDR1 sequence identical to the amino acid sequence of SEQ ID NO:219;
(e) a light chain CDR2 sequence identical to the amino acid sequence of SEQ ID NO:220; and
(f) a light chain CDR3 sequence identical to the amino acid sequence of SEQ ID NO:221.

95. The protein according to any one of claims 29-57 and 73, wherein the second antigen-binding site binds BST1, and wherein the heavy chain variable domain of the second antigen-binding site comprises an amino acid sequence including:
(a) a heavy chain CDR1 sequence identical to the amino acid sequence of SEQ ID NO:223;
(b) a heavy chain CDR2 sequence identical to the amino acid sequence of SEQ ID NO:224; and
(c) a heavy chain CDR3 sequence identical to the amino acid sequence of SEQ ID NO:225,
and wherein the light chain variable domain of the second antigen-binding site comprises an amino acid sequence including:
(d) a light chain CDR1 sequence identical to the amino acid sequence of SEQ ID NO:227;
(e) a light chain CDR2 sequence identical to the amino acid sequence of SEQ ID NO:228; and
(f) a light chain CDR3 sequence identical to the amino acid sequence of SEQ ID NO:229.

96. The protein according to any one of claims 29-57 and 74, wherein the second antigen-binding site binds SELP, and wherein the heavy chain variable domain of the second antigen-binding site comprises an amino acid sequence including:
(a) a heavy chain CDR1 sequence identical to the amino acid sequence of SEQ ID NO:231;
(b) a heavy chain CDR2 sequence identical to the amino acid sequence of SEQ ID NO:232; and
(c) a heavy chain CDR3 sequence identical to the amino acid sequence of SEQ ID NO:233,
and wherein the light chain variable domain of the second antigen-binding site comprises an amino acid sequence including:
(d) a light chain CDR1 sequence identical to the amino acid sequence of SEQ ID NO:235;
(e) a light chain CDR2 sequence identical to the amino acid sequence of SEQ ID NO:236; and
(f) a light chain CDR3 sequence identical to the amino acid sequence of SEQ ID NO:237.

97. The protein according to any one of claims 29-57 and 75, wherein the second antigen-binding site binds SELP, and wherein the heavy chain variable domain of the second antigen-binding site comprises an amino acid sequence including:
(a) a heavy chain CDR1 sequence identical to the amino acid sequence of SEQ ID NO:239;
(b) a heavy chain CDR2 sequence identical to the amino acid sequence of SEQ ID NO:240; and
(c) a heavy chain CDR3 sequence identical to the amino acid sequence of SEQ ID NO:241,
and wherein the light chain variable domain of the second antigen-binding site comprises an amino acid sequence including:
(d) a light chain CDR1 sequence identical to the amino acid sequence of SEQ ID NO:243;
(e) a light chain CDR2 sequence identical to the amino acid sequence of SEQ ID NO:244; and
(f) a light chain CDR3 sequence identical to the amino acid sequence of SEQ ID NO:245.

98. The protein according to any one of claims 29-57 and 76, wherein the second antigen-binding site binds CD200, and wherein the heavy chain variable domain of the second antigen-binding site comprises an amino acid sequence including:
(a) a heavy chain CDR1 sequence identical to the amino acid sequence of SEQ ID NO:247;
(b) a heavy chain CDR2 sequence identical to the amino acid sequence of SEQ ID NO:248; and
(c) a heavy chain CDR3 sequence identical to the amino acid sequence of SEQ ID NO:249,
and wherein the light chain variable domain of the second antigen-binding site comprises an amino acid sequence including:
(d) a light chain CDR1 sequence identical to the amino acid sequence of SEQ ID NO:251;
(e) a light chain CDR2 sequence identical to the amino acid sequence of SEQ ID NO:252; and
(f) a light chain CDR3 sequence identical to the amino acid sequence of SEQ ID NO:253.

99. The protein according to any one of claims 29-57 and 77, wherein the second antigen-binding site binds INSR, and wherein the heavy chain variable domain of the second antigen-binding site comprises an amino acid sequence including:
(a) a heavy chain CDR1 sequence identical to the amino acid sequence of SEQ ID NO:255;
(b) a heavy chain CDR2 sequence identical to the amino acid sequence of SEQ ID NO:256; and
(c) a heavy chain CDR3 sequence identical to the amino acid sequence of SEQ ID NO:257,
and wherein the light chain variable domain of the second antigen-binding site comprises an amino acid sequence including:
(d) a light chain CDR1 sequence identical to the amino acid sequence of SEQ ID NO:259;
(e) a light chain CDR2 sequence identical to the amino acid sequence of SEQ ID NO:260; and
(f) a light chain CDR3 sequence identical to the amino acid sequence of SEQ ID NO:261.

100. The protein according to any one of claims 29-57 and 78, wherein the second antigen-binding site binds INSR, and wherein the heavy chain variable domain of the second antigen-binding site comprises an amino acid sequence including:
(a) a heavy chain CDR1 sequence identical to the amino acid sequence of SEQ ID NO:263;
(b) a heavy chain CDR2 sequence identical to the amino acid sequence of SEQ ID NO:264; and
(c) a heavy chain CDR3 sequence identical to the amino acid sequence of SEQ ID NO:265,
and wherein the light chain variable domain of the second antigen-binding site comprises an amino acid sequence including:
(d) a light chain CDR1 sequence identical to the amino acid sequence of SEQ ID NO:267;
(e) a light chain CDR2 sequence identical to the amino acid sequence of SEQ ID NO:268; and
(f) a light chain CDR3 sequence identical to the amino acid sequence of SEQ ID NO:269.

101. The protein according to any one of claims 29-57 and 79, wherein the second antigen-binding site binds ITGA6, and wherein the heavy chain variable domain of the second antigen-binding site comprises an amino acid sequence including:
(a) a heavy chain CDR1 sequence identical to the amino acid sequence of SEQ ID NO:271;
(b) a heavy chain CDR2 sequence identical to the amino acid sequence of SEQ ID NO:272; and
(c) a heavy chain CDR3 sequence identical to the amino acid sequence of SEQ ID NO:273,
and wherein the light chain variable domain of the second antigen-binding site comprises an amino acid sequence including:
(d) a light chain CDR1 sequence identical to the amino acid sequence of SEQ ID NO:275;
(e) a light chain CDR2 sequence identical to the amino acid sequence of SEQ ID NO:276; and
(f) a light chain CDR3 sequence identical to the amino acid sequence of SEQ ID NO:277.

102. The protein according to any one of claims 29-57, wherein the second antigen-binding site binds ITGA6, and wherein the heavy chain variable domain of the second antigen-binding site comprises an amino acid sequence including:
(a) a heavy chain CDR1 sequence identical to the amino acid sequence of SEQ ID NO:278;
(b) a heavy chain CDR2 sequence identical to the amino acid sequence of SEQ ID NO:279; and
(c) a heavy chain CDR3 sequence identical to the amino acid sequence of SEQ ID NO:280,
and wherein the light chain variable domain of the second antigen-binding site comprises an amino acid sequence including:
(d) a light chain CDR1 sequence identical to the amino acid sequence of SEQ ID NO:281;
(e) a light chain CDR2 sequence identical to the amino acid sequence of SEQ ID NO:282; and
(f) a light chain CDR3 sequence identical to the amino acid sequence of SEQ ID NO:283.

103. The protein according to any one of claims 29-57 and 80, wherein the second antigen-binding site binds MELTF, and wherein the heavy chain variable domain of the second antigen-binding site comprises an amino acid sequence including:
(a) a heavy chain CDR1 sequence identical to the amino acid sequence of SEQ ID NO:285;
(b) a heavy chain CDR2 sequence identical to the amino acid sequence of SEQ ID NO:286; and
(c) a heavy chain CDR3 sequence identical to the amino acid sequence of SEQ ID NO:287,
and wherein the light chain variable domain of the second antigen-binding site comprises an amino acid sequence including:
(d) a light chain CDR1 sequence identical to the amino acid sequence of SEQ ID NO:289;
(e) a light chain CDR2 sequence identical to the amino acid sequence of SEQ ID NO:290; and
(f) a light chain CDR3 sequence identical to the amino acid sequence of SEQ ID NO:291.

104. The protein according to any one of claims 29-57 and 81, wherein the second antigen-binding site binds MELTF, and wherein the heavy chain variable domain of the second antigen-binding site comprises an amino acid sequence including:
(a) a heavy chain CDR1 sequence identical to the amino acid sequence of SEQ ID NO:293;
(b) a heavy chain CDR2 sequence identical to the amino acid sequence of SEQ ID NO:294; and
(c) a heavy chain CDR3 sequence identical to the amino acid sequence of SEQ ID NO:295,
and wherein the light chain variable domain of the second antigen-binding site comprises an amino acid sequence including:
(d) a light chain CDR1 sequence identical to the amino acid sequence of SEQ ID NO:297;
(e) a light chain CDR2 sequence identical to the amino acid sequence of SEQ ID NO:298; and
(f) a light chain CDR3 sequence identical to the amino acid sequence of SEQ ID NO:299.

105. The protein according to any one of claims 29-57 and 82, wherein the second antigen-binding site binds SLC1A5, and wherein the heavy chain variable domain of the second antigen-binding site comprises an amino acid sequence including:
(a) a heavy chain CDR1 sequence identical to the amino acid sequence of SEQ ID NO:301;
(b) a heavy chain CDR2 sequence identical to the amino acid sequence of SEQ ID NO:302; and
(c) a heavy chain CDR3 sequence identical to the amino acid sequence of SEQ ID NO:303,
and wherein the light chain variable domain of the second antigen-binding site comprises an amino acid sequence including:
(d) a light chain CDR1 sequence identical to the amino acid sequence of SEQ ID NO:305;
(e) a light chain CDR2 sequence identical to the amino acid sequence of SEQ ID NO:306; and
(f) a light chain CDR3 sequence identical to the amino acid sequence of SEQ ID NO:307.

106. The protein according to any one of claims 29-57 and 83, wherein the second antigen-binding site binds SLC1A5, and wherein the heavy chain variable domain of the second antigen-binding site comprises an amino acid sequence including:
(a) a heavy chain CDR1 sequence identical to the amino acid sequence of SEQ ID NO:309;
(b) a heavy chain CDR2 sequence identical to the amino acid sequence of SEQ ID NO:310; and
(c) a heavy chain CDR3 sequence identical to the amino acid sequence of SEQ ID NO:311,
and wherein the light chain variable domain of the second antigen-binding site comprises an amino acid sequence including:
(d) a light chain CDR1 sequence identical to the amino acid sequence of SEQ ID NO:313;
(e) a light chain CDR2 sequence identical to the amino acid sequence of SEQ ID NO:314; and
(f) a light chain CDR3 sequence identical to the amino acid sequence of SEQ ID NO:315.

107. The protein according to any one of claims 14 and 28-106, wherein the protein comprises an antibody Fc domain or a portion thereof sufficient to bind CD16, wherein the antibody Fc domain comprises hinge and CH2 domains.

108. The protein according to claim 107, wherein the antibody Fc domain comprises hinge and CH2 domains of a human IgG1 antibody.

109. The protein according to claim 107 or 108, wherein the antibody Fc domain comprises an amino acid sequence at least 90% identical to amino acids 234-332 of a human IgG1 antibody.

110. The protein according to claim 109, wherein the antibody Fc domain comprises amino acid sequence at least 90% identical to the Fc domain of human IgG1 and differs at one or more positions selected from the group consisting of Q347, Y349, L351, Q352, S354, E356, E357, K360, Q362, S364, T366, L368, K370, N390, K392, T394, D399, S400, D401, F405, Y407, K409, T411, and K439.

111. A formulation comprising a protein according to any one of the preceding claims and a pharmaceutically acceptable carrier.

112. A cell comprising one or more nucleic acids expressing a protein according to any one of claims 1-110.

113. A method of enhancing tumor cell death, the method comprising exposing tumor cells and natural killer cells to an effective amount of the protein according to any one of claims 1-61 and 107-110, wherein the tumor cells express B7-H3.

114. A method of enhancing tumor cell death, the method comprising exposing tumor cells and natural killer cells to an effective amount of the protein according to any one of claims 1-110, wherein the tumor cells express a tumor-associated antigen selected from B7-H3, L1CAM, FLT1, KDR, TNC, TNN, CSPG4, BST1, SELP, CD200, INSR (HHF5), ITGA6, MELTF, PECAM1, and SLC1A5.

115. A method of treating cancer, wherein the method comprises administering an effective amount of the protein according to any one of claims 1-110 or the formulation according to claim 111 to a patient.

116. The method of claim 115, wherein the second antigen binding site of the protein binds B7-H3, and wherein the cancer is selected from the group consisting of bladder cancer, breast cancer, cervical cancer, glioblastoma, head and neck cancer, lung cancer, liver cancer, melanoma, ovarian cancer, pancreatic cancer, prostate cancer, sarcoma, renal cancer, colorectal cancer, gastric cancer, neuroblastoma, squamous cell carcinoma, and acute myeloid leukemia (AML).

117. The method of claim 115, wherein the second antigen binding site of the protein binds L1CAM, and wherein the cancer is selected from the group consisting of bladder cancer, renal cancer, breast cancer, cervical cancer, sarcoma, lung cancer, head and neck cancer, glioblastoma, neuroblastoma, melanoma, ovarian cancer, endometrial cancer, esophageal cancer, gastric cancer, gastrointestinal stromal tumor (GIST), cholangiocarcinoma, colorectal cancer, pancreatic cancer, and prostate cancer.

118. The method of claim 115, wherein the second antigen binding site of the protein binds FLT1, and wherein the cancer to be treated is selected from the group consisting of renal cancer, gastric cancer, glioma, colorectal cancer, biliary tract cancer, prostate cancer, sarcoma, and breast cancer.

119. The method of claim 115, wherein the second antigen binding site of the protein binds KDR, and wherein the cancer to be treated is selected from the group consisting of renal cancer, gastric cancer, glioma, colorectal cancer, biliary tract cancer, lung cancer, melanoma, liver cancer, sarcoma, breast cancer, mesothelioma, and thyroid cancer.

120. The method of claim 115, wherein the second antigen binding site of the protein binds TNC, and wherein the cancer to be treated is selected from the group consisting of cervical cancer, breast cancer, pancreatic cancer, lung cancer, non-Hodgkin lymphoma, head and neck cancer, colorectal cancer, esophageal cancer, glioma, and prostate cancer.

121. The method of claim 115, wherein the second antigen binding site of the protein binds TNN, and wherein the cancer to be treated is selected from the group consisting of cervical cancer, breast cancer, pancreatic cancer, lung cancer, non-Hodgkin lymphoma, head and neck cancer, colorectal cancer, esophageal cancer, glioma, and prostate cancer.

122. The method of claim 115, wherein the second antigen binding site of the protein binds CSPG4, and wherein the cancer to be treated is selected from the group consisting of melanoma, renal cancer, sarcoma, glioma, head and neck cancer, breast cancer, bladder cancer, lung cancer, and cervical cancer.

123. The method of claim 115, wherein the second antigen binding site of the protein binds BST1, and wherein the cancer to be treated is selected from the group consisting of acute myeloid leukemia, mesothelioma, bladder cancer, and sarcoma.

124. The method of claim 115, wherein the second antigen binding site of the protein binds SELP, and wherein the cancer to be treated is selected from the group consisting of myeloproliferative neoplasms, acute myeloid leukemia, breast cancer, bladder cancer, thyroid cancer, renal cancer, and pancreatic cancer.

125. The method of claim 115, wherein the second antigen binding site of the protein binds CD200, and wherein the cancer to be treated is selected from the group consisting of breast cancer, colorectal cancer, B cell malignancies, multiple myeloma, acute myeloid leukemia, lymphoma, and mesothelioma.

126. The method of claim 115, wherein the second antigen binding site of the protein binds INSR, and wherein the cancer to be treated is selected from the group consisting of prostate cancer, gastric cancer, colorectal cancer, glioblastoma, breast cancer, endometrial cancer, liver cancer, and renal cancer.

127. The method of claim 115, wherein the second antigen binding site of the protein binds ITGA6, and wherein the cancer to be treated is selected from the group consisting of breast cancer, leukemia, prostate cancer, colorectal cancer, renal cancer, head and neck cancer, ovarian cancer, gastric cancer, and lung cancer.

128. The method of claim 115, wherein the second antigen binding site of the protein binds MELTF, and wherein the cancer to be treated is selected from the group consisting of breast cancer, lung cancer, melanoma, bladder cancer, renal cancer, sarcoma, head and neck cancer, mesothelioma, pancreatic cancer.

129. The method of claim 115, wherein the second antigen binding site of the protein binds PECAM1, and wherein the cancer to be treated is a solid tumor.

130. The method of claim 129, wherein the solid tumor has significant neovasculature.

131. The method of claim 115, wherein the second antigen binding site of the protein binds SLC1A5, and wherein the cancer to be treated is selected from the group consisting of lung cancer, colorectal cancer, breast cancer, prostate cancer, renal cancer, head and neck cancer, neuroblastoma, gastric cancer, and ovarian cancer.
